SEQUENCE LISTING (90 sequences)

SEQ ID NO: 1 - DNA, 24 bp, synthetic construct
  source 1..24: mol_type = other DNA
  misc_feature 1..24: note = human COL1A1 Forward primer
acgaagacatcccaccaatcacct

SEQ ID NO: 2 - DNA, 24 bp, synthetic construct
  source 1..24: mol_type = other DNA
  misc_feature 1..24: note = human COL1A1 Reverse primer
agatcacgtcatcgcacaacacct

SEQ ID NO: 3 - DNA, 24 bp, synthetic construct
  source 1..24: mol_type = other DNA
  misc_feature 1..24: note = human ATM Forward primer
gcaactgtaccaagaagagtccaa

SEQ ID NO: 4 - DNA, 20 bp, synthetic construct
  source 1..20: mol_type = other DNA
  misc_feature 1..20: note = human ATM Reverse primer
gcaggaaaaagccagatgca

SEQ ID NO: 5 - DNA, 20 bp, synthetic construct
  source 1..20: mol_type = other DNA
  misc_feature 1..20: note = human CTGF Forward primer
ccctcgcggcttaccgactg

SEQ ID NO: 6 - DNA, 20 bp, synthetic construct
  source 1..20: mol_type = other DNA
  misc_feature 1..20: note = human CTGF Reverse primer
ggcgctccactctgtggtct

SEQ ID NO: 7 - DNA, 20 bp, synthetic construct
  source 1..20: mol_type = other DNA
  misc_feature 1..20: note = human NOX4 Forward primer
gctgacgttgcatgtttcag

SEQ ID NO: 8 - DNA, 19 bp, synthetic construct
  source 1..19: mol_type = other DNA
  misc_feature 1..19: note = human NOX4 Reverse primer
cgggagggtgggtatctaa

SEQ ID NO: 9 - DNA, 20 bp, synthetic construct
  source 1..20: mol_type = other DNA
  misc_feature 1..20: note = human MRE11A Forward primer
gtccgtgaggctatgaccag

SEQ ID NO: 10 - DNA, 20 bp, synthetic construct
  source 1..20: mol_type = other DNA
  misc_feature 1..20: note = human MRE11A Reverse primer
gttggttgctgctgagatgc

SEQ ID NO: 11 - DNA, 24 bp, synthetic construct
  source 1..24: mol_type = other DNA
  misc_feature 1..24: note = human GAPDH Forward primer
agcaatgcctcctgcaccaccaac

SEQ ID NO: 12 - DNA, 22 bp, synthetic construct
  source 1..22: mol_type = other DNA
  misc_feature 1..22: note = human GAPDH Reverse primer
ccggaggggccatccacagtct

SEQ ID NO: 13 - DNA, 23 bp, synthetic construct
  source 1..23: mol_type = other DNA
  misc_feature 1..23: note = human ACTA2 Forward primer
gacaatggctctgggctctgtaa

SEQ ID NO: 14 - DNA, 23 bp, synthetic construct
  source 1..23: mol_type = other DNA
  misc_feature 1..23: note = human ACTA2 Reverse primer
atgccatgttctatcgggtactt

SEQ ID NO: 15 - DNA, 20 bp, synthetic construct
  source 1..20: mol_type = other DNA
  misc_feature 1..20: note = human ATR Forward primer
gctatgctactagtgggccg

SEQ ID NO: 16 - DNA, 20 bp, synthetic construct
  source 1..20: mol_type = other DNA
  misc_feature 1..20: note = human ATR Reverse primer
gcacgcggtcacatccttat

SEQ ID NO: 17 - DNA, 21 bp, synthetic construct
  source 1..21: mol_type = other DNA
  misc_feature 1..21: note = human CHEK2 Forward primer
tgacaactactggtttgggag

SEQ ID NO: 18 - DNA, 21 bp, synthetic construct
  source 1..21: mol_type = other DNA
  misc_feature 1..21: note = human CHEK2 Reverse primer
cccacttccctgaaaatccga

SEQ ID NO: 19 - DNA, 18 bp, synthetic construct
  source 1..18: mol_type = other DNA
  misc_feature 1..18: note = human FN1 Forward primer
tgtggttgccttgcacga

SEQ ID NO: 20 - DNA, 21 bp, synthetic construct
  source 1..21: mol_type = other DNA
  misc_feature 1..21: note = human FN1 Reverse primer
gcttgtgggtgtgacctgagt

SEQ ID NO: 21 - DNA, 20 bp, synthetic construct
  source 1..20: mol_type = other DNA
  misc_feature 1..20: note = human PRKDC Forward primer
acctgaagcgccgctataat

SEQ ID NO: 22 - DNA, 21 bp, synthetic construct
  source 1..21: mol_type = other DNA
  misc_feature 1..21: note = human PRKDC Reverse primer
ggaaggaccatctgaatcccc

SEQ ID NO: 23 - DNA, 20 bp, synthetic construct
  source 1..20: mol_type = other DNA
  misc_feature 1..20: note = human F10 Forward primer
gctcggggaaagtctgttca

SEQ ID NO: 24 - DNA, 20 bp, synthetic construct
  source 1..20: mol_type = other DNA
  misc_feature 1..20: note = human F10 Reverse primer
gcaggtctcttccatgcact

SEQ ID NO: 25 - DNA, 20 bp, synthetic construct
  source 1..20: mol_type = other DNA
  misc_feature 1..20: note = human ELN Forward primer
ggccattcctggtggagttc

SEQ ID NO: 26 - DNA, 20 bp, synthetic construct
  source 1..20: mol_type = other DNA
  misc_feature 1..20: note = human ELN Reverse primer
cgagaccagcccctggataa

SEQ ID NO: 27 - DNA, 20 bp, synthetic construct
  source 1..20: mol_type = other DNA
  misc_feature 1..20: note = human COL3A1 Forward primer
aatcaggtagacccggacga

SEQ ID NO: 28 - DNA, 20 bp, synthetic construct
  source 1..20: mol_type = other DNA
  misc_feature 1..20: note = human COL3A1 Reverse primer
ttcgtccatcgaagcctctg

SEQ ID NO: 29 - DNA, 20 bp, synthetic construct
  source 1..20: mol_type = other DNA
  misc_feature 1..20: note = human ASPN Forward primer
acaagagagccaagaagcca

SEQ ID NO: 30 - DNA, 22 bp, synthetic construct
  source 1..22: mol_type = other DNA
  misc_feature 1..22: note = human ASPN Reverse primer
tgggactgaggtcaaacctaaa

SEQ ID NO: 31 - DNA, 25 bp, synthetic construct
  source 1..25: mol_type = other DNA
  misc_feature 1..25: note = human COL11A1 Forward primer
aggaacctcagatagatgagaaaaa

SEQ ID NO: 32 - DNA, 23 bp, synthetic construct
  source 1..23: mol_type = other DNA
  misc_feature 1..23: note = human COL11A1 Reverse primer
tgatttagtagccactgtcctca

SEQ ID NO: 33 - DNA, 21 bp, synthetic construct
  source 1..21: mol_type = other DNA
  misc_feature 1..21: note = human CCL2 Forward primer
agctcgcactctcgcctccag

SEQ ID NO: 34 - DNA, 24 bp, synthetic construct
  source 1..24: mol_type = other DNA
  misc_feature 1..24: note = human CCL2 Reverse primer
ggcattgattgcatctggctgagc

SEQ ID NO: 35 - DNA, 20 bp, synthetic construct
  source 1..20: mol_type = other DNA
  misc_feature 1..20: note = human IL6 Forward primer
ccaggagcccagctatgaac

SEQ ID NO: 36 - DNA, 19 bp, synthetic construct
  source 1..19: mol_type = other DNA
  misc_feature 1..19: note = human IL6 Reverse primer
cccagggagaaggcaactg

SEQ ID NO: 37 - DNA, 20 bp, synthetic construct
  source 1..20: mol_type = other DNA
  misc_feature 1..20: note = human CXCL1 Forward primer
gaaagcttgcctcaatcctg

SEQ ID NO: 38 - DNA, 20 bp, synthetic construct
  source 1..20: mol_type = other DNA
  misc_feature 1..20: note = human CXCL1 Reverse primer
caccagtgagcttcctcctc

SEQ ID NO: 39 - DNA, 18 bp, synthetic construct
  source 1..18: mol_type = other DNA
  misc_feature 1..18: note = human LIF Forward primer
tcttggcggcaggagttg

SEQ ID NO: 40 - DNA, 20 bp, synthetic construct
  source 1..20: mol_type = other DNA
  misc_feature 1..20: note = human LIF Reverse primer
gttgttgtgacatgggtggc

SEQ ID NO: 41 - DNA, 22 bp, synthetic construct
  source 1..22: mol_type = other DNA
  misc_feature 1..22: note = mouse ATM Forward primer
cgatatgccagtcttttcaggg

SEQ ID NO: 42 - DNA, 20 bp, synthetic construct
  source 1..20: mol_type = other DNA
  misc_feature 1..20: note = mouse ATM Reverse primer
ctcaaggctgcccttactca

SEQ ID NO: 43 - DNA, 20 bp, synthetic construct
  source 1..20: mol_type = other DNA
  misc_feature 1..20: note = mouse HPRT Forward primer
gttgggcttacctcactgct

SEQ ID NO: 44 - DNA, 20 bp, synthetic construct
  source 1..20: mol_type = other DNA
  misc_feature 1..20: note = mouse HPRT Reverse primer
tcatcgctaatcacgacgct

SEQ ID NO: 45 - DNA, 20 bp, synthetic construct
  source 1..20: mol_type = other DNA
  misc_feature 1..20: note = mouse COL1A1 Forward primer
gtgttccctactcagccgtc

SEQ ID NO: 46 - DNA, 20 bp, synthetic construct
  source 1..20: mol_type = other DNA
  misc_feature 1..20: note = mouse COL1A1 Reverse primer
actcgaacgggaatccatcg

SEQ ID NO: 47 - DNA, 20 bp, synthetic construct
  source 1..20: mol_type = other DNA
  misc_feature 1..20: note = mouse ACTA2 Forward primer
cctcatgccatcatgcgtct

SEQ ID NO: 48 - DNA, 20 bp, synthetic construct
  source 1..20: mol_type = other DNA
  misc_feature 1..20: note = mouse ACTA2 Reverse primer
aatctcacgctcggcagtag

SEQ ID NO: 49 - DNA, 20 bp, synthetic construct
  source 1..20: mol_type = other DNA
  misc_feature 1..20: note = mouse COL3A1 Forward primer
tcctggtggtcctggtactg

SEQ ID NO: 50 - DNA, 20 bp, synthetic construct
  source 1..20: mol_type = other DNA
  misc_feature 1..20: note = mouse COL3A1 Reverse primer
aggagaaccactgttgcctg

SEQ ID NO: 51 - DNA, 20 bp, synthetic construct
  source 1..20: mol_type = other DNA
  misc_feature 1..20: note = mouse COL11A1 Forward primer
cagcgtggtgagactggatt

SEQ ID NO: 52 - DNA, 20 bp, synthetic construct
  source 1..20: mol_type = other DNA
  misc_feature 1..20: note = mouse COL11A1 Reverse primer
ggatggccacgttcacctat

SEQ ID NO: 53 - DNA, 20 bp, synthetic construct
  source 1..20: mol_type = other DNA
  misc_feature 1..20: note = mouse COL1A2 Forward primer
gctggtgtaatgggtcctcc

SEQ ID NO: 54 - DNA, 20 bp, synthetic construct
  source 1..20: mol_type = other DNA
  misc_feature 1..20: note = mouse COL1A2 Reverse primer
cgaccggcatctccattagg

SEQ ID NO: 55 - DNA, 20 bp, synthetic construct
  source 1..20: mol_type = other DNA
  misc_feature 1..20: note = mouse CTGF Forward primer
agaactgtgtacggagcgtg

SEQ ID NO: 56 - DNA, 20 bp, synthetic construct
  source 1..20: mol_type = other DNA
  misc_feature 1..20: note = mouse CTGF Reverse primer
gtgcaccatctttggcagtg

SEQ ID NO: 57 - DNA, 21 bp, synthetic construct
  source 1..21: mol_type = other DNA
  misc_feature 1..21: note = mouse FN1 Forward primer
gaagacagatgagcttcccca

SEQ ID NO: 58 - DNA, 20 bp, synthetic construct
  source 1..20: mol_type = other DNA
  misc_feature 1..20: note = mouse FN1 Reverse primer
ggttggtgatgaagggggtc

SEQ ID NO: 59 - DNA, 20 bp, synthetic construct
  source 1..20: mol_type = other DNA
  misc_feature 1..20: note = mouse POSTN Forward primer
tcatttagagcagccgccat

SEQ ID NO: 60 - DNA, 20 bp, synthetic construct
  source 1..20: mol_type = other DNA
  misc_feature 1..20: note = mouse POSTN Reverse primer
gcaaagagcgtgaagtgacc

SEQ ID NO: 61 - DNA, 20 bp, synthetic construct
  source 1..20: mol_type = other DNA
  misc_feature 1..20: note = mouse VCAN Forward primer
ctggctgtggatggtgttgt

SEQ ID NO: 62 - DNA, 20 bp, synthetic construct
  source 1..20: mol_type = other DNA
  misc_feature 1..20: note = mouse VCAN Reverse primer
cagagtgtacctgctggtgg

SEQ ID NO: 63 - DNA, 21 bp, synthetic construct
  source 1..21: mol_type = other DNA
  misc_feature 1..21: note = mouse CTHRC1 Forward primer
tgaagcaaaaagcgctgatcc

SEQ ID NO: 64 - DNA, 20 bp, synthetic construct
  source 1..20: mol_type = other DNA
  misc_feature 1..20: note = mouse CTHRC1 Reverse primer
actcctgctggtccttgtag

SEQ ID NO: 65 - DNA, 22 bp, synthetic construct
  source 1..22: mol_type = other DNA
  misc_feature 1..22: note = mouse ELN Forward primer
agtctattatccaggggctggt

SEQ ID NO: 66 - DNA, 20 bp, synthetic construct
  source 1..20: mol_type = other DNA
  misc_feature 1..20: note = mouse ELN Reverse primer
ctgctccaaacgttcccaga

SEQ ID NO: 67 - DNA, 20 bp, synthetic construct
  source 1..20: mol_type = other DNA
  misc_feature 1..20: note = mouse ASPN Forward primer
gggatagaaccaggggcatt

SEQ ID NO: 68 - DNA, 21 bp, synthetic construct
  source 1..21: mol_type = other DNA
  misc_feature 1..21: note = mouse ASPN Reverse primer
gttggtggtaggccttttgga

SEQ ID NO: 69 - DNA, 21 bp, synthetic construct
  source 1..21: mol_type = other DNA
  misc_feature 1..21: note = mouse CXCL10 Forward primer
ccacgtgttgagatcattgcc

SEQ ID NO: 70 - DNA, 20 bp, synthetic construct
  source 1..20: mol_type = other DNA
  misc_feature 1..20: note = mouse CXCL10 Reverse primer
tcactccagttaaggagccc

SEQ ID NO: 71 - DNA, 20 bp, synthetic construct
  source 1..20: mol_type = other DNA
  misc_feature 1..20: note = mouse IRF3 Forward primer
aaccctgcaccccaagaaaa

SEQ ID NO: 72 - DNA, 21 bp, synthetic construct
  source 1..21: mol_type = other DNA
  misc_feature 1..21: note = mouse IRF3 Forward primer
acctcgaactcccattgttcc

SEQ ID NO: 73 - DNA, 19 bp, synthetic construct
  source 1..19: mol_type = other DNA
  misc_feature 1..19: note = mouse ISG15 Forward primer
ctagagctagagcctgcag

SEQ ID NO: 74 - DNA, 19 bp, synthetic construct
  source 1..19: mol_type = other DNA
  misc_feature 1..19: note = mouse ISG15 Reverse primer
agttagtcacggacaccag

SEQ ID NO: 75 - DNA, 20 bp, synthetic construct
  source 1..20: mol_type = other DNA
  misc_feature 1..20: note = mouse IFIT1 Forward primer
caaggcaggtttctgaggag

SEQ ID NO: 76 - DNA, 20 bp, synthetic construct
  source 1..20: mol_type = other DNA
  misc_feature 1..20: note = mouse IFIT1 Reverse primer
gacctggtcaccatcagcat

SEQ ID NO: 77 - DNA, 20 bp, synthetic construct
  source 1..20: mol_type = other DNA
  misc_feature 1..20: note = mouse CCL2 Forward primer
cactcacctgctgctactca

SEQ ID NO: 78 - DNA, 24 bp, synthetic construct
  source 1..24: mol_type = other DNA
  misc_feature 1..24: note = mouse CCL2 Reverse primer
gagcttggtgacaaaaactacagc

SEQ ID NO: 79 - DNA, 21 bp, synthetic construct
  source 1..21: mol_type = other DNA
  misc_feature 1..21: note = mouse CCL5 Forward primer
atcttgcagtcgtgtttgtca

SEQ ID NO: 80 - DNA, 21 bp, synthetic construct
  source 1..21: mol_type = other DNA
  misc_feature 1..21: note = mouse CCL5 Reverse primer
acccacttcttctctgggttg

SEQ ID NO: 81 - DNA, 24 bp, synthetic construct
  source 1..24: mol_type = other DNA
  misc_feature 1..24: note = mouse IL6 Forward primer
tgattgtatgaacaacgatgatgc

SEQ ID NO: 82 - DNA, 24 bp, synthetic construct
  source 1..24: mol_type = other DNA
  misc_feature 1..24: note = mouse IL6 Reverse primer
ggactctggctttgtctttcttgt

SEQ ID NO: 83 - DNA, 20 bp, synthetic construct
  source 1..20: mol_type = other DNA
  misc_feature 1..20: note = mouse LIF Forward primer
agcgccaatgctctcttcat

SEQ ID NO: 84 - DNA, 20 bp, synthetic construct
  source 1..20: mol_type = other DNA
  misc_feature 1..20: note = mouse LIF Reverse primer
cttctctgtcccgttgccat

SEQ ID NO: 85 - DNA, 20 bp, synthetic construct
  source 1..20: mol_type = other DNA
  misc_feature 1..20: note = mouse CXCL1 Forward primer
actcaagaatggtcgcgagg

SEQ ID NO: 86 - DNA, 21 bp, synthetic construct
  source 1..21: mol_type = other DNA
  misc_feature 1..21: note = mouse CXCL1 Reverse primer
acttggggacaccttttagca

SEQ ID NO: 87 - DNA, 21 bp, synthetic construct
  source 1..21: mol_type = other DNA
  misc_feature 1..21: note = mouse CXCL12 Forward primer
tgcatcagtgacggtaaacca

SEQ ID NO: 88 - DNA, 20 bp, synthetic construct
  source 1..20: mol_type = other DNA
  misc_feature 1..20: note = mouse CXCL12 Reverse primer
gcgatgtggctctcgaagaa

SEQ ID NO: 89 - DNA, 21 bp, synthetic construct
  source 1..21: mol_type = other DNA
  misc_feature 1..21: note = mouse CXCL2 Forward primer
gcccagacagaagtcatagcc

SEQ ID NO: 90 - DNA, 19 bp, synthetic construct
  source 1..19: mol_type = other DNA
  misc_feature 1..19: note = mouse CXCL2 Reverse primer
ggcttcagggtcaaggcaa

(19) United States
(12) Patent Application Publication
(10) Pub. No.: US 2026/0256799 A1
(43) Pub. Date: Sep. 3, 2026

(54) INHIBITORS FOR TREATING SOLID TUMOURS

(71) Applicant: UNIVERSITY OF SOUTHAMPTON, Southampton (GB)

(72) Inventors: Gareth THOMAS, Southampton (GB); Massimiliano MELLONE, Southampton (GB)

(21) Appl. No.: 19/162,588

(22) PCT Filed: Mar. 10, 2023
(86) PCT No.: PCT/GB2023/050572

§ 371 Date: Sep. 5, 2025

(51) Int. Cl. A61K 31/5377; A61K 31/404; A61K 31/4188; A61K 31/425; A61K 31/437; A61K 31/4535; A61K 31/497; A61K 31/517; A61K 45/06; A61P 35/00

(52) U.S. Cl. CPC A61K 31/5377; A61K 31/404; A61K 31/4188; A61K 31/425; A61K 31/437; A61K 31/4535; A61K 31/497; A61K 31/517; A61K 45/06; A61P 35/00

(57) ABSTRACT

The present invention provides a new method of treating a solid tumour with an ATM (ataxia telangiectasia mutated) inhibitor and/or a CHK2 (checkpoint kinase 2) inhibitor. New therapy combinations, formulations and kits are also provided.

The present invention provides a new method of treating a solid tumour with an ATM (ataxia telangiectasia mutated) inhibitor and/or a CHK2 (checkpoint kinase 2) inhibitor. New therapy combinations, formulations and kits are also provided.

### BACKGROUND

Immune-checkpoint blockade elicits durable anti-tumour clinical responses in an increasing number of malignancies. However, its success is limited to a fraction of cancer patients, highlighting the need to identify targetable resistance mechanisms to widen its clinical effectiveness [1]. Analysis of human cancers and mouse models has shown that non-responsiveness to immune-checkpoint blockade can result from limited T cell infiltration, mediated by the cells of the tumour microenvironment [1].

Among the heterogeneous cancer-associated fibroblast (CAF) population, a distinct myofibroblastic phenotype has been found in most types of solid tumour [2]. Myofibroblastic CAF (myoCAF) are analogous to myofibroblasts found in wound healing and tissue fibrosis. These contractile, secretory cells are regulated through TGF-β1-signaling, express α-smooth muscle actin (SMA) and secrete collagen-rich extracellular matrix (ECM) [2, 3]. Studies in multiple cancer types have shown that tumours with an SMA-positive, myoCAF-high stroma are associated with poor prognosis [4-6] and that myoCAF contribute to many ‘hallmarks of malignancy [7]. Transcriptomic analyzes of tumours that fail to respond to anti-PD-1/PD-L1 have identified upregulation of myoCAF genes [8-13], suggesting that myoCAF mediate resistance to checkpoint blockade. Consistent with this, myoCAF-high cancers contain low levels of infiltrating T-cells [8, 10, 14] and functional studies in murine models have confirmed that myoCAF promote resistance to different types of immunotherapy [8, 13, 14], in part by promoting T-cell exclusion from tumours. This has led the emergence of myoCAF as potential therapeutic targets [15].

TGF-β/SMAD signaling is recognized as the central pathway regulating myofibroblast/myoCAF differentiation, although other pathways also influence the myoCAF phenotype [3]. Previously, the inventors found that during TGF-β1-induced differentiation, myofibroblasts upregulate a number of genes associated with DNA repair, suggesting that the DNA damage response (DDR) pathway may be triggered during the process [20]. The DDR is a complex cellular network of coordinated pathways that maintain genome integrity by detecting and repairing DNA lesions [21], orchestrated by DNA damage-sensing kinases Ataxia-telangiectasia mutated (ATM), Ataxia-telangiectasia mutated and Rad3-related (ATR), or DNA-dependent protein kinase (DNA-PKcs) [22]. TGF-β/SMAD signaling has been reported to contribute to the cellular DDR induced by genotoxic insults [23, 24] or by radiation/stress-driven bystander effects [25-27].

However, limited understanding of CAF heterogeneity and of the mechanisms regulating the accumulation of different CAF phenotypes has resulted in unsuccessful clinical trials, illustrating challenges to effectively target these cells.

Improved methods and compositions for treating solid tumours are needed.

### BRIEF SUMMARY OF THE DISCLOSURE

The inventors sought to investigate the role of the DDR signaling in regulating myoCAF phenotype and function using in vitro/ex vivo culture of myofibroblasts/myoCAF, analysis of human tumours, and CAF-rich murine tumour models that recapitulate the stromal morphology, immune microenvironment and immunotherapy resistance found in CAF-rich human tumours. The inventors surprisingly found that ATM and/or CHK2 inhibition reversed myoCAF differentiation and potentiated immunotherapy response in myoCAF-high or myoCAF-moderate tumours without affecting control (CAF-low) tumours comprising TC-1 and MC38 cells alone.

Accordingly, in one aspect the invention relates to a method of treating a solid tumour in a subject, the method comprising administering a therapeutically effective amount of an ATM inhibitor and/or a CHK2 inhibitor to the subject, wherein the solid tumour is a myofibroblastic cancer-associated fibroblast (myoCAF)-high or myoCAF-moderate tumour (also referred to herein as tumours with high or moderate levels of myoCAF respectively).

In another aspect the invention relates to a method of treating a solid tumour in a subject, the method comprising administering a therapeutically effective amount of:
a) an ATM inhibitor and/or a CHK2 inhibitor; and
   b) at least one immunotherapeutic agent;
   to the subject, wherein the inhibitor and the at least one immunotherapeutic agent are administered simultaneously, separately or consecutively, and wherein the solid tumour is a myoCAF-high or -moderate tumour.

In another aspect the invention relates to a method of treating a solid tumour in a subject, the method comprising administering a therapeutically effective amount of an ATM inhibitor and/or a CHK2 inhibitor to the subject, wherein the subject is undergoing treatment with, has been treated with, or has been prescribed treatment with at least one immunotherapeutic agent, and wherein the solid tumour is a myoCAF-high or -moderate tumour.

In another aspect the invention relates to a method of treating a solid tumour in a subject, the method comprising administering a therapeutically effective amount of at least one immunotherapeutic agent to the subject, wherein the subject is undergoing treatment with, has been treated with, or has been prescribed treatment with an ATM inhibitor and/or a CHK2 inhibitor, and wherein the solid tumour is a myoCAF-high or -moderate tumour.

In another aspect the invention relates to an ATM inhibitor and/or a CHK2 inhibitor for use in treating a solid tumour, wherein the solid tumour is a myoCAF-high or -moderate tumour.

In another aspect the invention relates to an ATM inhibitor and/or a CHK2 inhibitor for use in treating a solid tumour in combination with at least one immunotherapeutic agent, wherein the solid tumour is a myoCAF-high or -moderate tumour.

In another aspect the invention relates to an immunotherapeutic agent for use in treating a solid tumour in combination with an ATM inhibitor and/or a CHK2 inhibitor, wherein the solid tumour is a myoCAF-high or -moderate tumour.

In another aspect the invention relates to an ATM inhibitor and/or a CHK2 inhibitor in combination with at least one immunotherapeutic agent for use in treating a solid tumour, wherein the solid tumour is a myoCAF-high or -moderate tumour.

In another aspect the invention relates to a pharmaceutical formulation comprising an ATM inhibitor and/or a CHK2 inhibitor, together with at least one immunotherapeutic agent, and at least one pharmaceutically acceptable excipient.

In another aspect the invention relates to a kit comprising two separate formulations to be used together, the formulations being:
a) a first formulation comprising an ATM inhibitor and/or a CHK2 inhibitor and at least one pharmaceutically acceptable excipient; and
b) a second formulation comprising at least one immunotherapeutic agent and at least one pharmaceutically acceptable excipient.

Suitably, the pharmaceutical formulation and the kit may be used in treating a solid tumour, wherein the solid tumour is a myoCAF-high or -moderate tumour.

In another aspect the invention relates to a method for promoting T-cell infiltration and antitumour immunity of a solid tumour in a subject, comprising subjecting the subject to a therapeutically effective amount of an ATM inhibitor and/or a CHK2 inhibitor in combination with at least one immunotherapeutic agent, wherein the solid tumour is a myoCAF-high or -moderate tumour.

Suitably, the subject may be identified as having a solid tumour a myoCAF-high or -moderate tumour.

Suitably, the ATM inhibitor and/or the CHK2 inhibitor may be used in a neoadjuvant, adjuvant, first line and/or palliative manner.

Suitably, the solid tumour may be:
(a) a myoCAF-high tumour when α-smooth muscle actin (SMA) staining of a tumour tissue sample shows a stromal positivity of more than 50%; or
(b) a myoCAF-moderate tumour when α-smooth muscle actin (SMA) staining of a tumour tissue sample shows a stromal positivity of 5-50%.

Suitably, the solid tumour may be a cancer selected from the group consisting of: non-small cell lung cancer (NSCLC), head and neck squamous cell cancer (HNSCC), esophageal cancer, ovarian cancer, colorectal cancer, liver cancer, breast cancer, ovarian cancer, cervical cancer, endometrial cancer, melanoma, hepatocellular carcinoma, kidney cancer, prostate cancer, gastric cancer, pancreatic cancer, urinary bladder cancer, and brain cancer; optionally wherein the brain cancer is glioblastoma.

Suitably, the ATM and/or the CHK2 inhibitor may prevent or reverse the differentiation of myoCAFs.

Suitably, the ATM inhibitor may directly inhibit ATM and/or wherein the CHK2 inhibitor may directly inhibit CHK2.

Suitably, the ATM inhibitor may inhibit ATM by blocking the kinase function of ATM and/or wherein the CHK2 inhibitor may inhibit CHK2 by blocking the kinase function of CHK2.

Suitably, the ATM and/or the CHK2 inhibitor may promote intra-tumoural T-cell infiltration into the solid tumour.

Suitably, the ATM and/or the CHK2 inhibitor may potentiate the solid tumour's response to anti-PD-1 blockade.

Suitably, the ATM inhibitor may be selected from the group consisting of KU-55933, KU-60019, KU-59403, CP-466722, AZ31/AZ32, AZD0156, and AZD1390.

Suitably, the CHK2 inhibitor may be selected from the group consisting of CCT241533, BML-277, PHI-101, VRX0466617, PV1019, AZD7762, Y2606368 (Prexasertib).

Suitably, the immunotherapeutic agent may be selected from the group consisting of: immune checkpoint inhibitor, a cancer vaccine and/or an immune cell.

Suitably, the immunotherapeutic agent may be an immune checkpoint inhibitor, optionally wherein the immune checkpoint inhibitor is selected from the group consisting of a: PD-1 inhibitor, PD-L1 inhibitor, CTLA4 inhibitor, TIGIT inhibitor, TIM-3 inhibitor, LAG-3 inhibitor, B7-H3 inhibitor, B7-H4 inhibitor.

In another aspect the invention refers to a method of determining whether a subject having a solid tumour is likely to benefit from treatment with an ATM and/or a CHK2 inhibitor, the method comprising carrying out α-smooth muscle actin (SMA) staining of a tumour tissue sample and identifying that the subject is likely to benefit from treatment with an ATM and/or the CHK2 inhibitor if the staining shows a stromal positivity of at least 5%.

In another aspect the invention refers to a method of determining a suitable treatment regimen for a subject having a solid tumour, the method comprising: carrying out α-smooth muscle actin (SMA) staining of a tumour tissue sample, wherein a stromal positivity of at least 5% in the staining indicates that a suitable treatment regimen will comprise treatment of the subject with an ATM and/or a CHK2 inhibitor.

Various aspects of the invention are described in further detail below.

### BRIEF DESCRIPTION OF THE FIGURES

Embodiments of the invention are further described hereinafter with reference to the accompanying drawings, in which:

FIG. 1: ATM activation in myofibroblasts.

a&b) Western blotting of HFFF2 treated with TGF-β1 over time. c,d) Representative immunofluorescence staining of pATM- (c), pH2AX-positive (d) foci and quantification of HFFF2 treated over time with TGF-β1 or NCS (positive control); nuclei are outlined by dotted white lines based on Dapi nuclear counter-staining (not shown; scale bar=10 μm). Foci counts are expressed as % of total cell number per FoV (FoV=nₜᵣ≥8); SD and Kruskall-Wallis test are shown. e) Alkaline comet assay of HFFF2 treated with TGF-β1 for 24 hours or irradiated with 2Gy (as positive control; nₜᵣ=50; homoscedastic Student's t-test refers to the control). f) Analysis of viability, proliferation and cell death monitored by MTT, thymidine incorporation and propidium iodide (PI) staining respectively. HFFF2 were treated for 3 days with TGF-β1 or with H₂O₂ or cisplatin as positive controls (nₜᵣ=2-3). g) Senescence assay of HFFF2 treated with TGF-β1 over time. The % of SA-β-Gal-positive cells is expressed as fold induction compared with untreated control; FoV=nₜᵣ=10. h) Western blotting of NSCLC and HNSCC CAF. i) Volcano plots with FDRQ (significance) and NES (correlation) of GSEA performed on the indicated datasets from LCMD tumour vs normal stroma. The dotted line drawn at 1.3 of −log 10 FDRQ axis indicates FDRQ=0.05. j-n) Representative image of HNSCC MxIHC; bright field image of cytokeratin staining (scale bar=5 mm j); pseudo-colored images (scale bar=100 μm k; scale bar=20 μm I&m); single-stained and merged pseudo-colored images with examples of the cell regions used for the quantification highlighted for pATM or SMA positivity (subtracted CD31 staining labelled also); Quantification of pATM positivity in SMA+ cells (negative for CD31 & CK) in 10 HNSCC and 10 NSCLC cases (n) with significance calculated comparing pATM+/SMA+vs pATM−/SMA+ CAFs. Paired Student's t-test is used in the figure and refers to control unless otherwise stated.

FIG. 2: ATM inhibition suppresses myofibroblast differentiation. a,b) Western blotting and its quantification of HFFF2 treated for 72 hours with TGF-β1±ATM inhibitors [13.3 μM KU55933 (a); 2.5 μM KU60019 (b)]. c) Western blotting quantification of SMA expression in primary fibroblasts isolated ex vivo from colon (n=2), skin (n=1) and oral (n=2) tissues from healthy donors. Fibroblasts were treated as in a (see also FIG. 8c-g). d) Collagen gel contraction assay and measurement of gel area. HFFF2 or normal primary oral fibroblasts were treated as in a. Representative gel images shown on the left (nₜᵣ=2). e) Representative immunofluorescence staining of SMA-positive stress fibers or collagen1 deposition in HFFF2 treated with TGF-β1+/−ATM inhibitor as above for 3 (SMA) or 7 (Collagen1) days; relative quantification of the mean (FoV for both SMA and Collagen1=10); scale bars for SMA=100 μm and for collagen1=500 μm). f,g) Western blotting/quantification (f) and Q-RT-PCR (nₜᵣ=3) (g) of HFFF2 transfected as indicated and treated with TGF-β1 for 72 hours (ACTA2=SMA gene). h,i) Western blotting and quantification of HFFF2 fibroblast treated for 72 hours with TGF-β1+/−ATR inhibitor (VE891; 0.5 μM & 2.5 μM; h) or +/−DNAPKcs inhibitor (2 μM & 10 μM; i) for 72 hours. j,k) Q-RT-PCR of HFFF2 transfected as indicated and treated with TGF-β1 for 72 hours (nₜᵣ=3; PRKDC=DNAPKcs). 1) Western blotting and quantification of HFFF2 treated for 72 hours with TGF-β1±CHK2 inhibitor (1.5 μM CCT241533). m) Representative immunofluorescence staining of SMA and collagen1 in HFFF2 treated with TGF-β1±CHK2 inhibitor (quantified as in e). n,o) Western blotting/quantification (n) and Q-RT-PCR (o; nₜᵣ=3) of HFFF2 transfected as indicated and treated with TGF-β1 for 72 hours. Heteroscedastic Student's t-test is used throughout the figure and is relative to TGF-β1-treated samples unless otherwise highlighted.

FIG. 3: ATM inhibition reverses the myofibroblast CAF phenotype and inhibits function.

a-c) Western blotting of HNSCC (a) and NSCLC (b) CAF treated with KU55933 for 7 days and their quantification (c). d) Q-RT-PCR of CAF (NSCLC, n=1); HNSCC, n=2) stably expressing either shCTR or shATM (nₜᵣ=3). e) Collagen gel contraction assay and measurement of gel area of shCTR or shATM HNSCC and NSCLC CAF (representative gel images are shown on the left; nₜᵣ=2). f,g) Transwell invasion assays of H441 (f) or 5PT (g) cells towards CAF-conditioned media generated from NSCLC (n_{br}=3, nₜᵣ=3-5; f) and HNSCC (n_{br}=8; nₜᵣ=2-4; g) treated as in a (means are shown and Two Way Anova is used and refers to the control).

Heteroscedastic Student T-test is shown throughout the figure and refers to the control unless otherwise stated.

FIG. 4: TGFβ activates ATM via NOX4-driven DNA damage/MRN complex and oxidation.

a-e HFFF2 were treated with TGF-β1 for 24 hours. a) Western blotting/quantification of HFFF2 treated with TGFβR1 inhibitor; b) Western blotting/quantification of nucleus/cytoplasm extracts using HSP90 and 53BP1 as cytoplasmic and nuclear marker respectively. c) Representative immunofluorescence staining for NOX4 and pATM (scale bar=5 μm). d) Western blotting/quantification of HFFF2 transfected as indicated. e) Western blotting/quantification of HFFF2 treated with inhibitors of MRE complex inhibitor (Mirin, 40 μM) or NOX4 (GKT137831; 40 μM). f,g) Q-RT-PCR (f; nₜᵣ=3) and Western blotting/quantification (g) of HFFF2 treated TGF-31 for 72 hours±40 μM Mirin. h,i) Q-RT-PCR (h; nₜᵣ=3) and Western blotting/quantification (i) of IMR90 fibroblasts transfected as indicated and treated with TGF-β1 for 72 hours. j) Representative immunofluorescent staining of SMA-positive stress fibers and relative quantification of the mean in HFFF2 treated with TGF-31 for 72 hours+/−Mirin (40 μM; scale bar=100 μm; FoV=10). k) Western blotting/quantification of HFFF2 transfected as indicated and treated with TGF-β1 for 48 hours; gel run in non-reducing -Dithiothreitol (DTT) conditions for ATM dimer (ATM-D). 1) Western blotting/quantification (±DTT) of NOX4-inducible HEK-293 cells treated with doxycycline over time. m) Western blotting/quantification of NOX4-inducible HEK-293 cells treated with doxycycline±KU55933 for 22 hours. n) Schematic diagram of the main findings in the figure. Heteroscedastic Student's t-test is used in the figure and refers to the TGF-β1 treated samples unless otherwise highlighted.

FIG. 5: Targeting ATM in myofibroblasts reduces their intratumoural accumulation and slows tumour growth.

a,f) Q-RT-PCR showing shRNA ATM knockdown in HFFF2 (a) or TGF-β1-treated MLF (myoMLF) (f) prior to injection in mice (nₜᵣ=3; SD shown). b,g,c,h) Tumour growth curves (b,g) and AUC histograms (c,h) following co-injection of tumour cells with shCTR or shATM fibroblasts (5PT cells+HFFF2, b,c; TC-1 cells+myoMLF, g,h). Data from single experiments are presented; mouse numbers=3-8 (b,g). Two Way Anova is used for AUC analysis of three individual experiments for both 5PT (c) and TC-1 models (h). d,i). Representative SMA IHC from the experiments shown in b and g respectively. e,j) Quantification of SMA staining (nₜᵣ=FoV=3) from the experiments shown in b (e) and g (j). k,l & p,q) Mice injected with either TC-1+/−myoMLF (k,l) or MC38+/−TGF-β1-treated MCF (myoMCF) (p,q) were treated with ATM inhibitor AZD0156 for the duration of the experiment (mouse number=5-8); tumour growth curves (k,p); AUC analysis of two experiments relative to k (two-way Anova, 1); AUC analysis of the single experiment shown in p (homoscedastic Student T-test, q). m,n & r,s) Representative images and quantification of SMA IHC of mouse tumours in k and p respectively (nₜᵣ=FoV=3). o) Overall survival of TC-1+myoMLF mice treated daily with AZD0156 at day 15-28 (mouse number=11-12; Mantel-Cox log-rank test is shown; see also FIG. 11k,l]. Homoscedastic Student T-test is shown in the figure and refers to the control unless otherwise highlighted. All the scale bars=200 μm.

FIG. 6: Targeting myofibroblast ATM promotes tumour CD8 T-cell infiltration and potentiates immunotherapy.

a,b) Representative IHC staining (a) and relative quantification (b) of CD8 T-cells in the core & periphery of TC-1 myo-rich tumours (described in FIG. 5f-j) (nₜᵣ=FoV=10; dotted lines highlight the tumour margins). c,d) Representative IHC staining (c) and relative quantification (d) of CD8 T-cells in the core & periphery of tumours described in FIG. 5k (nₜᵣ=FoV=10). e-f) Flow cytometry analysis from TC-1+myo-rich tumours described in FIG. 5k (e shows two experiments; Two Way Anova is used). g,h) Representative IHC staining (g) and relative quantification (h) of CD8 T-cells in the core & periphery of the tumours described in FIG. 5p (nₜᵣ=FoV=10). i-l) Mice were injected with TC-1+myoMLF and treated with RAH vaccine+/−AZD0156. Control plasmid with vehicle was used as control. Tumour growth curves of a representative experiment (i; mouse number=7-8; see also FIG. 13a); Two-way Anova is shown and refers to AUC analysis of three experiments (j). Representative IHC staining (k) and relative quantification of CD8 T-cells in the tumour core (I; nₜᵣ=FoV=10). m-p) Mice were injected with MC38 and myoMCF and treated with aPD-1 and AZD0156, either alone or in combination. Control mice received isotype control antibody and vehicle. Tumour growth curves of a single experiment (m; mouse number=5-8; see also FIG. 13b) and relative AUC analysis (n). Representative IHC staining (o) and relative quantification (p) of CD8 T-cells in the tumour core (nₜᵣ=FoV=10 in p). q,r) Overall survival of mice injected with TC-1+myoMLF (q) or MC38+myoMCF (r) and treated as indicated (mouse number=7-8 for both experiments; Mantel-Cox log-rank test is shown; see also FIG. 13c&d). Scale bars=200 μm; homoscedastic Student T-test is used throughout the figure and is relative to the control unless otherwise highlighted.

FIG. 7: ATM activation in myofibroblasts.

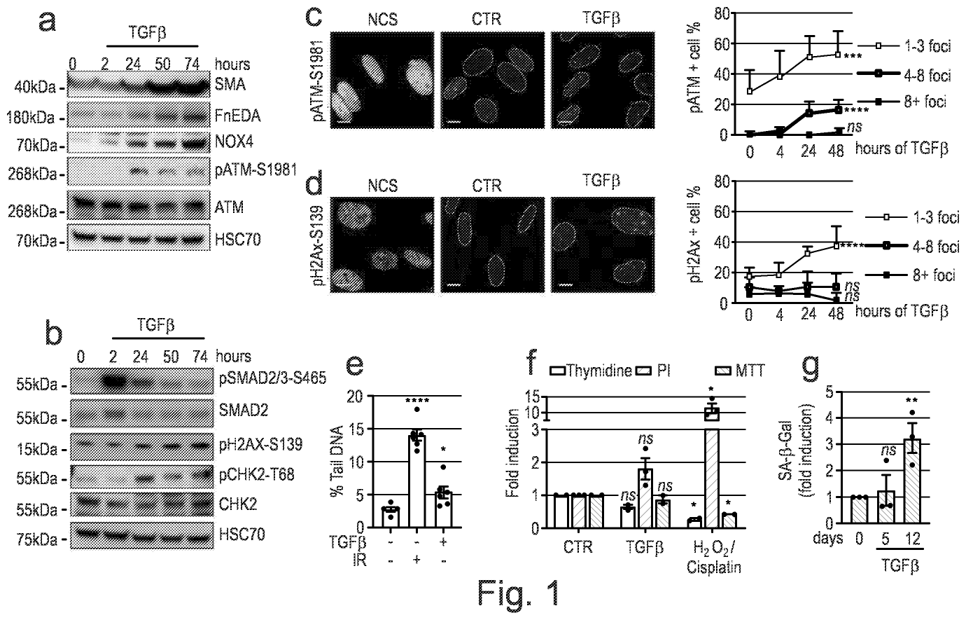

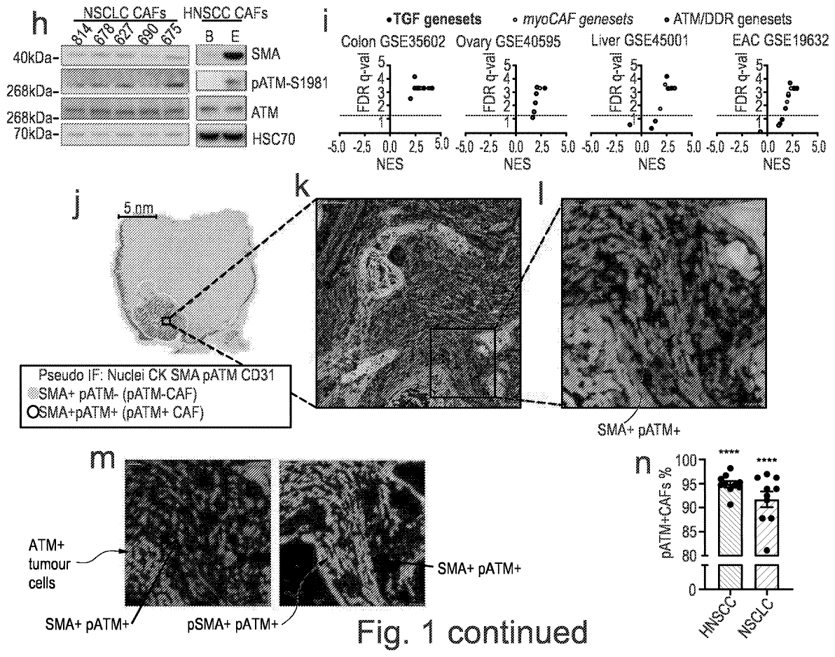

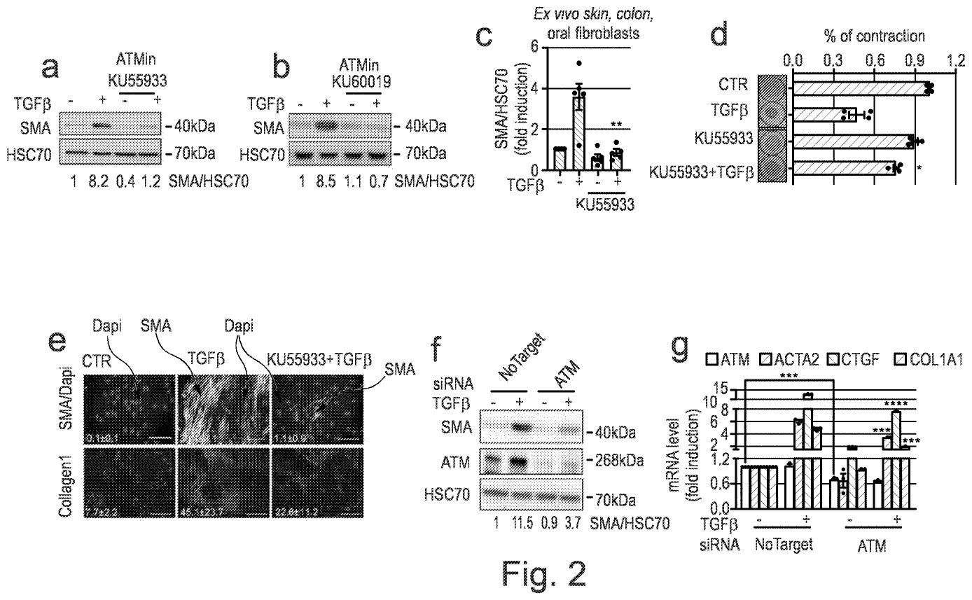

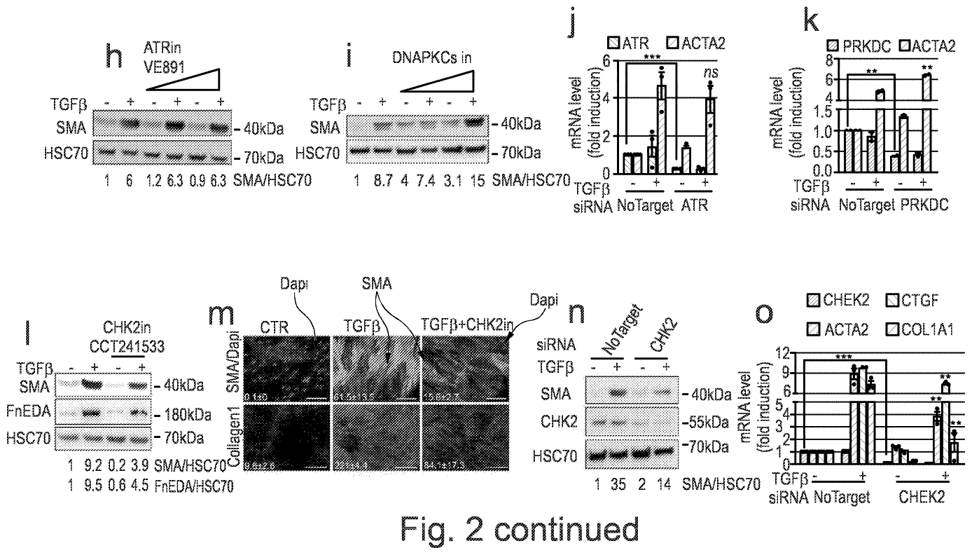

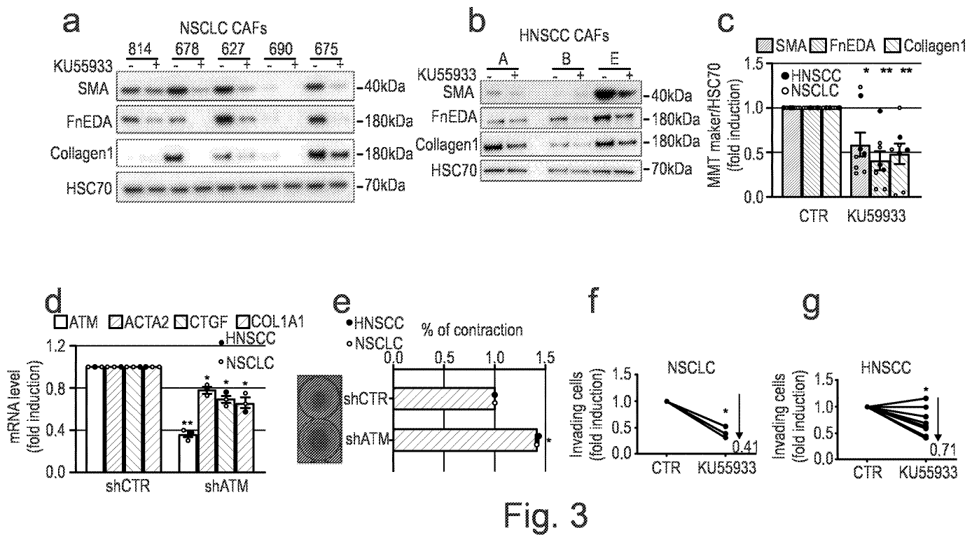

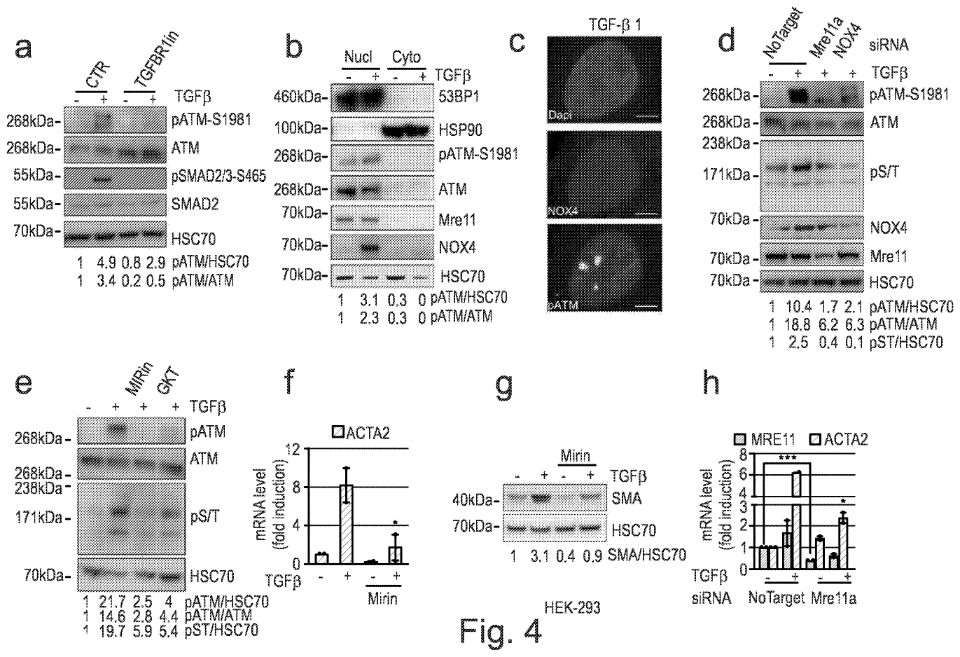

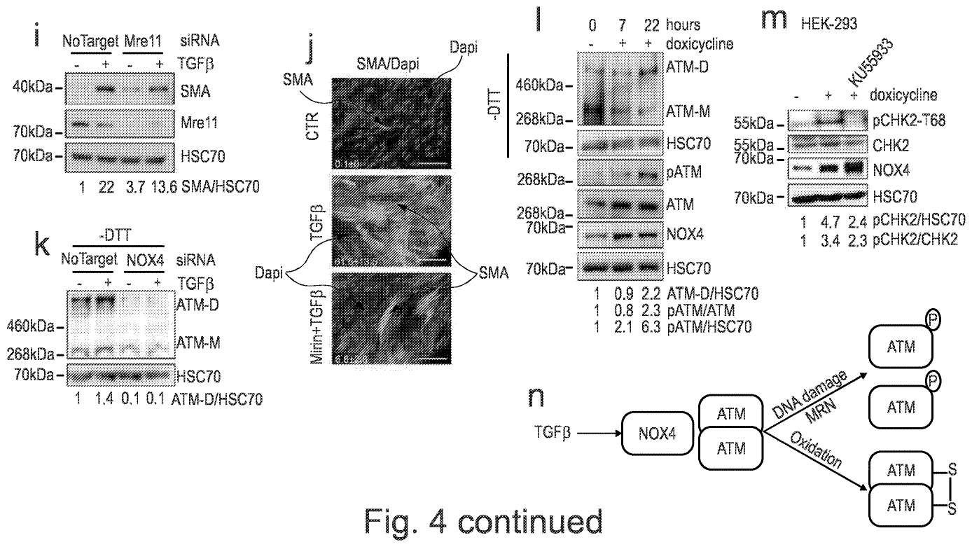

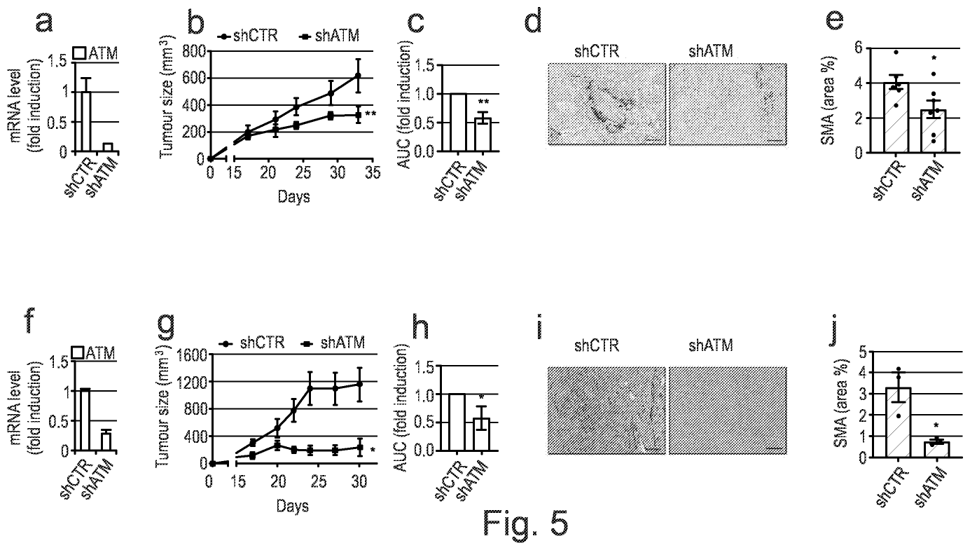

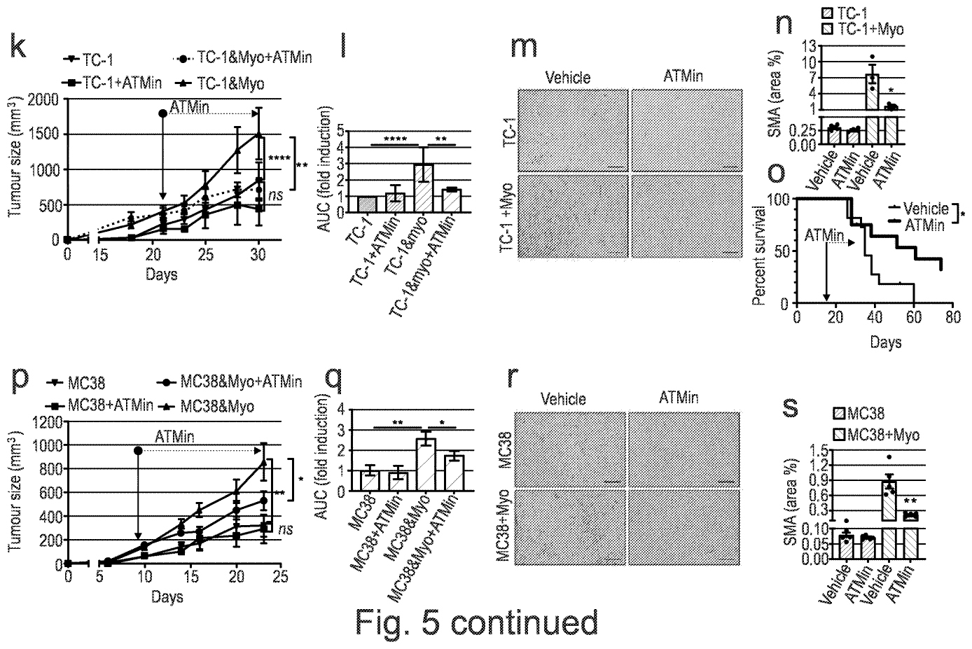

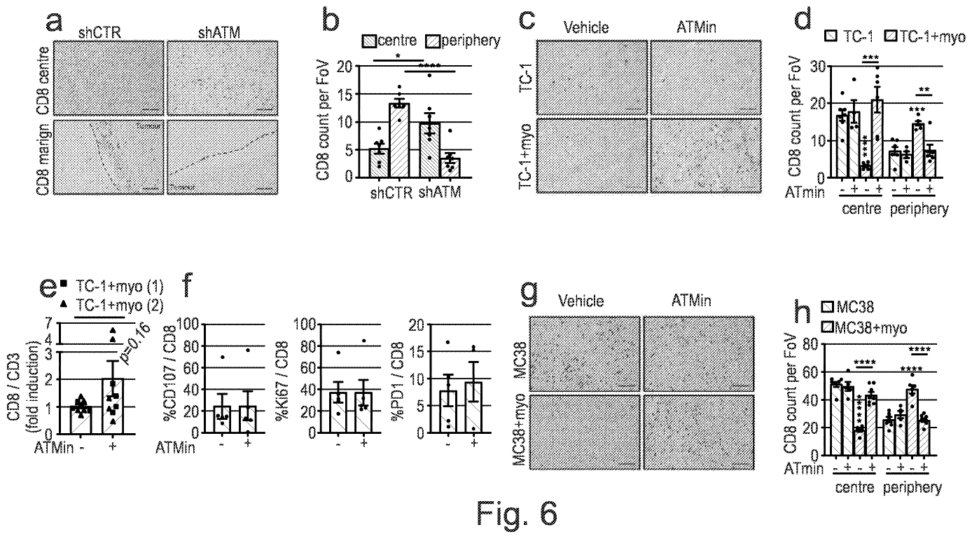

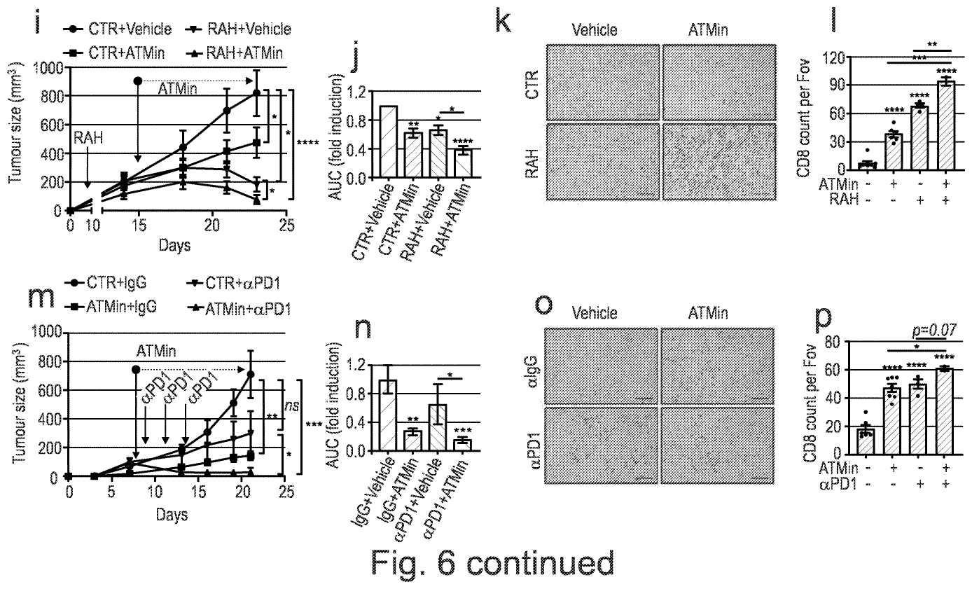

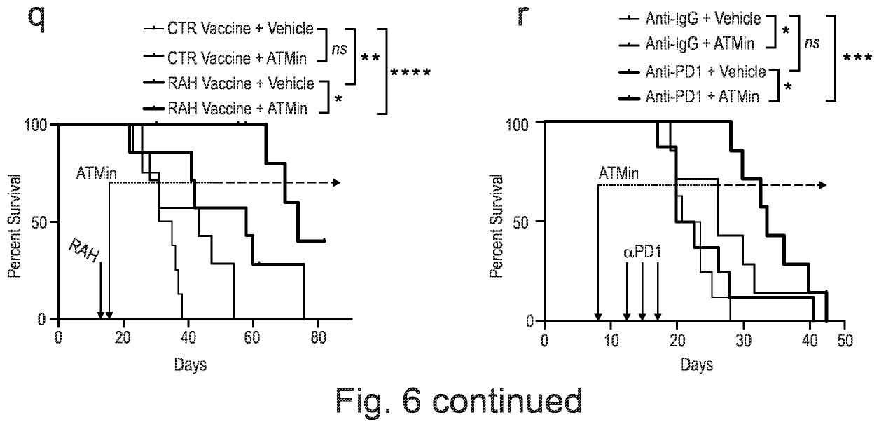

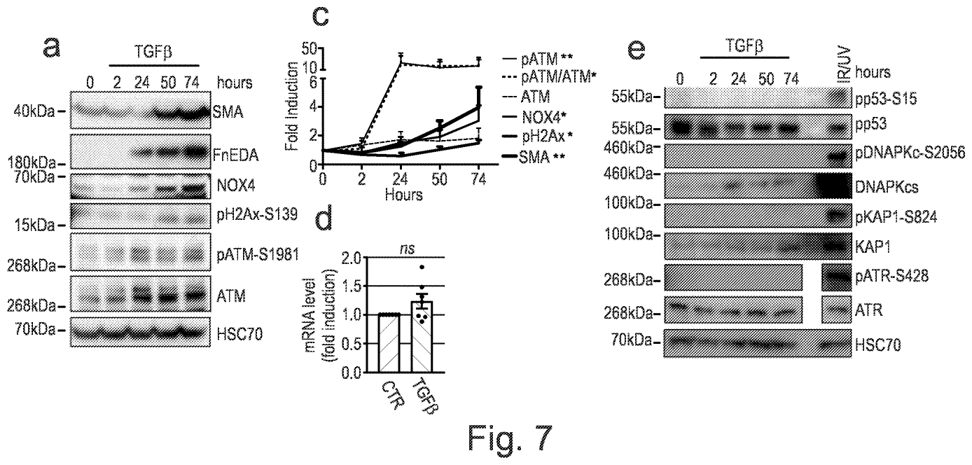

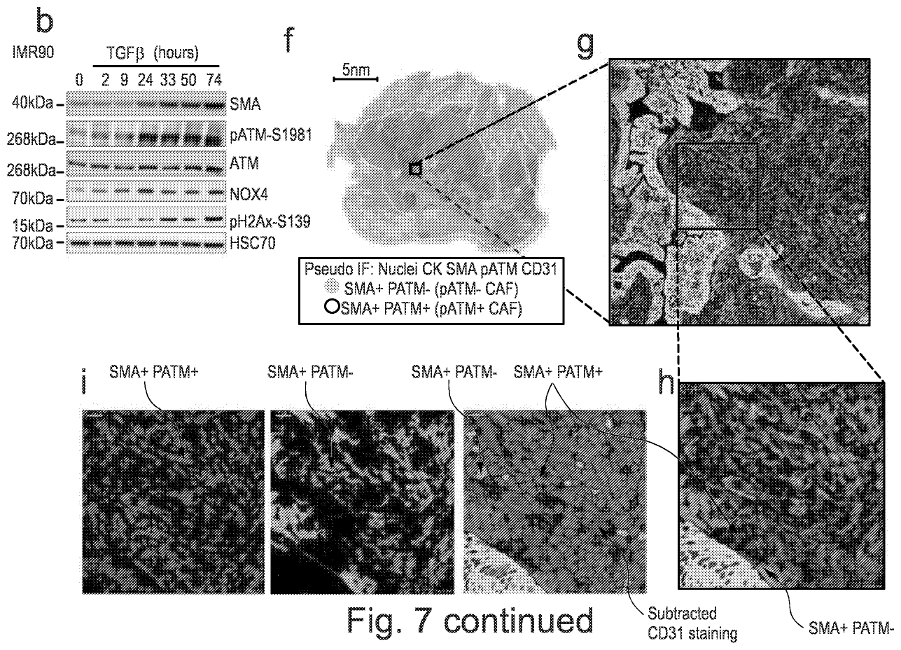

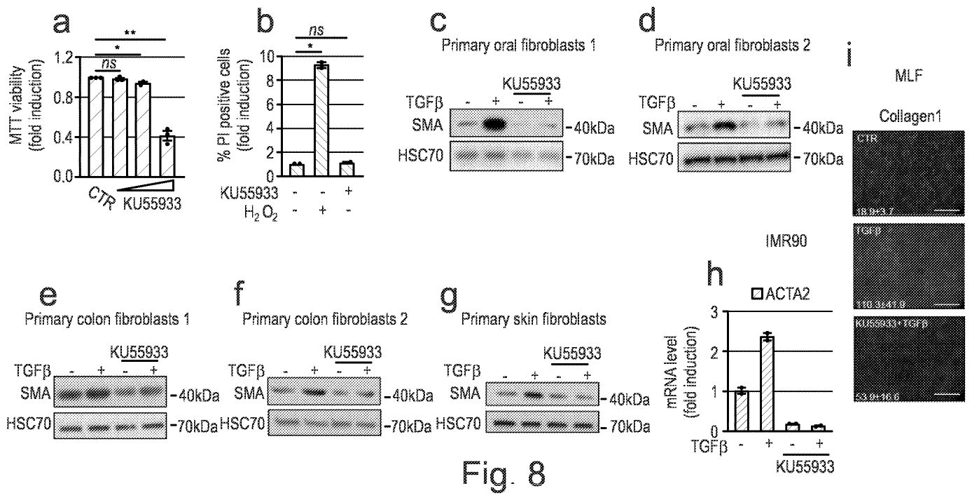

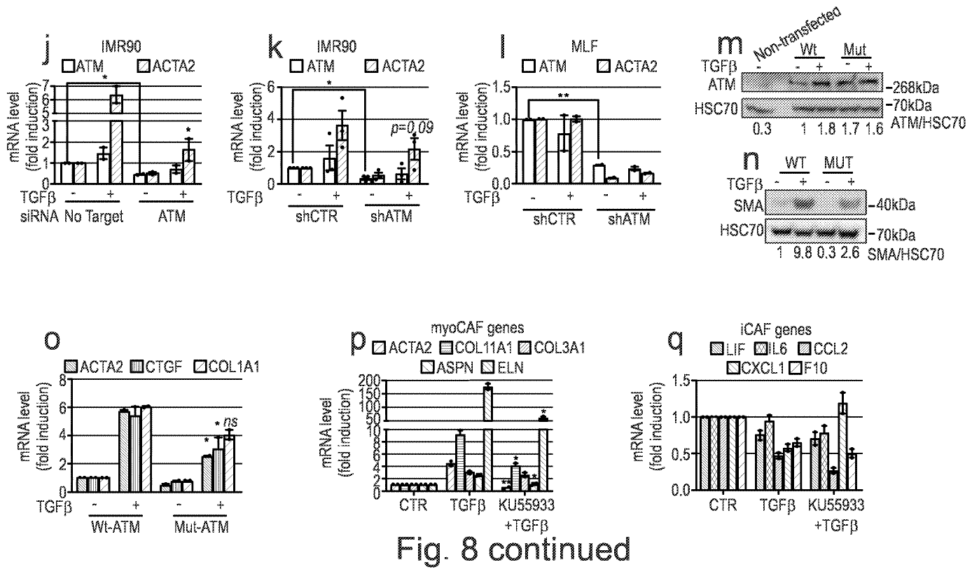

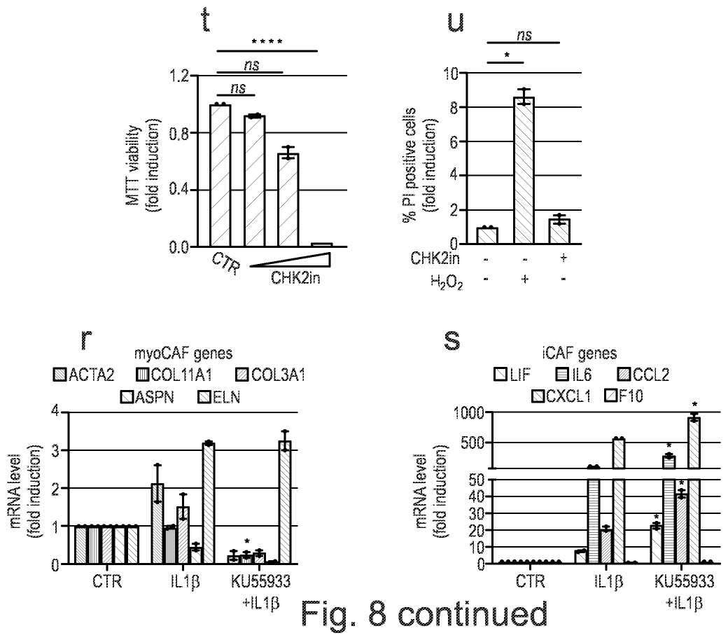

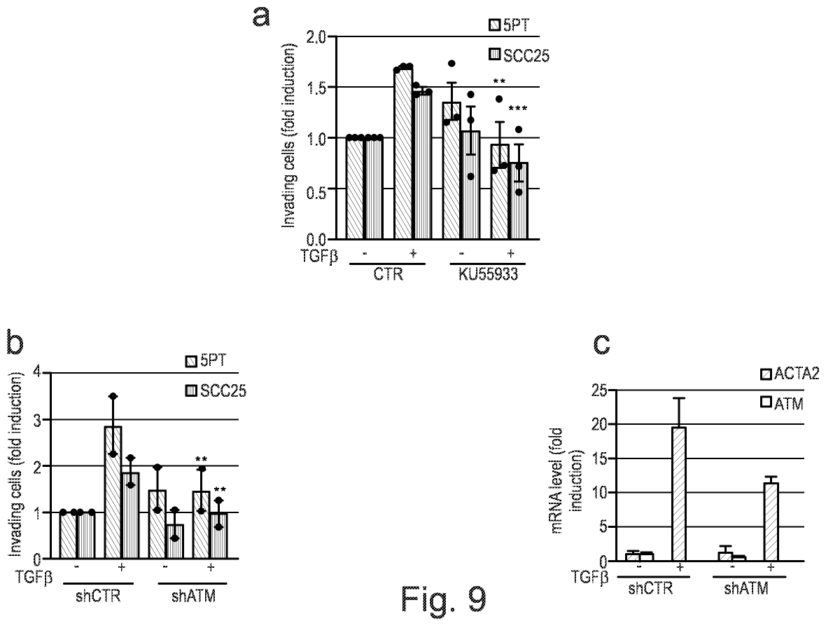

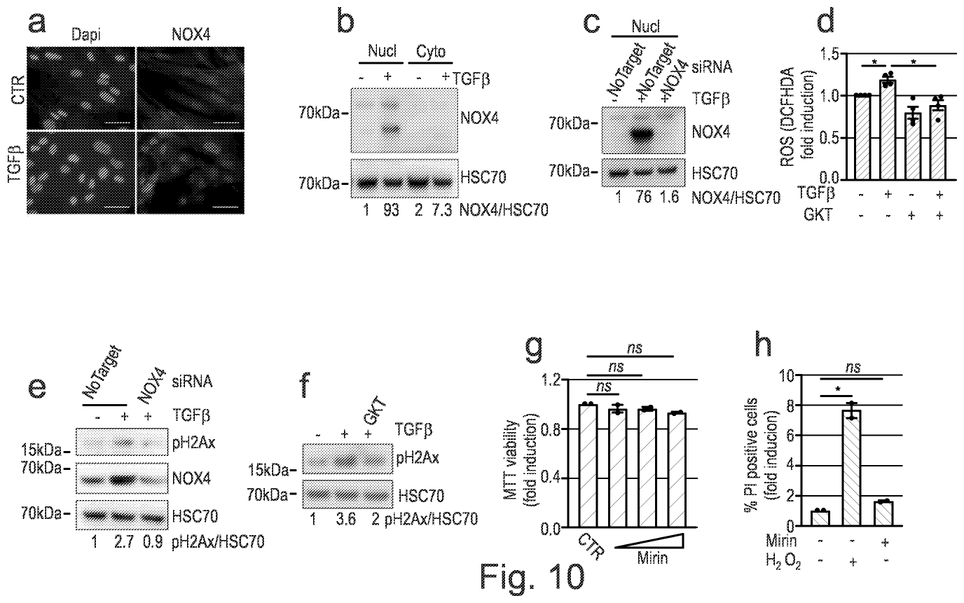

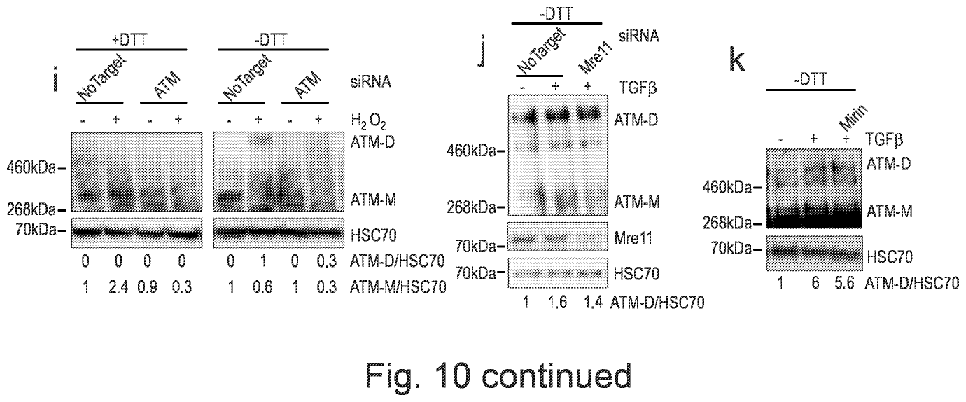

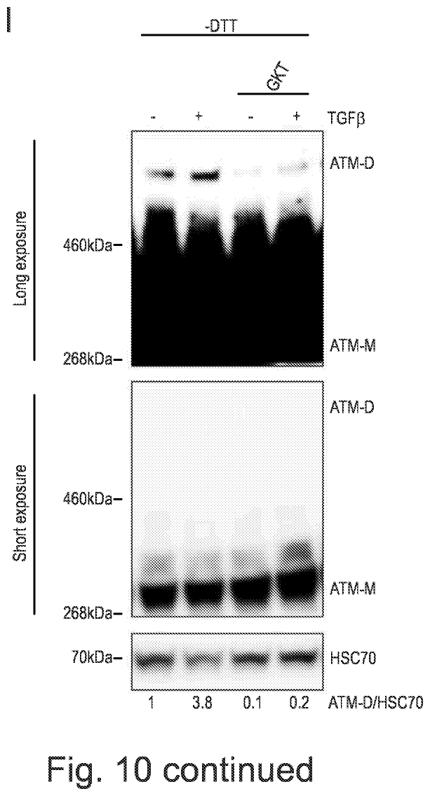

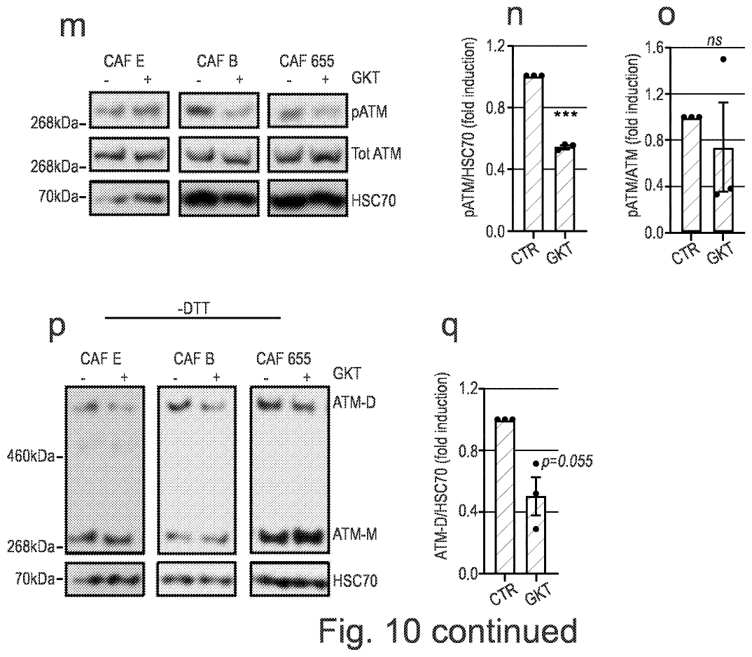

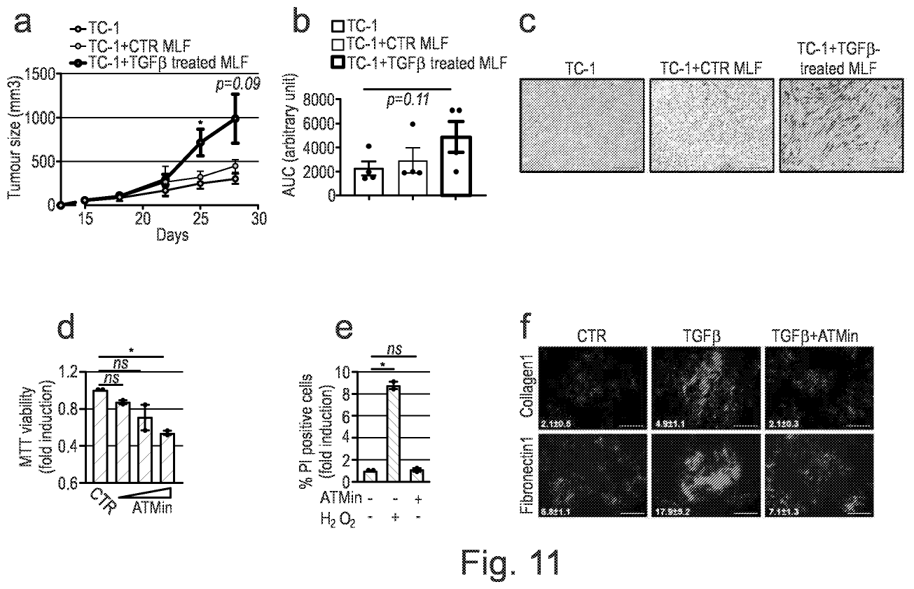

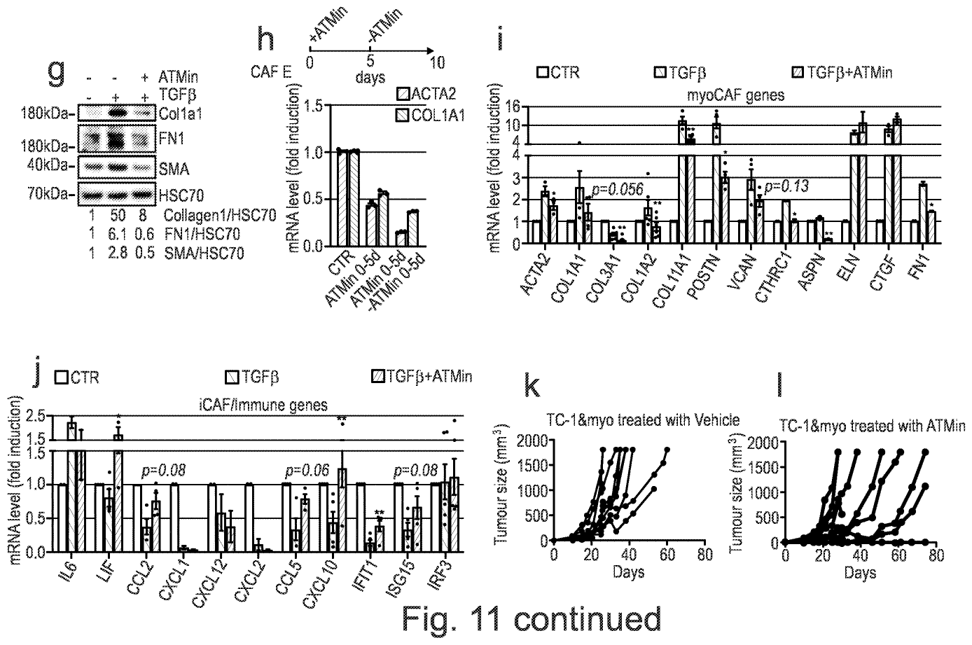

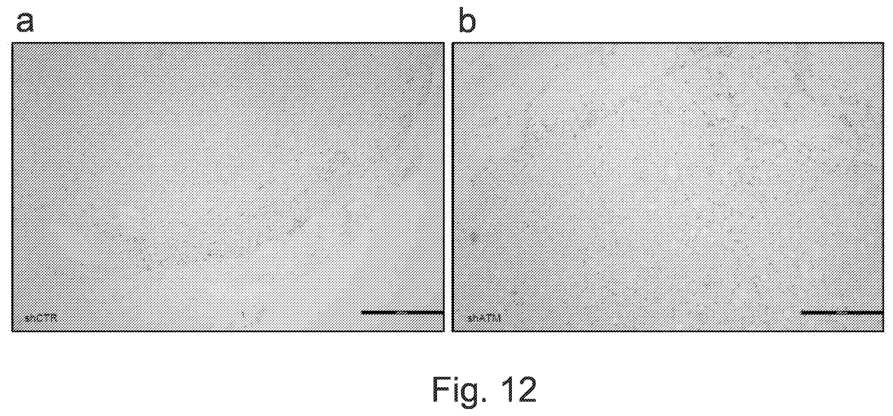

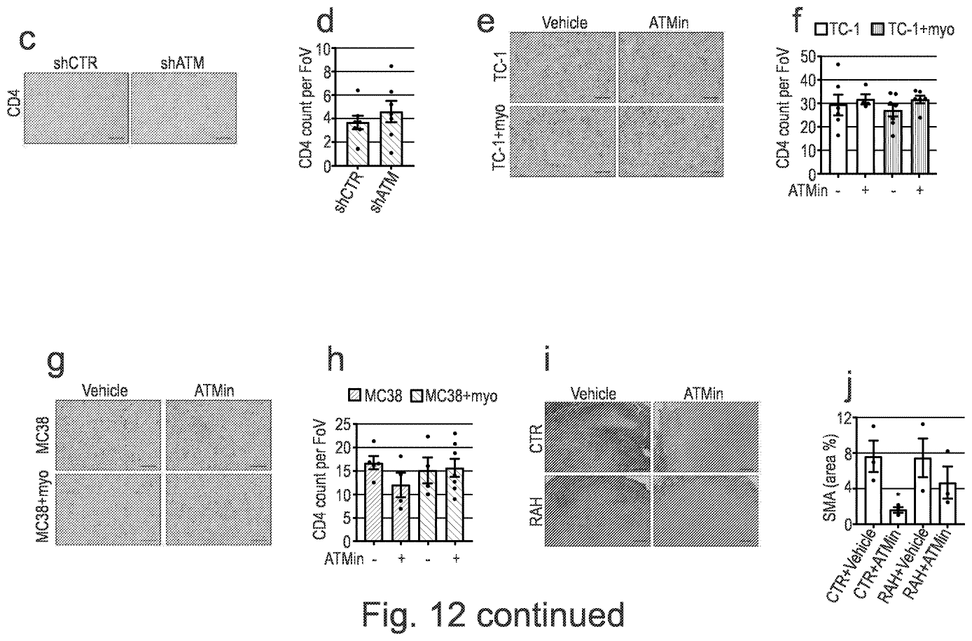

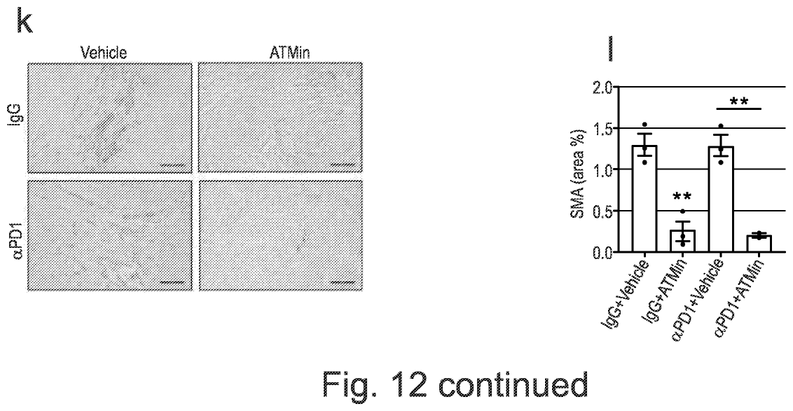

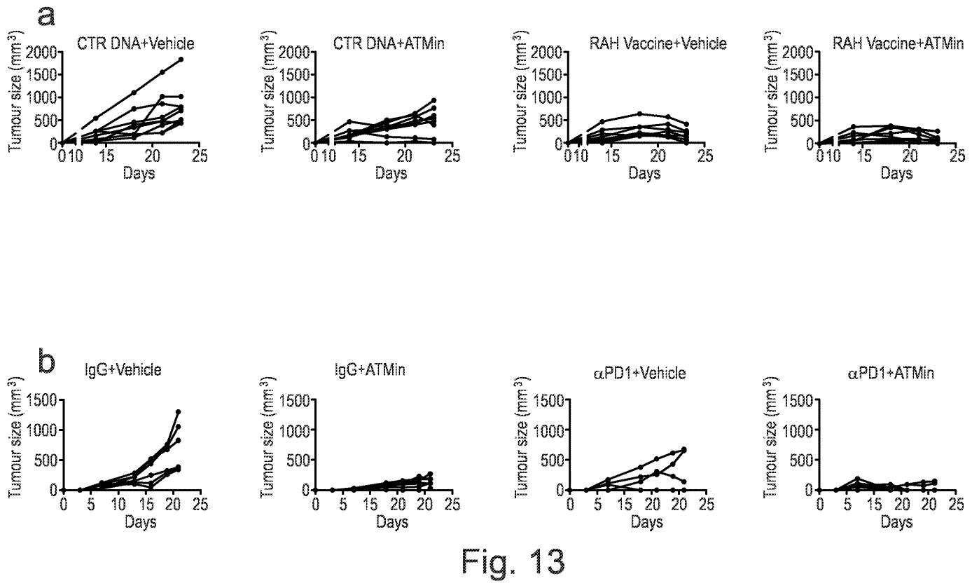

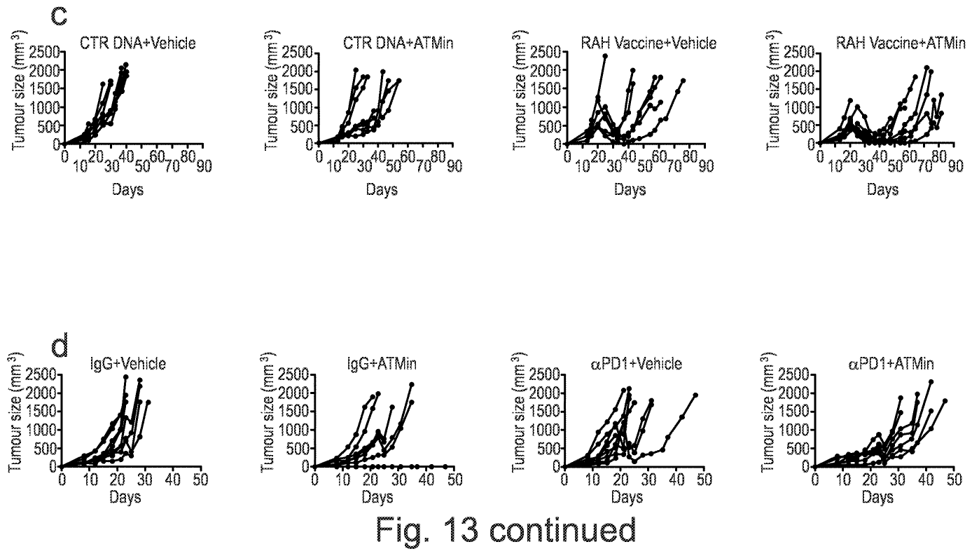

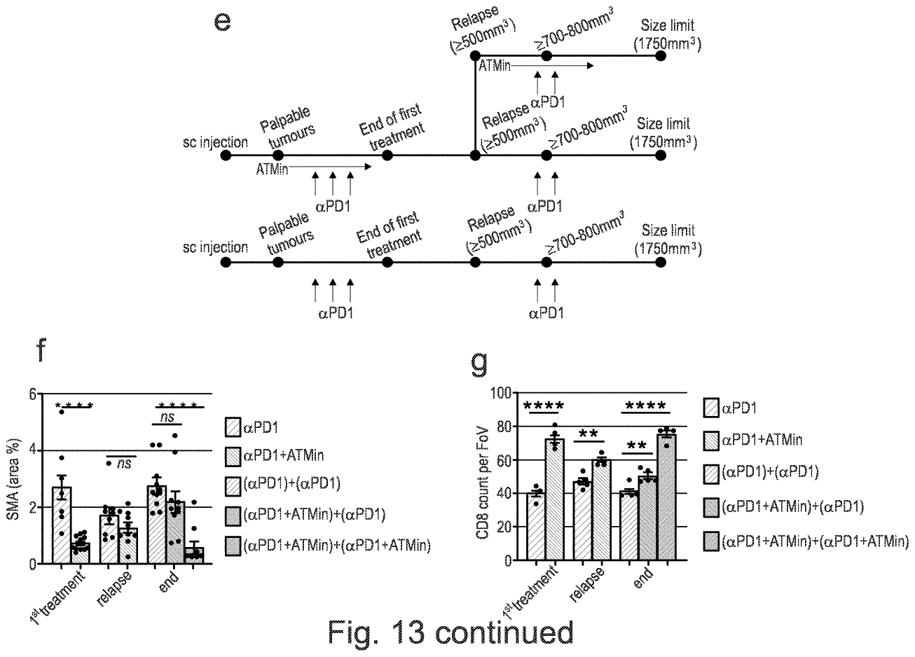

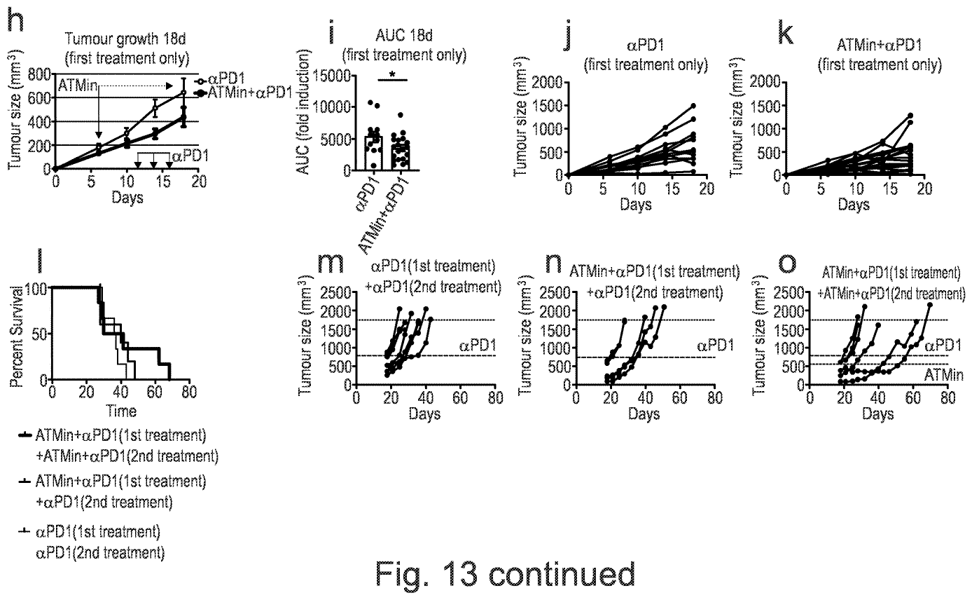

a,b) Western blotting of HFFF2 (a) and IMR90 (b) fibroblasts treated with TGF-β1 over time. c) Quantification of the proteins shown in the Western blotting from FIG. 7a,b, and FIG. 1a (n_{br}=3; Kruskall-Wallis statistical test is used). d) Q-RT-PCR of HFFF2 treated with TGF-β1 for 72 hours (nₜᵣ=2-3) showing ATM mRNA is not modulated by TGF-β1. e) Western blotting of HFFF2 treated with TGF-β1 over time. The single cropped bands for ATR or pATR belong to the same gel and image of the rest of the blot. f-i) Representative image of NSCLC MxlHC; bright field image of cytokeratin staining (scale bar=5 mm f); pseudo-colored images (scale bar=100 μm g; scale bar=20 μm h-i); single-stained and merged pseudo-colored images with examples of the cell regions used for the quantification highlighted for pATM or SMA positivity (subtracted CD31 staining labelled also). Heteroscedastic Student's t-test is used in the figure and refers to the control unless otherwise stated.

FIG. 8: ATM inhibition suppresses myofibroblast differentiation.

a) MTT assay of HFFF2 treated with 13.3 μM, 20 μM or 40 μM KU55933 for 72 hours (nₜᵣ=2).

b) PI assay of HFFF2 treated with 13.3 μM KU55933 for 72 hours or with H₂O₂(as positive control; nₜᵣ=2). c-g) Western blotting of human primary oral, colon and skin fibroblasts isolated from healthy donors treated with KU55933±TGF-β1 for 72 hours (densitometry analysis shown in FIG. 2c). h) Representative Q-RT-PCR of IMR90 fibroblasts treated as in c-g (n_{br}=1; nₜᵣ=2 shown in the plot). i) Representative immunofluorescence staining of collagen1 and relative quantification in mouse lung fibroblasts (MLF) treated with KU55933±TGF-β1 for one week (scale bar=500 μm). j-l) Q-RT-PCR of fibroblasts transfected with ATM siRNA (j; nₜᵣ=2), or stably expressing ATM shRNA (k,l) and treated as in c-g (nₜᵣ=3). m-o) Western blotting/quantification (m,n) and Q-RT-PCR (o; nₜᵣ=3) of HFFF2 stably expressing wild type (wt) or mutant ATM (mut) and treated as in c-g. p-s) Q-RT-PCR of HFFF2 treated with KU55933±TGF-β1 or 10 ng/ml IL-1p for 72 hours (nₜᵣ=3). t) MTT assay of HFFF2 treated with 1.5 μM, 3 μM, 10 μM CHK2 inhibitor CCT241533 (nₜᵣ=2) for 72 hours. u) PI assay of HFFF2 treated with 1.5 μM CHK2 inhibitor for 72 hours or H₂O₂ as positive control (nₜᵣ=2). Heteroscedastic Student T-test is used in the figure and it refers to TGF-β1-treated samples unless otherwise highlighted.

FIG. 9: ATM inhibition reverses the myofibroblast CAF phenotype and inhibits function.

a,b) Transwell invasion assays of 5PT or SCC25 cells towards conditioned media generated from HFFF2 treated with TGF-β1+/−KU55933 for 7 days (nₜᵣ=2-4 for 5PT and SCC25). c) Representative Q-RT-PCR of HFFF2 in (b) showing ATM and ACTA2 mRNA expression prior to the invasion assay (n_{br}=1, nₜᵣ=3; Standard deviation is shown). Two-way Anova is shown in the figure and refers to the TGF-β1 treated samples.

FIG. 10: TGFβ activates ATM via NOX4-driven DNA damage/MRN complex and oxidation.

a) Representative immunofluorescence staining of HFFF2 treated with TGF-β1 for 24 hours showing nuclear NOX4. Dapi used as nuclear counter-stain (scale bar=50 μm). b,c) Western blotting/quantification of nuclear/cytoplasm (a) or nuclear (b) extracts of HFFF2 transfected with NOX4 siRNA and treated for 48 hours with TGF-β1 showing nuclear NOX4. d-f) HFFF2 were treated for 48 hours with TGF-β1. ROS assay showing the DCFH-DA mean fluorescence of HFFF2 treated with GKT137831 (40 mM; nₜᵣ=2-3; d); Western blotting/quantification of HFFF2 transfected with NOX4 siRNA (e) or treated with GKT137831 (f). g) MTT assay of HFFF2 treated with 8 μM, 40 μM or 200 μM Mirin for 72 hours (nₜᵣ=2). h) PI assay of HFFF2 treated with 40 μM Mirin for 72 hours or with H₂O₂(as positive control; nₜᵣ=2). i) Western blotting/quantification of HFFF2 transfected with the indicated siRNAs and treated for 60 minutes with 2 mM H₂O₂(left and right gel run in reducing and non-reducing conditions respectively; Dithiothreitol=DTT). j-l) Non-reducing Western blotting/quantification (−DTT) of HFFF2 treated for 48 hours with TGF-β1 and as indicated. m-q) Western blotting [(reducing (m) and non-reducing (p)] and their quantification (n,o,q) of nuclear extracts from two HNSCC CAF and one NSCLC CAF (E, B & 655 respectively) treated daily with GKT137831 for 72 hours. Heteroscedastic Student's T-test is used throughout the figure.

FIG. 11: Targeting ATM in myofibroblasts reduces myoCAF gene expression, their intratumoural accumulation and inhibits tumour growth.

a-c) Mice were injected with TC-1+/−untreated (CTR) or TGF-β1-treated MLF (myoMLF; mouse number=4); tumour growth curves (a; the Homoscedastic Student's T-tests shown are relative to the comparison between the tumour size means in the latest two time points of TC-1 vs TC1+TGF-β1-treated MLF samples) AUC analysis (b) representative SMA IHC stainings (c) of the mouse tumours from a single experiment (scale bar=100 μm). d) MTT assay of HFFF2 treated with TGF-β1 for 72 hours+/−AZD0156 (0.5 μM, 1.25 μM, 2.5 μM; nₜᵣ=4). e) PI assay of HFFF2 treated for 72 hours with 0.5 μM AZD0156 or with H₂O₂(as positive control; nₜᵣ=2). f) Representative immunofluorescent staining for Collagen1 and Fibronectin and relative quantification of the mean in MLF treated for 7 days with TGF-β1+/−AZD0156 (0.5 μM; scale bar=500 μm; FoV=10). g) Western blotting/quantification of MLF treated with TGF-β1+/−AZD0156 (0.5 μM) for 72 hours. h) Q-RT-PCR of CAF E treated with AZD0156 for 5 days and then left them grow for further 5 days without AZD0156 (nₜᵣ=3 shown in the plot). i,j) Q-RT-PCR of MLF treated with TGF-β1+/−AZD0156 (0.5 μM) for a week (n_{br}=2-7; nₜᵣ=3). k,l) Individual mouse tumour volume measurements relative to FIG. 50. Mice were injected with TC-1+myoMLF and either treated with vehicle (mouse number=11; k) or treated with AZD0156 (mouse number=12; l). Heteroscedastic Student's T-test is used throughout the figure unless differently highlighted.

FIG. 12: Targeting ATM in myofibroblasts promotes tumour CD8 T-cell infiltration.

a,b) Representative images of CD8 IHC staining (at low magnification) in TC-1 myorich tumours (in FIG. 6a, 5g) with shCTR or shATM knockdown in myoMLF (scale bars=500 μm). c,d) Representative images of IHC staining (c) and relative quantification (d) of CD4 T-cells in the core of tumours described in FIG. 5g (nₜᵣ=FoV=5-10). e,f) Representative IHC staining (e) and relative quantification (f) of CD4 T-cells in the core of tumours described in FIG. 5k (nₜᵣ=FoV=5-10). g,h) Representative IHC staining (g) and relative quantification (h) of CD4 T-cells in the core of the tumours described in FIG. 5p (nₜᵣ=FoV=10). i,j) Representative images of SMA IHC staining (i) and relative quantification (j) of the mouse tumours described in FIG. 6i (nₜᵣ=FoV=3). k,l) Representative images of SMA IHC staining (k) and relative quantification (l) of the mouse tumours described in FIG. 6m (nₜᵣ=FoV=3). All scale bars=200 μm and the homoscedastic Student T-test is relative to control unless differently highlighted.

FIG. 13: Targeting ATM in myofibroblasts potentiates immunotherapy.

a) Individual mouse tumour volume measurements relative to FIG. 6i. b) Individual mouse tumour volume measurements relative to FIG. 6m. c) Individual mouse tumour volume measurements relative FIG. 6q. d) Individual mouse tumour volume measurements relative to FIG. 6r. e-o) Mice were injected with MC38+myoMCF and treated with αPD-1+/−AZD0156 for 10 days (first treatment; day 6-16). The second treatment with AZD0156 was given at relapse (size≥500 mm³) followed by two doses of αPD-1 (size 700/800 mm³)(schematic of the experiment shown in e). SMA (f; FoV=nₜᵣ=8-11) and CD8 (g; FoV=nₜᵣ=4-5) quantification of IHC staining (not shown) of single tumours from mice after the first treatment, at relapse or at the end of the experiment [at tumour size limit=1750 mm³ (top dotted line in m-o)]. The tumour growth curves (h), the AUC plot (i) and the single tumour growth curves (j,k) are relative to the earliest part of the experiment when all the mice received the first treatment (homoscedastic Student T-test is shown in i). Overall survival analysis (l) and single tumour growth curves (m-o) are relative to all the mice which relapsed after the first treatment (tumour size≥500 mm³) and received the second treatment (mouse number=5-7; Mantel-Cox log-rank test is not significant).

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

The present invention relates to inhibitors for use in treating solid tumours in a subject. In particular, the present invention relates to ATM inhibitors and/or CHK2 inhibitors for use in treating a myoCAF-high or -moderate tumour.

The inventors have surprisingly identified that ATM and/or CHK2 inhibition reversed myoCAF differentiation and potentiated immunotherapy response in myoCAF-high or -moderate tumours without affecting control (CAF-low) tumours comprising TC-1 and MC38 cells alone.

ATM signaling supports the differentiation of myoCAFs to suppress T-cell infiltration and antitumour immunity. Inhibiting ATM or CHK2 reverses myoCAF differentiation and therefore potentiates immunotherapy response. Accordingly, ATM and/or CH K2 qualify as novel targets to block myoCAF differentiation and make myoCAF-high and myoCAF-moderate solid tumours more susceptible to immunotherapy.

### General Definitions

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Features, integers, characteristics, compounds, chemical moieties or groups described in conjunction with a particular aspect, embodiment or example of the invention are to be understood to be applicable to any other aspect, embodiment or example described herein unless incompatible therewith.

As understood by a person skilled in the medical art, the terms, “treat” and “treatment,” refer to medical management of a disease, disorder, or condition of a subject (i.e., patient) (see, e.g., Stedman's Medical Dictionary). In general, an appropriate dose and treatment regimen provide the inhibitor in an amount sufficient to provide therapeutic and/or prophylactic benefit. Therapeutic benefit for subjects to whom the inhibitor described herein is administered, includes, for example, an improved clinical outcome, wherein the object is to prevent or slow or retard (lessen) an undesired physiological change associated with the disease, or to prevent or slow or retard (lessen) the expansion or severity of such disease. As discussed herein, effectiveness of the inhibitor may include beneficial or desired clinical results that comprise, but are not limited to, abatement, lessening, or alleviation of symptoms that result from or are associated with the disease to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival.

As used herein and in the art, the terms “cancer” or “tumour” are clinically descriptive terms that encompass diseases typically characterized by cells exhibiting abnormal cellular proliferation. The term cancer is generally used to describe a malignant tumour or the disease state arising from the tumour. Alternatively, an abnormal growth may be referred to in the art as a neoplasm. The term tumour, such as in reference to a tissue, generally refers to any abnormal tissue growth that is characterized, at least in part, by excessive and abnormal cellular proliferation. A tumour may be metastatic and capable of spreading beyond its anatomical site of origin and initial colonization to other areas throughout the body of the subject. A cancer may comprise a solid tumour or may comprise a “liquid” tumour (e.g., leukemia and other blood cancers). As used herein, the term “solid tumour” refers to an abnormal mass of tissue that usually does not contain cysts or liquid areas. Solid tumours may be benign (not cancer), or malignant (cancer). Different types of solid tumours are named for the type of cells that form them. Examples of solid tumours are sarcomas, carcinomas, and lymphomas. Leukemias (cancers of the blood) generally do not form solid tumours.

The term “ATM” is the abbreviation for “ataxia telangiectasia mutated”. ATM is a DNA damage-inducible protein kinase, which phosphorylates a plethora of substrates that participate in DNA damage response. ATM phosphorylates several key proteins that initiate activation of the DNA damage checkpoint, leading to cell cycle arrest, DNA repair or apoptosis. Several of these targets, including p53, CHK2, BRCA1, NBS1 and H2AX are tumour suppressors. ATM belongs to a family surveillance kinases known as phosphatidylinositol 3-kinase-related protein kinases (PIKKs). Members of this kinase family also include ATR, the DNA-dependent protein kinase catalytic subunit (DNA-PKcs), mTOR, SMG1 and TRRAP8,9. ATM, ATR and DNA-PKcs monitor the cell for DNA damage by sensing DSBs (ATM and DNA-PKcs) or single-stranded DNA (ssDNA; ATR). The UniProt accession number for human ATM is Q13315.

The term “CHK2” is the abbreviation for “checkpoint kinase 2”. CHK2 is a serine-threonine kinase. CHK2 is involved in DNA repair, cell cycle arrest or apoptosis in response to DNA damage. Mutations to the CHEK2 gene have been linked to a wide range of cancers. CHK2 is a downstream target of ATM. The UniProt accession number for human CHK2 is 096017.

As used herein, the term “myofibroblastic cancer-associated fibroblast” or “myoCAF” refers to activated fibroblasts that are analogous to myofibroblasts found in wound-healing and tissue fibrosis. These contractile, secretory cells are regulated through TGFβ1 signalling, express α-smooth muscle actin (SMA), and secrete collagen-rich extracellular matrix (ECM). A cancer-associated fibroblast (CAF) (also known as tumour-associated fibroblast; carcinogenic-associated fibroblast; activated fibroblast, peritumoral fibroblast) is a cell type within the tumour microenvironment that promotes tumourigenic features by exerting tissue tension, remodelling the extracellular matrix, and by secreting cytokines, growth factors and chemokines. TGFb/SMAD signaling is recognized as the central pathway regulating myofibroblast/myoCAF differentiation, although other pathways also influence the myoCAF phenotype. It has been found previously that during TGFb1-induced differentiation, myofibroblasts upregulate a number of genes associated with DNA repair, suggesting that the DNA damage response (DDR) pathway may be triggered during the process. The DDR is a complex cellular network of coordinated pathways that maintain genome integrity by detecting and repairing DNA lesions, orchestrated by DNA damage-sensing kinases ataxia-telangiectasia mutated (ATM), ataxia-telangiectasia mutated and Rad3-related (ATR), or DNA-dependent protein kinase (DNAPKc). TGFb/SMAD signaling has been reported to contribute to the cellular DDR induced by genotoxic insults or by radiation/stress-driven bystander effects. However, it remains to be determined if and how the TGFb/SMAD pathway affects DNA repair during the acquisition of the myofibroblast/myoCAF phenotype. The myofibroblast differentiation process is associated with the generation of intracellular reactive oxygen species (ROS), and it previously has been described a role for the ROS-generating enzyme NADPH oxidase 4 (NOX4) in promoting and maintaining the myoCAF phenotype.

Herein the terms “diseases”, “disorders”, and “conditions” are used interchangeably and refer to a disorder of structure or function in a human or animal, especially one that produces specific symptoms or that affects a specific location and is not simply a direct result of physical injury. Diseases that can be treated and/or prevented with the inhibitor described herein are described elsewhere herein in detail.

As used herein the term “subject” refers to an individual, e.g., a human, dog, cat, pig, horse, mouse, cow, rat etc having or at risk of having a specified condition, disorder or symptom. The subject may be a patient i.e., a subject in need of treatment in accordance with the invention. The subject may have received treatment for the condition, disorder or symptom. Alternatively, the subject has not been treated prior to treatment in accordance with the present invention. The subject is preferably in need of administration of an inhibitor of the invention. Preferably, the subject is human.

As used herein, the “administration” or “administering” of a (pharmaceutical) composition described herein to a subject includes any route of introducing or delivering to a subject which allows for the composition to perform its intended function. Administration can be carried out by any suitable route, including orally, intranasally, intraocularly, ophthalmically, parenterally (intravenously, intramuscularly, intraperitoneally, or subcutaneously), or topically. Administration includes self-administration and the administration by another. The composition can be administered as a therapeutically effective amount.

As used herein, the phrase “therapeutically effective amount” means a dose or concentration (e.g. plasma concentration) in a subject that provides the specific pharmacological effect for which the described inhibitors or compositions are administered, e.g., to treat a disease of interest in a target subject. The therapeutically effective amount may vary based on the route of administration and dosage form, the age and weight of the subject, and/or the disease or condition being treated.

A composition may be a pharmaceutical composition or formulation that comprises the inhibitor and a pharmaceutically acceptable excipient, adjuvant, diluent and/or carrier. Pharmaceutical compositions or formulations may routinely contain pharmaceutically acceptable concentrations of salt, buffering agents, preservatives, compatible carriers, supplementary immune potentiating agents such as adjuvants and cytokines and optionally other therapeutic agents or compounds.

As used herein, “pharmaceutically acceptable” refers to a material that is not biologically or otherwise undesirable, i.e., the material may be administered to an individual along with the selected inhibitor without causing any undesirable biological effects or interacting in a deleterious manner with any of the other components of the pharmaceutical composition in which it is contained.

Excipients are natural or synthetic substances formulated alongside an active ingredient (e.g. an inhibitor as provided herein), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well known in the art and include cell-penetrating peptides. A suitable adjuvant is therefore easily identifiable by one of ordinary skill in the art.

Diluents are diluting agents. Pharmaceutically acceptable diluents are well known in the art and include water or saline. A suitable diluent is therefore easily identifiable by one of ordinary skill in the art.

Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art and include serum albumin. A suitable carrier is therefore easily identifiable by one of ordinary skill in the art.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. It is to be understood that this invention is not limited to the particular methodology, protocols, and reagents described, as these may vary, depending upon the context they are used by those of skill in the art.

### Monotherapy

According to a first aspect of the invention a method of treating a solid tumour in a subject is provided, the method comprising administering a therapeutically effective amount of an ATM inhibitor and/or a CHK2 inhibitor to the subject, wherein the solid tumour is a myoCAF-high or -moderate tumour.

The inhibition of ATM and/or CHK2 suppresses and reverses myofibroblast accumulation, slows down tumour growth and improves overall survival (see Examples section). Overall, this demonstrates that ATM and/or CHK2 can be targeted to suppress intratumoural myoCAF accumulation, resulting in reduced solid tumour growth.

According to another aspect the invention a method of treating a solid tumour in a subject is provided, the method comprising:
a) identifying a subject as having a solid tumour characterized as a myoCAF-high or -moderate tumour
b) administering to the subject a therapeutically effective amount of an ATM inhibitor.

The term “myoCAF-high tumour” or “myoCAF-moderate tumour” relates to a solid tumour wherein a α-smooth muscle actin (αSMA) staining of a tumour tissue sample shows a stromal positivity of more than 50% (myoCAF-high); or wherein a α-smooth muscle actin (αSMA) staining of a tumour tissue sample shows a stromal positivity of 5-50% (myoCAF-moderate), respectively. In other words, tumour samples wherein 5-50% of the stroma in the sample has detectable α-smooth muscle actin (αSMA) staining are defined herein as myoCAF-moderate tumour samples (such that the tumour that the sample has been collected from may be referred to as a myoCAF-moderate tumour). Similarly, tumour samples wherein more than 50% of the stroma in the sample has detectable α-smooth muscle actin (SMA) staining are defined herein as myoCAF-high tumour samples (such that the tumour that the sample has been collected from may be referred to as a myoCAF-high tumour). Immunohistochemistry for αSMA is standardly used in clinical pathology laboratories for diagnostic purposes. Several other methods for staining a tumour sample for αSMA are known in the art. For further details regarding αSMA staining please see Examples section.

The terms “myoCAF-high tumour”, “myoCAF-rich tumour” and “tumour with high levels of myoCAF” are used interchangeably herein.

The terms “myoCAF-moderate tumour”, and “tumour with moderate levels of myoCAF” are used interchangeably herein.The terms “SMA” and “αSMA” are used interchangeably herein.

In one example, the subject has been identified as having a myofibroblastic cancer-associated fibroblast (myoCAF)-high or -moderate solid tumour.

In this example, the method according to the invention may therefore comprise the step of assessing whether the tumour of the subject to be treated is a myoCAF-high or -moderate tumour. This may be assessed using any appropriate method for determining the presence of αSMA in the tumour stroma. For example, this may be assessed by performing immunohistochemistry αSMA staining on a tumour tissue sample from that subject e.g. using the methods described elsewhere herein. When αSMA staining of the tumour tissue sample reveals a stromal positivity of 5% or more, the tumour may be identified as being eligible for treatment using one of the methods described herein.

In one example, the solid tumour is a cancer selected from the group consisting of: non-small cell lung cancer (NSCLC), head and neck squamous cell cancer (HNSCC), esophageal cancer, ovarian cancer, colorectal cancer, liver cancer, breast cancer, ovarian cancer, cervical cancer, endometrial cancer, melanoma, hepatocellular carcinoma, kidney cancer, prostate cancer, gastric cancer, pancreatic cancer, urinary bladder cancer, and brain cancer; optionally wherein the brain cancer is glioblastoma. In a particular example, the solid tumour is a cancer selected from NSCLC or HNSCC.

The term “inhibitor” as used herein refers to any compound that reduces, abolishes, prevents, blocks, suppress, slows, or interferes the functionality of ATM and/or CHK2, either directly or indirectly. Several inhibitors of ATM and/or CHK2 are known. Suitable examples are listed elsewhere herein. Inhibition may be reversible or irreversible. Inhibitory function of inhibitors investigated by testing kinase phosphorylation (eg Western blotting). Such techniques are well known in the art.

As would be clear to a person skilled in the art, an inhibitor may function at the level of the target gene, transcript or protein. In some examples, the inhibitor may reduce ATM and/or CHK2 activity by at least 5%, 10%, 15%, 20%, 25%, 30%, 35%, 40%, 45%, 50%, 55%, 60%, 65%, 70%, 75%, 80%, 85%, 90%, 95%, or 100% as compared to the activity of a control (e.g., activity in the absence of the inhibitor).

An inhibitor can be an agent (e.g. an inhibitory nucleic acid, a binding molecule (e.g. a small molecule or an antibody), or peptide) that inhibits ATM and/or CHK2 function. A person of skill in the art will be able to readily identify suitable ATM and/or CHK2 inhibitors using known methods in the art (e.g. based on assaying the effect of potential inhibitors on known ATM and/or CHK2 activity).

In some examples, the inhibitor inhibits, reduces, slows, halts, blocks, suppresses, abolishes and/or prevents ATM activity. In another example, the inhibitor inhibits, reduces, slows, halts, blocks, suppresses, abolishes and/or prevents the CHK2 activity. In some examples the inhibitor inhibits, reduces, slows, halts, blocks, suppresses, abolishes and/or prevents ATM activity and CHK2 activity.

In a particular example, the inhibitor is a direct ATM inhibitor. In another example, the inhibitor is a direct CHK2 inhibitor. A “direct inhibitor”, as used herein, refers to an inhibitor that directly targets the target gene, transcript or protein. In the context of ATM, a direct inhibitor directly inhibits the ATM protein, a ATM transcript and/or the ATM gene, thereby reducing ATM protein activity and/or expression. In the context of CHK2, a direct inhibitor directly inhibits the CHK2 protein, a CHK2 transcript and/or the CHK2 gene, thereby reducing CHK2 protein activity and/or expression.

In some examples, the inhibitor may, besides inhibiting ATM and/or CHK2 inhibit other cellular components (e.g. inhibit the activity of other proteins) as well. Any inhibitor that inhibits, reduces, slows, halts, blocks, suppresses, abolishes and/or prevents ATM and/or CHK2 activity, (irrespective of whether it also has inhibitory effects on other cellular components) falls under the definition of the term “inhibitor” as used herein. For example, the inhibitor might inhibit the function of CHK1 and also inhibit the function of CHK2. It would still be considered as a CHK2 inhibitor according to its use herein.

In some examples, the inhibitor may be a direct inhibitor of ATM (and at the same time be an indirect inhibitor of CHK2). In some examples, the inhibitor may be a direct inhibitor of CHK2 (and at the same time be an indirect inhibitor of ATM).

Accordingly, in some examples, the inhibitor directly targets the ATM gene, ATM transcript or protein. For example, the inhibitor may directly bind to the ATM gene, ATM transcript or protein. In some examples, the inhibitor directly targets the CHK2 gene, RNA transcript or protein. For example, the inhibitor may directly bind to the CHK2 gene, RNA transcript or protein.

Any suitable ATM inhibitor and/or CHK2 inhibitor may be used. In a particular example, the ATM inhibitor and/or CHK2 inhibitor may be selected from the group consisting of: an inhibitory nucleic acid, a binding molecule (e.g. a small molecule or an antibody (including functional fragments thereof)) and a peptide. Suitable examples of inhibitory nucleic acids, binding molecules (e.g. small molecules, or antibodies (including functional fragments thereof)) or peptides would be readily identifiable to a person of skill in the art.

In some examples, the inhibitor may be an “inhibitory nucleic acid” whose presence in a cell causes the degradation of or inhibits the function, transcription, or translation of its target gene in a sequence -specific manner. Exemplary inhibitory nucleic acids include aptamers, siRNA, microRNA-adapted shRNA, shRNA, precursor microRNA (pre-miRNA), pri-miRNA, miRNA, amiRNA, interfering RNA or RNAi, dsRNA, ribozymes, antisense oligonucleotides (ASO), and DNA expression cassettes encoding said inhibitory nucleic acids.

In some examples, the inhibitor is a binding molecule. Preferably, the binding molecule binds to ATM and/or CHK2 and inhibits its activity (for example it inhibits one or more of the activities for ATM and/or CHK2 described elsewhere herein).

An example of a binding molecule is a small molecule. Binding molecules also include antibodies as well as non-immunoglobulin binding agents, such as phage display-derived peptide binders, and antibody mimics, e.g., affibodies, tetranectins (CTLDs), adnectins (monobodies), anticalins, DARPins (ankyrins), avimers, iMabs, microbodies, peptide aptamers, Kunitz domains, aptamers and affilins. The term “antibody” includes, for example, both naturally occurring and non-naturally occurring antibodies, polyclonal and monoclonal antibodies, chimeric antibodies and wholly synthetic antibodies and fragments thereof, such as, for example, the Fab′, F(ab′)₂, Fv or Fab fragments, or other antigen recognizing immunoglobulin fragments. Antibodies which bind a particular epitope can be generated by methods known in the art. For example, polyclonal antibodies can be made by the conventional method of immunizing a mammal (e.g., rabbits, mice, rats, sheep, goats). Polyclonal antibodies are then contained in the sera of the immunized animals and can be isolated using standard procedures (e.g., affinity chromatography, immunoprecipitation, size exclusion chromatography, and ion exchange chromatography). Monoclonal antibodies can be made by the conventional method of immunization of a mammal, followed by isolation of plasma B cells producing the monoclonal antibodies of interest and fusion with a myeloma cell (see, e.g., Mishell, et al., 1980). Screening for recognition of the epitope can be performed using standard immunoassay methods including ELISA techniques, radioimmunoassays, immunofluorescence, immunohistochemistry, and Western blotting. In vitro methods of antibody selection, such as antibody phage display, may also be used to generate antibodies. Preferably, a nuclear localization signal is added to the antibody in order to increase localization to the nucleus.

In some examples, the activity and/or expression of a target protein (e.g. ATM or CHK2) in a cell may be inhibited, reduced, slowed, halted, blocked, suppressed, abolished and/or prevented by introduction of a mutation that disrupts the target gene (e.g. by introduction of a mutation that disrupts the ATM gene or the CHK2 gene). As would be clear to a skilled person, such a mutation may decrease expression of the protein encoded by the target gene (e.g. ATM or CHK2), abrogate expression of the gene entirely, or render the gene product non-functional. Accordingly, in some examples, the mutation may be a loss of function mutation. In some examples, the mutation may be a point mutation, an insertion, a substitution or a deletion. In some examples, both alleles of the target gene are mutated. In some examples, the mutation may be located in a coding region (e.g., in an exon of ATM or CHK2) and/or in a non-coding region of the target gene (e.g. in the promoter region of ATM or CHK2).

Mutation of the ATM and/or CHK2 gene may be accomplished by methods well known in the art, including gene editing techniques. For example, a mutation may be introduced into the ATM and/or CHK2 gene using a targeted genome editing technique (e.g. using targeted genome editing construct(s)). For example, a mutation may be introduced into the ATM and/or CHK2 gene using zinc-finger nucleases (ZFNs), transcription activator-like effector nucleases (TALENs), clustered regularly interspaced short palindromic repeat (CRISPR) nucleases (e.g. RNA-guided DNA endonuclease Cas9 (or variants thereof)) or meganucleases.

The CHK2 inhibitor can either be a “pan-CHK inhibitor”, which is a selective inhibitor of both CHK1 and CHK2, or it can be a CHK2 specific inhibitor. A pan-CHK inhibitor can inhibit both isoforms with equal potency and no selective effects or it can inhibit both but with a higher potency for either CHK1 or CHK2. For example, a pan-CHK inhibitor with a higher potency for CHK1 can inhibit both isoforms but inhibits CHK1 with a higher efficiency. CCT241533, BML-277, PHI-101, VRX0466617, PV1019 are examples of selective CHK2 inhibitors. AZD7762, Y2606368 (Prexasertib) are examples of pan-CHK inhibitors.

In one example, the CHK inhibitor is a pan-CHK inhibitor, or a CHK2 inhibitor. In one example, the CHK inhibitor is a direct pan-CHK inhibitor or a direct CHK2 inhibitor.

In one example the ATM inhibitor directly inhibits ATM.

In one example the CHK2 inhibitor directly inhibits CHK2.

In one example, the inhibitor prevents or reverses the differentiation of myoCAFs. By preventing and/or reversing the differentiation of the myoCAFs, tumour growth can be reduced, slowed, myofibroblast accumulation can be suppressed and overall survival can be improved. Methods for determining if the differentiation of myoCAFs is prevented or reversed are well known in the art. For example, in vitro, fibroblasts are treated with TGF-b1 to induce myofibroblast differentiation resulting in cell contractility, formation of stress fibres, upregulation of SMA and ECM genes (eg., Col1A1, FN-EDA). Inhibitors are used to prevent or reverse this transdifferentiation.

In one example the ATM inhibitor inhibits ATM by blocking the kinase function of ATM. By inhibiting ATM's kinase function ATM can no longer phosphorylate the multitude of downstream transducers and effector proteins at SQ/TQ motifs. Target proteins include the DSB repair factor CtIP, histone variant H2AX, tumour suppressors BRCA1 and p53, and the checkpoint transducing kinase CHK2. Methods for determining if the kinase function of ATM has been blocked are well known in the art. Several inhibitors that inhibit ATM by blocking its kinase function are known. For example, the ATM inhibitor KU-55933 inhibits the kinase function of ATM by forming two distinct hydrogen bonds via the morpholine O atom with C2770 of the hinge region and via the pyran-4-one carbonyl O atom with the catalytically important residue K2717 in the N-lobe. The non-planar thianthrene ring is involved in extensive van der Waals (vdW) contacts with residues P2699 in the P-loop, L2715, L2767, W2769 in the N-lobe, P2775, L2877 and 12888 in the C-lobe and activation loop, respectively.

Another ATM inhibitor that inhibits ATM kinase function is M4076. M4076 inhibits the kinase function of ATM by binding in the ATM active site cleft. The 1,3-dihydro-imidazo[4,5-c]quinolin-2-one moiety binds towards the hinge region, indicating a classical hydrogen bond between the N5 atom and C2770, as well as non-classical hydrogen bonds of the 4-CH group to C2770 and T2773. Remarkably, the imidazolone ring forms extensive π-π and amide-π stacking interactions towards the W2769 side chain of the hinge region, as calculated from the atomic coordinates by the software tool ViewContacts57. The 7-methoxy group attached to the quinoline ring interacts with the gatekeeper residue L2767 and with L2715 via vdW contacts. The M4076 1,3-dimethyl-1H-pyrazole substituent is in vdW contact with 12888 next to the DLG motif of the activation loop and in hydrogen bond distance to K2717 via the unsubstituted N atom.

In one example, the CHK2 inhibitor inhibits CHK2 by blocking the kinase function of CHK2. Methods for determining if the kinase function of CHK2 has been blocked are well known in the art. Several inhibitors that inhibit CHK2 by blocking its kinase function are known. For example, the CHK2 inhibitor CCT241533 inhibits the kinase by occupying the ATP-binding site, sandwiched between the hydrophobic side chains of valine 234 and leucine 354. Similarly, BML-277, VRX0466617 and AZD7762 are ATP-competitive inhibitors of CHK2.

In one example, the ATM and/or the CHK2 inhibitor promotes intra-tumoral T-cell infiltration into the solid tumour. In particular, the ATM and/or CHK2 inhibitor promotes intra-tumoral CD8 T cell infiltration into the solid tumour. By promoting the infiltration of CD8 T cells into myoCAF-high/-moderate tumours the early response to immunotherapy can be potentiated.

In one example, the ATM and/or the CHEK2 inhibitor potentiates the solid tumour's response to anti-PD-1 blockade.

In one example, the ATM inhibitor is selected from the group consisting of KU-55933, KU-60019, KU-59403, CP-466722, AZ31/AZ32, AZD0156, and AZD1390. In one example the ATM inhibitor is AZD0156. In one example, the ATM inhibitor is KU-55933

In one example, the CHK2 inhibitor is selected from the group consisting of CCT241533, BML-277, PHI-101, VRX0466617, PV1019, AZD7762, and Y2606368 (Prexasertib).

An inhibitor of the invention can be for use or administration alone or in combination with at least one or more other compounds. Administration “in combination with” at least one or more other compounds includes simultaneous (concurrent) and consecutive administration in any order. “Combined use” and “combination” in the context of the invention also includes a pharmaceutical product comprising both the inhibitor and at least one or more other compounds, as discrete separate dosage forms, in separate containers or e. g. in blisters containing both types of drugs in discrete solid dosage units, e.g. in a form in which the dosage units which have to be taken together or which have to be taken within one day are grouped together in a manner which is convenient for the patient. Said pharmaceutical product itself or as a part of a kit may contain instructions for the simultaneous, sequential or separate administration of the discrete separate dosage units, to a subject in need thereof.

In one example, the ATM inhibitor and/or the CHK2 inhibitor is used in a neoadjuvant, adjuvant, first line and/or palliative manner.

When used in as a neoadjuvant, the ATM inhibitor and/or the CHK2 inhibitor are administered before the main treatment. For example, the main treatment can be radiotherapy, surgery, chemotherapy or immunotherapy.

When used in as an adjuvant, the ATM inhibitor and/or the CHK2 inhibitor is given in addition to the main treatment to maximize its effectiveness. In addition, “adjuvant” use of the ATM inhibitor and/or the CHK2 inhibitor can be after the main treatment to reduce the risk of recurrence. In this example, For example, the main treatment can be radiotherapy, surgery, chemotherapy or immunotherapy.

When used as a first line treatment, the ATM inhibitor and/or the CHK2 inhibitor is considered to be the main treatment, which might be accompanied by other treatments regimens.

When used as a palliative treatment, the ATM inhibitor and/or the CHK2 inhibitor are administered with the purpose of optimizing the quality of life and mitigating the suffering of the subject but not with the purpose of actually healing the cancer.

The ATM inhibitor and/or the CHK2 inhibitor may also be used in several different ways as part of one treatment regimen protocol. For example, the ATM inhibitor and/or the CHK2 inhibitor can be used as a neoadjuvant and subsequently as an adjuvant.

### Combinational Therapies

The inventors have identified that by combining an ATM inhibitor (and/or the CHK2 inhibitor) with an immunotherapeutic agent, CAF-mediated immunotherapy resistance of solid tumours can be overcome such that the inhibitor(s) potentiate(s) the response to immune checkpoint inhibitors and cancer vaccination. Hence by using the inhibitors described herein together with immunotherapeutic approaches a more successful treatment of solid tumours can be achieved.

The data provided herein indicates that targeting fibroblast ATM in vivo suppresses myoCAF-high tumour growth, promotes intratumoural CD8 T-cell infiltration, and potentiates the response to anti-PD-1 blockade and antitumour vaccination. ATM signaling supports the differentiation of myoCAFs to suppress T-cell infiltration and antitumour immunity, which demonstrates the clinical use of ATM inhibitors in combination with checkpoint inhibition in myoCAF-moderate or myoCAF-high, immune-cold tumours. Similarly, based on the data provided herein, CHK2 inhibitors may be used in combination with checkpoint inhibition to treat myo-CAF moderate or myoCAF-high, immune-cold tumours.

Therefore, according to another aspect of the invention a subject may be treated with a combination of an ATM inhibitor and/or the CHK2 inhibitor and a immunotherapeutic agent (wherein the ATM inhibitor and/or the CHK2 inhibitor and immunotherapeutic agent are either in separate/distinct compositions or are combined in the same composition, for example as a formulation described herein).

In another aspect, the present invention provides a method of treating a solid tumour in a subject, the method comprising administering a therapeutically effective amount of:
a) an ATM inhibitor and/or a CHK2 inhibitor; and
b) at least one immunotherapeutic agent;
   to the subject, wherein the inhibitor and the at least one immunotherapeutic agent are administered simultaneously, separately or consecutively, and wherein the solid tumour is a myoCAF-high or -moderate tumour.

In another aspect, the present invention provides a method of treating a solid tumour in a subject, the method comprising administering a therapeutically effective amount of an ATM inhibitor and/or a CHK2 inhibitor to the subject, wherein the subject is undergoing treatment with, has been treated with, or has been prescribed treatment with at least one immunotherapeutic agent, and wherein the solid tumour is a myoCAF-high or -moderate tumour.

In another aspect, the present invention provides a method of treating a solid tumour in a subject, the method comprising administering a therapeutically effective amount of at least one immunotherapeutic agent to the subject, wherein the subject is undergoing treatment with, has been treated with, or has been prescribed treatment with an ATM inhibitor and/or a CHK2 inhibitor, and wherein the solid tumour is a myoCAF-high or -moderate tumour.

As used herein, a subject that is “undergoing treatment” with a specified drug (e.g. an immunotherapeutic agent or an ATM and/or CHK2 inhibitor) means that the subject has already commenced treatment with the specified drug (the subject may be in any phase of the treatment e.g. induction phase, maintenance phase, recovery phase etc).

In another aspect, the present invention provides a method for promoting T-cell infiltration and antitumour immunity of a solid tumour in a subject, comprising subjecting the subject to a therapeutically effective amount of an ATM inhibitor and/or a CHK2 inhibitor in combination with at least one immunotherapeutic agent, wherein the solid tumour is a myoCAF-high or -moderate tumour.

The term “immunotherapeutic agent” as used herein refers to any agent that may be able to stimulate or suppress the immune system to help the body fight a disease or disorder. The immunotherapeutic agent can either target only certain cells, compounds, proteins of the immune system or it can affect the immune system in a general way. Examples of immunotherapeutic agents are cytokines, vaccines, antibodies, compounds, cells etc. The immunotherapeutic agent may be any kind of agent that can be used for any type of immunotherapy, such as:
a) Monoclonal antibodies
b) Immune checkpoint inhibitors
c) Non-specific immunotherapies (e.g. cytokines like interferons and interleukins)
d) Oncolytic viruses
e) Immune cells, e.g. T cells, NK cells, or NKT cells, e.g. CAR T-cells
f) Cancer vaccines.

In one example the immunotherapeutic agent may be an immune checkpoint inhibitor (ICI), cancer vaccine or an immune cell.

In one example the immunotherapeutic agent is an immune checkpoint inhibitor, optionally wherein the immune checkpoint inhibitor is selected from the group consisting of a: PD-1 inhibitor, PD-L1 inhibitor, CTLA4 inhibitor, TIGIT inhibitor, TIM-3 inhibitor, LAG-3 inhibitor, B7-H3 inhibitor, B7-H4 inhibitor.

In one example the immune checkpoint inhibitor is a PD-1 inhibitor. For example, the ATM inhibitor may be selected from the group consisting of KU-55933, KU-60019, KU-59403, CP-466722, AZ31/AZ32, AZD0156, and AZD1390; and the immune checkpoint inhibitor may be a PD-1 inhibitor. In a specific example, the ATM inhibitor may be AZD1390 and the immune checkpoint inhibitor is a PD-1 inhibitor.

In one example the immune checkpoint inhibitor is a PD-L1 inhibitor. For example, the ATM inhibitor may be selected from the group consisting of KU-55933, KU-60019, KU-59403, CP-466722, AZ31/AZ32, AZD0156, and AZD1390; and the immune checkpoint inhibitor may be a PD-L1 inhibitor. In a specific example, the ATM inhibitor may be AZD1390 and the immune checkpoint inhibitor is a PD-L1 inhibitor.

In one example the immunotherapeutic agent is a cancer vaccine, for example a DNA vaccine. An exemplary DNA vaccine against the E7 protein of HPV (human papilloma virus) is described in Allen A, et al., “Linear doggybone DNA vaccine induces similar immunological responses to conventional plasmid DNA independently of immune recognition by TLR9 in a preclinical model.” Cancer Immunol Immunother 2018; 67:627-38. Therapeutic cancer vaccines directed at other viral antigens (eg EBV, HBV, HBC antigens), shared tumour antigens (eg Her2/Neu, hTERT, Muc1, cancer testis antigens) and neoantigens are also applicable.

In one example the immunotherapeutic agent is an immune cell. Several appropriate immune cells are known in the art. For example, the immunotherapeutic agent may be an immune cell selected from a T cell, NK cell or NKT cell. These immune cells are known to be useful as cell therapies. In other words, the immunotherapeutic agent may be an agent that is used in immune cell therapy. In one example, the immune cell is a T cell, for example a T cell that comprises a chimeric antigen receptor (CAR) or T cell receptor (TCR) that targets a tumour associated antigen (TAA) on a target tumour cell. Accordingly, the agent may be a CAR-T cell therapy.

The compounds (i.e. the ATM inhibitor and/or a CHK2 inhibitor and the immunotherapeutic agent), combinations, formulations and/or compositions described herein are for administration in an effective amount. An “effective amount” (or “therapeutically effective amount”) is an amount that alone, or together with further doses, produces the desired (therapeutic) response. The (therapeutically) effective amount to be used will depend, for example, upon the therapeutic objectives, the route of administration, and the condition of the subject. A suitable dosage of the ATM inhibitor and/or a CHK2 inhibitor and the immunotherapeutic agent for a given subject can be determined by an attending physician, taking into consideration various factors known to modify the action of drugs including severity and type of disease, body weight, sex, diet, time and route of administration, other medications and other relevant clinical factors. Accordingly, in one example, a suitable dose of the ATM inhibitor and/or a CHK2 inhibitor and the immunotherapeutic agent is selected based on the body weight of the subject. The dosages and schedules may be varied according to the particular disease state and the overall condition of the patient. For example, it may be necessary or desirable to reduce the above-mentioned doses of the components of the combination treatment in order to reduce toxicity. Suitable doses may also be determined for subgroups of subjects, e.g. based on their heredity and/or pharmocogenetic profile(s).

Therapeutically effective dosages may be determined by either in vitro or in vivo methods.

The compounds, combinations, formulations and/or compositions described herein are therefore administered to a subject in an effective amount to produce the desired response. Preferably, the combination(s), formulation(s) and/or composition(s) described herein will provide a beneficial or synergistic effect on the treatment of a solid tumour in a subject in need thereof. A combination treatment is defined as affording a “synergistic effect” or a “synergistic treatment” if the effect is therapeutically superior, as measured by, for example, the extent of the response, the response rate, the time to disease progression or the survival period, to that achievable on dosing one or other of the components of the combination treatment at its conventional dose. For example, the effect of the combination treatment is synergistic if the effect is therapeutically superior to the effect achievable with the ATM inhibitor and/or a CHK2 inhibitor alone or the immunotherapeutic agent alone. Further, the effect of the combination is synergistic if a beneficial effect is obtained in a group of subjects that does not respond (or responds poorly) to the ATM inhibitor and/or a CHK2 inhibitor alone or the immunotherapeutic agent alone. In addition, the effect of the combination treatment is defined as affording a synergistic effect if one of the components is dosed at its conventional dose and the other component is dosed at a reduced dose and the therapeutic effect, as measured by, for example, the extent of the response, the response rate, the time to disease progression or the survival period, is equivalent to or better than that achievable on dosing conventional amounts of either one of the components of the combination treatment. In particular, synergy is deemed to be present if the conventional dose of the ATM inhibitor and/or a CHK2 inhibitor may be reduced without detriment to one or more of the extent of the response, the response rate, the time to disease progression and survival data, in particular without detriment to the duration of the response, but with fewer and/or less troublesome side-effects than those that occur when conventional doses of each component are used.

As used herein, a “combination” comprising an ATM inhibitor and/or a CHK2 inhibitor and an immunotherapeutic agent encompasses a dosage form of an ATM inhibitor and/or a CHK2 inhibitor for use in combination with a distinct dosage form of an immunotherapeutic agent, as well as a dosage form comprising both a an ATM inhibitor and/or a CHK2 inhibitor and an immunotherapeutic agent. “Combined use” and “combination” in the context of the invention therefore also includes a product comprising both an ATM inhibitor and/or a CHK2 inhibitor and an immunotherapeutic agent, as discrete separate dosage forms, in separate containers or e.g. in blisters containing both types of drugs in discrete solid dosage units, e.g. in a form in which the dosage units which have to be taken together or which have to be taken within one day are grouped together in a manner which is convenient for the subject. Said product itself or as a part of a kit may contain instructions for the simultaneous, sequential or separate administration of the discrete separate dosage units, to a subject. Accordingly, the product may comprise at least two compounds (e.g. an ATM inhibitor and/or a CHK2 inhibitor and an immunotherapeutic agent) as discrete separate dosage forms, in a form which is suitable for sequential, separate and/or simultaneous administration.

The compounds, combinations, formulations and/or compositions may be provided in a form which is suitable for sequential (consecutive), separate and/or simultaneous (concurrent) administration to the subject, in any order. For example, the ATM inhibitor and/or a CHK2 inhibitor may be provided in a form that is suitable for sequential, separate and/or simultaneous administration to the immunotherapeutic agent. Accordingly, the ATM inhibitor and/or a CHK2 inhibitor may be administered to the subject at the same time or at a different time (before or after) compared to when the immunotherapeutic agent is administered. In cases where ATM inhibitor and/or a CHK2 inhibitor and an immunotherapeutic agent are administered simultaneously, the ATM inhibitor and/or a CHK2 inhibitor and immunotherapeutic agent may be administered as separate compositions (e.g. separate formulations) that are administered at the same time, or may be administered as a combined composition or formulation that includes both the ATM inhibitor and/or the CHK2 inhibitor and an immunotherapeutic agent.

The compounds, combinations, formulations and/or compositions described herein can be administered to the subject by any conventional route, including oral administration (for example in tablet form), injection or by gradual infusion over time. The administration may, for example, be topical, oral, parenteral, intravenous, intraperitoneal, intramuscular, intravascular, intracavity, intranasal, intracerebral, intratracheal, intralesional, intraperitoneal, rectal, subcutaneous, transdermal, epidural, percutaneous, or by infusion.

The compounds, combinations, formulations and/or compositions described herein may therefore be in a form suitable for the above modes of administration. For example, suitable forms for oral administration include a tablet or capsule; suitable forms for nasal administration or administration by inhalation include a powder or solution; suitable forms for parenteral injection (including intravenous, subcutaneous, intramuscular, intravascular or infusion) include a sterile solution, suspension or emulsion; suitable forms for topical administration include a patch, an ointment or cream; and suitable forms for rectal administration include a suppository. Alternatively, the route of administration may be by injection.

The compositions and/or formulations of the present invention are advantageously presented in unit dosage form. Dosage forms (also called unit doses) are pharmaceutical drug products in the form in which they are marketed for use, with a specific mixture of active ingredients and inactive components (excipients), in a particular configuration (such as a capsule shell, for example), and apportioned into a particular dose. Depending on the route of administration, dosage forms include liquid, solid, and semisolid dosage forms. Common dosage forms include pills, tablet, capsule, drinks or syrups.

Where the administration of the separate formulations of the ATM inhibitor and/or a CHK2 inhibitor and the immunotherapeutic agent is sequential or separate, the delay in administering the second formulation should not be such as to lose the beneficial effect of the combination therapy.

Details with regards to the inhibitors, treatment, tumours, and immunotherapeutic agent are described elsewhere herein and equally apply here.

### Uses

The ATM and CHK2 inhibitors described herein can be used for treating solid tumours.

Accordingly, in another aspect, the present invention provides an ATM (ataxia telangiectasia mutated) inhibitor and/or a CHK2 inhibitor for use in treating a solid tumour, wherein the solid tumour is a myofibroblastic cancer-associated fibroblast (myoCAF)-high or -moderate tumour.

In another aspect, the present invention provides An ATM (ataxia telangiectasia mutated) inhibitor and/or a CHK2 inhibitor for use in treating a solid tumour in combination with at least one immunotherapeutic agent, wherein the solid tumour is a myofibroblastic cancer-associated fibroblast (myoCAF)-high or -moderate tumour.

In another aspect, the present invention provides an immunotherapeutic agent for use in treating a solid tumour in combination with an ATM (ataxia telangiectasia mutated) inhibitor and/or a CHK2 inhibitor, wherein the solid tumour is a myofibroblastic cancer-associated fibroblast (myoCAF)-high or -moderate tumour

In another aspect, the present invention provides an ATM (ataxia telangiectasia mutated) inhibitor and/or a CHK2 inhibitor in combination with at least one immunotherapeutic agent for use in treating a solid tumour, wherein the solid tumour is a myofibroblastic cancer-associated fibroblast (myoCAF)-high or -moderate tumour.

The ATM and CHK2 inhibitors described herein can be used in the manufacture of a medicament for the treatment of a myoCAF-high or -moderate tumour.

Details with regards to the inhibitors, treatment, tumours, and immunotherapeutic agent are described elsewhere herein and equally apply here.

### Formulations and Kits

In another aspect, the present invention provides a pharmaceutical formulation comprising an ATM inhibitor and/or a CHK2 inhibitor, together with at least one immunotherapeutic agent, and at least one pharmaceutically acceptable excipient.

In another aspect, the present invention provides a kit comprising two separate formulations to be used together, the formulations being:
a) a first formulation comprising an ATM (ataxia telangiectasia mutated) inhibitor and/or a CHK2 inhibitor and at least one pharmaceutically acceptable excipient; and
b) a second formulation comprising at least one immunotherapeutic agent and at least one pharmaceutically acceptable excipient.

In one example the pharmaceutical formulation and/or the kit are provided for use in treating a solid tumour, wherein the solid tumour is a myofibroblastic cancer-associated fibroblast (myoCAF)-high or -moderate tumour.

Details with regards to the inhibitors, treatment, formulations, tumours, and immunotherapeutic agent are described elsewhere herein and equally apply here.

Aspects of the invention are demonstrated by the following non-limiting examples.

### Examples

Myofibroblastic cancer-associated fibroblasts (myoCAF)-high and -moderate tumours generally contain few T-cells and respond poorly to immune-checkpoint blockade. Although myoCAF are associated with poor outcome in most solid tumours, the molecular mechanisms regulating myoCAF accumulation remain unclear, limiting the potential for therapeutic intervention. Here, the inventors identify Ataxia-Telangiectasia Mutated (ATM) as a central regulator of the myoCAF phenotype. Differentiating myofibroblasts in vitro and myoCAF cultured ex vivo display activated ATM signaling, and targeting ATM genetically or pharmacologically could suppress and reverse differentiation. ATM activation was regulated by the reactive oxygen species-producing enzyme NOX4, both through DNA damage and increased oxidative stress. Targeting fibroblast ATM in vivo suppressed myoCAF-high tumour growth, promoted intratumoural CD8 T-cell infiltration, and potentiated the response to anti-PD-1 blockade and anti-tumour vaccination. This work identifies a novel pathway regulating myoCAF differentiation and provides a rationale for using ATM inhibitors to overcome CAF-mediated immunotherapy resistance.

### Materials and Methods

### Analysis of Human Tumours

Informed consent was obtained from each patient. All tissue collection and storage were carried out by a HTA (Human tissue authority)-licensed tissue bank. Automated immunostaining of full tissue sections was performed in a UKAS-accredited Cellular Pathology Laboratory in University Hospital Southampton. 4 μm FFPE sections were mounted on Superfrost+(Thermofisher scientific) slides and pre-heated at 60° C. for 30 minutes. All subsequent steps were completed using commercially available visualization systems [Envision FLEX (Dako)] and automated platforms [Dako PT Link (Dako); Autostainer Link48 (Dako)] optimized for use within a clinical diagnostic pathology laboratory. Deparaffinization, rehydration and antigen retrieval were performed using Dako PT links as previously shown [14]. The order of the primary antibody incubations was: 1) CD31 (1:5; DAB-Brown; IR61061-2 Dako); 2) SMA (ready-to-use; AEC-RED; IR61161-2 Dako); 3) pATM-ser1981 (1:200low AEC_RED; ab36810 Abcam); 4) CK (1:5; AE1/AE3; AEC_RED; IR05361-2, Dako).

Multiplex images were generated using a stain clearing based method: nuclear counterstain (hematoxylin) and registration markers (CD31 using DAB) remain permanent, followed by sequential staining and clearing of a transient stain (SMA, pATM and CK using AEC), as previously[14].

SMA immunohistochemistry (IHC; Sigma Aldrich; A2547) on FFPE tissues was performed as previously [14]: in brief, tissues were fixed in 4% paraformaldehyde and embedded in paraffin. 4 μm sections were de-paraffinized, rehydrated and antigen retrieved for 20 minutes at 97° C. using a predefined program on the Dako PT links. Antigen retrieval was performed using Envision FLEX High pH. Endogenous peroxidase activity was blocked using 3% hydrogen peroxide. Mouse Ig blocking reagent (M.O.M kit vector labs) was applied for 1 hour. Primary antibody incubation (1:100) in M.O.M diluent (vector labs) was for 20 minutes. For secondary amplification, M.O.M. Biotinylated anti-Mouse IgG reagentwas applied for 10 minutes followed by Vectorstain elite ABC reagent (vector labs). Chromogenic visualization was completed with 2×5 minute washes in DAB and counterstaining with Haematoxylin. For IHC of CD8 (in house; YTS169), CD4 (BD; RM4-5) and F480 (AbD Serotec; CI:A3-1) tissues were frozen in OCT. 8 μm sections were fixed in 100% acetone for 10 minutes. Endogenous peroxidase activity was blocked using neat peroxidase suppressor (Peirce) for 15 minutes. Sections were blocked for 30 minutes with 2.5% goat serum. Primary antibodies were applied for 2 hours −CD8 (1:800), CD4 (1:100), F480 (1:100). Appropriate species HRP polymer (ImmunoPress, Vector Laboratories) was applied to sections for 30 minutes. Vector NovaRED chromagen substrate (Vector Laboratories) was applied for 2-10 minutes then counterstained with haematoxylin (Vector Laboratories) for 30 seconds. Images were captured using an Olympus CKX41 microscope with Cell B imaging software. % area of staining was calculated using Fiji software (java) by running the ‘color deconvolution’ tool analyzing H DAB. The image was appropriately thresholded based on the ‘color 2’ image. The same thresholding was applied to all images from the same experiment. Regions of interest (margin & core) were confirmed by a pathologist, and each point plotted represents the mean of a minimum of 4 independent fields of view (field diameter=2 mm) from margin and core (one section per mouse). The mean was calculated from 2 or 3 mice/experimental group.

Quantification of SMA staining in mouse tumours was carried out using color thresholding and deconvolution in Fiji. At least three independent fields of view (FoV) of at least three tumours were randomly selected by a consultant pathologist (GJT) to generate a mean value per tumour representing the SMA-positive area %, which was compared between treatment groups.

CD8 and CD4 IHC was performed on frozen tissue embedded in OCT using [YTS169 (CD8; in house) and RM4-5 (CD4; BD) (FIG. 6c & FIG. 12e). 8 μm sections were fixed in 100% acetone for 10 minutes and stained as previously shown [14]. CD8 and CD4 IHC on FFPE was performed using 98941S and 25229S respectively (Cell Signaling; FIG. 6a,g,k,o and FIG. 12a-c,g & 13m,o) using the Leica Bond system according to the manufacturer's instructions (Protocol F mouse 4; HIER 20 MINUTES ER1; Temperature-Ambient; 1:100 dilution; 20 min of NGS blocking incubation). CD8 and CD4 T-cells were counted from ten randomly selected fields of view (FoV) from the tumour by a consultant pathologist (GJT).

### Cell Culture

Human NSCLC cell line H441, HNSCC SCC25, murine cancer cell line TC-1 (lung), MC38 (colon) and human fetal foreskin fibroblasts HFFF2 were purchased from European Collection of Cell Cultures (Public Health England) or ATCC; HNSCC 5PT [32] were provided by l. Mackenzie (Queen Mary University of London, UK). HEK-293T cells and IMR90 lung fibroblasts were provided by Jesus Gil (Imperial College, London, UK). SCC25 were grown in Ham's F12:DMEM (1:1 ratio) medium with 10% FBS (Calbiochem) and 292 μg/ml L-Glutamine. 5PT were cultured in keratinocyte growth medium [28]. HEK-293T, H441, MC38, HFFF2, IMR90 and primary fibroblasts were grown in DMEM supplemented with 10% FBS and 292 μg/ml L-Glutamine. Primary fibroblasts were isolated as previously [20, 28] used at early passage (p1-10). Primary fibroblasts are listed in Table 1. C57BL/6 murine normal lung and colon fibroblasts (MLF and MCF respectively) were grown at 3% oxygen [15]. TC-1 were cultured in RPMI supplemented with 10% FBS and 292 μg/ml L-Glutamine. All cell cultures were routinely tested for mycoplasma contamination. All cells were cultured using standard cell culture plates/flasks (Corning). HFFF2 or IMR90 cells were transfected with 25 μM On-Target pool siRNA (Thermo Scientific/Dharmacon) using Oligofectamine reagent (Life Technologies) as described [28]. Stable knockdown was performed using retro/lentiviral mediated transduction of shRNA plasmids, as previously described [28] followed by cell selection using 1 μg/ml puromycin for 5-7 days. NOX4-inducible HEK-293 cells (kindly provided by Vincent Jaquet) were treated with 1 μg/ml doxycycline [33]. Wild type and mutant (C2991L) ATM over-expression was carried out transfecting HFFF2 with Viafect (Promega) according to manufacturer's instructions and selected for 10 days with 4 mg/ml G418. p-Retrosuper-shATM (ATM #1:912) and pCDNA3.1-Flag-mut-ATM (C2991L; pTP1625 clone) were kindly provided by Yosef Shiloh and Tanya Paull [31] respectively. pLKO.1 murine shATM (TRCN0000012643) and pCDNA3.1-Flag-wt-ATM (31985) were purchased from Sigma and Addgene respectively. 2 ng/ml TGF-3 (TGF-β1; R&D Systems; for 3 days) was used to induce myofibroblast differentiation. 10 ng/ml IL-1pR (R&D Systems) for 72 hours was used to induce iCAF differentiation. Unless otherwise stated, 40 μM GKT137831 (kindly provided by Genkyotex), 13.3 μM KU55933 (ATM inhibitor), 2.5 μM or 0.5 μM KU60019 (ATM inhibitor), 1.5 μM CCT241533 (CHK2 inhibitor), 40 μM Mirin (Mre11 inhibitor), 0.5 or2.5 μM VE-821 (ATR inhibitor), 2 or 10 μM NU-7441 (DNAPK inhibitor), (all from Selleckchem), 1 μM TGFp3-Receptor I inhibitor (Calbiochem) and 0.5 μM AZD0156 (ATM inhibitor provided by AstraZeneca) were added to the cells 1 hour prior to TGF-β1 treatment. All the inhibitors were resuspended in DMSO. To trigger DDR activation and/or cell stress HFFF2 were either irradiated with 2-100Gy using 350Kv X-Ray Irradiation System (Faxitron), or treated with 100 ng/ml Neocarzinostatin for 2 hours (NCS; Sigma), with 2-10 mM H₂O₂(Fisher Scientific) or with 5 μg/ml Cisplatin (Sandoz) for 30 minutes.

TABLE 1
Primary fibroblasts isolated ex vivo.

| Donor | Tissue | Name |
|---|---|---|
| healthy donor | skin | PSF1 |
| healthy donor | colon | PCF1 |
| healthy donor | colon | PCF2 |
| healthy donor | oral | POF1 |
| healthy donor | oral | POF2 |
| cancer patient | HNSCC | CAF A |
| cancer patient | HNSCC | CAF B |
| cancer patient | HNSCC | CAF E |
| cancer patient | NSCLC | 814 |
| cancer patient | NSCLC | 678 |
| cancer patient | NSCLC | 627 |
| cancer patient | NSCLC | 690 |
| cancer patient | NSCLC | 675 |
| healthy mouse | lung | MLF |
| healthy mouse | lung | MCF |

### Comet Assay

10×10³ HFFF2 cells were treated with TGF-β1 for 24 hours or x-irradiated (2Gy) 30 minutes before harvesting, resuspended in 0.6% low melting point agarose (UltraPure LMP Agarose from Life Technologies), placed onto pre-coated slides with 1% normal melting point agarose and coverslipped. Slides were placed on ice for 30 minutes to allow gels to solidify, coverslips removed and slides placed in ice-cold lysis buffer (2.5 M NaCl, 0.1 M EDTA, 10 mmol/L Tris-HCl, 1% TritonX-100 at pH 10) in the dark, overnight. Next, (with light protection), slides were washed for 10 minutes with ice-cold ddH₂O and placed in ice-cold alkaline buffer (300 mM NaOH, 1 mM disodium EDTA pH>13) for 20 minutes, and electrophoresed for 20 minutes at 30 v/300 mA protected from light. Following electrophoresis, slides were incubated with neutralization buffer (0.4M Tris-HCl, pH 7.5) for 20 minutes, washed twice for 10 minutes with ddH₂O and placed at 37° C. overnight to dry. Slides were rehydrated with ddH₂O for 30 minutes at room temperature, stained with propidium iodide (PI, 2.5 μg/mL) for a further 20 minutes, washed with fresh ddH₂O for 20 minutes and placed at 37° C. to dry before the comet scoring using an Olympus BH-2-RFL-T2 fluorescence microscope (Komet Analysis software version 5.5; Andor Technology). The percentage of DNA in the tail of the comet resulting from total DNA breaks (single and double strand breaks) was calculated for each cell. Six independent experiments were carried out with 50 cells/comets analyzed per replicate.

### Invasion Assays

CAF, HFFF2 or shCTR/shATM HFFF2 populations were treated with 13.3 μM KU55933+/−TGF-β1 for 7 days (treated day 1, day 4). Cells were washed twice with PBS and serum-free DMEM added for 72 hours. Fibroblast-conditioned medium (CM) was collected, centrifuged, filtered (0.2 μm), normalized according to cell number and used as a chemoattractant in the lower chamber of Transwell invasion assays as previously [4, 20]. Two-way Anova was used for statistical analysis.

### Fluorescence Microscopy

5×10³ HFFF2 were plated on permanox chamber slides (Thermo Scientific) and treated as described in the figure legends. Immunostaining was performed as previously [28]. DAPI was used as nuclear counterstain and coverslips were mounted using fluorescent mounting solution (Dako). Images were taken using an Olympus IX81 fluorescence microscope and Xcellence program. DNA Damage foci were quantified from at least 8 non-overlapping FoV with ~300 cells in total per condition. Kruskall-Wallis statistical test was used to indicate the presence of a different distribution per time points in each group of foci. For collagen 1A1 deposition, 5×10³ HFFF2 or MLF were seeded into chamber slides and treated with TGF-β1±inhibitors for a week (treated day 1 & day 4). Decellularisation was performed using 0.25M NH₄OH in 50 mM Tris at 37° C. for 30 minutes and slides fixed with ice-cold 100% methanol at −20° C. followed by a blocking step. The remainderwas performed as previously described [28]. Antibodies used immunofluorescence are listed in Table 2. SMA and Collagen 1A1 quantification was performed using Fiji. High resolution imaging of mouse tumours was carried out using an Axioscan.Z1 scanner (Zeiss). Immunocytochemistry for NOX4 was performed using antibodies from Novus Biologicals and Abcam with similar results (see Table 2 for antibody details).

TABLE 2
Antibody details.

| | | | Dilution | | |
|---|---|---|---|---|---|
| | | | for | Conditions | Concentration |
| | | Cat | western | for | for |
| Antibody | Company | number | blotting | immunofluorescence | FACS |
| SMA | Sigma | A2547 | 1:1000 | 1:750 1 hour | |
| | | | | at RT | |
| HSC70 | Santa Cruz | Sc-7298 | 1:2000 | | |
| pH2Ax-ser139 | Merck/Millipore | 05-636 | 1:500 | 1:100 over- | |
| | | | | night at 4° C. | |
| pSMAD2/3- | Cell Signaling | 3108 | 1:500 | | |
| ser465/7 | | | | | |
| SMAD2 | Cell Signaling | 3122 | 1:500 | | |
| pKAP-1- | Cambridge | A300- | 1:500 | | |
| ser824 | Biosciences | 767A-M | | | |
| KAP-1 | BD Biosciences | 610334 | 1:500 | | |
| FN-ED-A | Merck/Millipore | MAB1940 | 1:1000 | | |
| Phospho- | Cell Signaling | 2851 | 1:500 | | |
| (Ser/Thr) | | | | | |
| ATM/ATR | | | | | |
| Substrate | | | | | |
| NOX4 | Novus Biologicals | NB110- | 1:500 | 1:100 over- | |
| | | 58851 | | night at 4° C. | |
| NOX4 | Abcam | ab1333 | 1:1000 | 1:100 over- | |
| | | 303 | | night at 4° C. | |
| pATM-ser1981 | Rockland | 200- | 1:500 | 1:500 over- | |
| | | 301-400 | | night at 4° C. | |
| ATM | Abcam | Ab59541 | 1:500 | | |
| ATM | Abcam | Ab78 | 1:500 | | |
| pCHK2-thr68 | Cell Signaling | 2197 | 1:500 | | |
| CHK2 | Santa Cruz | sc-8812 | 1:500 | | |
| PATR-ser428 | Cell Signaling | 2853 | 1:500 | | |
| ATR | Santa Cruz | Sc-1887 | 1:250 | | |
| pDNAPKcs- | Abcam | ab124918 | 1:500 | | |
| ser2056 | | | | | |
| DNAPKcs | Cell Signaling | 4602 | 1:500 | | |
| pp53-ser15 | Cell Signaling | 9286 | 1:500 | | |
| p53 | Santa Cruz | Sc-126 | 1:500 | | |
| HSP90 | Santa Cruz | sc-69703 | 1:1000 | | |
| 53BP1 | Cell Signaling | 4937 | 1:1000 | | |
| denatured | Novus Biologicals | NBP1- | 1:500 | | |
| collagen1a1 | | 30054 | | | |
| Collagen1a1 | Abcam | 34710 | | 1:400 1.5 | |
| | | | | hours at RT | |
| denatured | Abcam | ab138492 | 1:500 | | |
| collagen1a1 | | | | | |
| Mre11 | Novus Biologicals | NB100- | 1:500 | | |
| | | 142 | | | |
| Fibronectin | Sigma | F3648 | 1:500 | 1:800 over- | |
| | | | | night at 4° C. | |
| CD8a APC- | eBioscience | 47- | | | 0.2 mg/ml |
| e780 (53-6.7) | | 0081-82 | | | |
| Ki-67 | Biolegend | 652424 | | | 0.2 mg/ml |
| PerCPcy5.5 | | | | | |
| (16a8) | | | | | |
| PD-1 | Biolegend | 109120 | | | 0.2 mg/ml |
| PerCPcy5.5 | | | | | |
| (RMP1-30) | | | | | |
| CD3 FITC | Biolegend | 100204 | | | 0.2 mg/ml |
| (17A2) | | | | | |
| CD107a FITC | Biolegend | 121606 | | | 0.5 mg/ml |
| (1D4B) | | | | | |
| CD107b FITC | Biolegend | 108504 | | | 0.5 mg/ml |
| (M3/84) | | | | | |

### Viability, Proliferation and Toxicity Assays

To assess cell viability and proliferation HFFF2s were plated in 96-well plates (5×10³ cells/well) and treated the following day with inhibitors+/−TGF-β1. After 3 days, [H₃]-thymidine was added to the medium for 16 hours and radioactivity measured using TopCount NXT (Packard Bioscience). MTT assay was carried out using CellTiter 96 AQueous Non-Radioactive Cell Proliferation Assay (Promega) according to manufacturer's instructions. The absorbance was read at 490 nm using Victor plate reader (Perkin Elmer). To monitor cell death, HFFF2s were plated in 6-well plates (200×10³ cells/well) and treated the following day with inhibitors+/−TGF-β1. After 3 days, cells were detached, resuspended in 300 μl of FACs buffer containing 1.7ng/μl Propidium Iodide (ThermoFisher) and analyzed using a Bd FACSCanto Flow Cytometer (BD Biosciences). Cells were less than 90% confluent at the end of each experiment. Cells were treated in the final 30 minutes with 10 mM H₂O₂(for PI and thymidine assay) or 5 μg/ml cisplatin (for MTT assay) as positive controls.

### Senescence and ROS Assay

HFFF2s were plated in 24-well plates (1×10³ or 5×10³ cells/well) and treated with TGF-β1 the following day (and then every 3 days). Senescence was quantified at day 5 and 12 from TGF-β1 treatment determining the percentage of SA-β-Gal-positive cells (Sigma) of the total number of cells counterstained with Dapi (Sigma) as previously described [20]. At least 200 cells/well in at least three independent experiments were counted per sample using a CKX41 Olympus fluorescent microscope. Cells were less than 50% confluent at the end of the experiment to avoid SA-β-Gal-false positive cells due to cell contact. ROS assay was performed as previously[28].

### Western Blotting

Cells were lysed in ice-cold RIPA buffer [1% NP₄0, 150 mM NaCl, 25 mM Tris-HCl pH 7.5, 0.1% SDS, 1% Na-Deoxycolate, 1 mM EGTA, 1 mM EDTA, 1 mM Na-orthovanadate, 10 mM Na-fluoride, 2.5 mM Na-pyrophosphate, 1 mM p glycerophosphate+freshly added 1 mM PMSF, 20 mM N-Ethylmaleimide (NEM), 10 ng/ml Microcystin-LR, 0.12 μM Okadaic acid and phosphatase and protease inhibitor cocktails; all from Sigma]. Protein extracts were then centrifuged and supernatants quantified using Dc-Bio-Rad protein assay kit (Bio-Rad laboratories) as previously [28]. For nuclear-cytoplasmic protein extraction, the inventors used NE-PER Reagents according to manufacturer's instructions (Thermofisher). Equal amounts of protein were electrophoresed either in reducing (+Dithiothreitol, DTT) or in non-reducing conditions in 3-8% or 4-12% SDS-PAGE gels and electro blotted as previously [28]. To detect the ATM dimer, cells were detached, washed once with PBS and then kept moving at room temperature for 10 minutes with PBS+100 mM NEM before nuclear extraction. HSC70 was used as a loading control. Bound antibodies were detected using a chemiluminescence system (Pierce), visualized at the Fluor-S Multi-imager (Bio-Rad) and quantified using Fiji and were plotted as ratios between a given protein and HSC70 unless otherwise stated. Antibodies are described in Table 2.

### Quantitative Real-Time PCR

For Quantitative Real-time PCR (Q-RT-PCR), RNA was extracted and retro-transcribed as described previously [14] and Q-RT-PCR was performed as previously [28]. The mRNA expression levels were analyzed using the AACt relative quantitation method. GAPDH or HPRT were used as human or mouse housekeeping genes respectively. Gene specific primers were designed using http://www.ncbi.nlm.nih.gov/tools/primer-blast/. Primer sequences are displayed in Table 3.

### Collagen Gel Contraction Assays

5×10⁵ fibroblasts were added to 1 ml of ice-cold 3 mg/ml type 1 collagen (Millipore) gels containing 10% FBS DMEM as previously [21]. Gels were incubated at 37° C. for 1 hour to polymerize. After adding 1 ml of 10% FBS DMEM, gels were detached using a spatula and photographed after 24-hours incubation. Gel area was measured using Fiji.

### In Vivo Mouse Experiments

All mouse experiments were performed according to national and international guidelines and were approved by the authors' institutional review board and the UK Home Office. Xenograft model: 6.7×10⁵ PT cells±2×10⁶ HFFF2 cells were resuspended in 100 μl PBS and injected s.c. in the flank of partially immunocompromised, male RAG1−/−C57BL/6 mice (3-5 months old). Isograft models: 0.5×10⁵ TC-1 cells±3×10⁵ TGF-β1-treated murine lung fibroblasts (myoMLFs) or 1×10⁵ MC38 cells±5×10⁵ TGF-β1-treated murine colon fibroblasts (myoMCFs) were resuspended in 100 μl PBS and injected s.c. in the flank of C57/BL6 female mice (6-8 weeks old). 5-8 animals for both models were used per group. Tumour size was measured over time using an electronic caliper and calculated using the formula 4π/3×r3 [radius (r) calculated from the average diameter, measured as the tumour width and length]. AZD0156 (ATMin; provided by AstraZeneca) was tested only on TC-1 and MC38 models (5PT cells were generated using prolonged cisplatin treatment and potentially have an altered DDR pathway[32]). Vaccination with DNA vaccine encoding tetanus Fragment C domain 1 (Dom) fused to the immunodominant CD8 epitope of HPV E7 RAHYNIVTF (RAH, E749-57) p.Dom-RAH [34] was administered once via intramuscular injection (i.m) when tumours were palpable (day 8-13; 20 μg of DNA in 100 μl PBS). P.Dom without the epitope served as a control. 300 μg of αPD-1 antibody (Bioxcell; RMP1-14) or IgG2a isotype control were given via intraperitoneal (i.p) injection when tumours were palpable every other day, totaling 3 doses starting from day 9-14 from the tumour challenge. When tumours were palpable (at day 8-21 for TC-1 model and at day 8-9 for the MC38 model), mice were treated by oral gavage daily with either vehicle or 20 mg/Kg AZD0156 (ATMin) until the end of the experiment (as for manufacturer's instructions).

For TC-1 survival analysis, mice were injected with RAH/CTR vaccines on day 14; AZD0156 was administered daily from days 15-47 and then twice a week until the end of the experiment (day 82). For MC38 survival analysis, αPD-1 was administered on days 14, 16 and 18; AZD0156 was given daily from day 9-28 and then twice weekly until the end of the experiment (day 47).

To test the effect of re-treatment following relapse (FIG. 13e-o), mice were injected with MC38+myoMCF and treated when tumours were palpable with αPD-1 (at day 12, 14 & 16)+/−AZD0156 (20 mg/kg; day 6-16; daily; first treatment). At relapse (tumour size≥500 mm³), a second treatment with αPD-1 (2 doses; spaced at 48 hours when tumour size≥700/800 mm³)+/−AZD0156 (tumour size≥500 mm³; 20 mg/kg; daily treatment for 10 days) was administered. Tumours were collected for IHC after the first treatment, at relapse and when they reached the tumour size limit (1750 mm³). Mice whose tumours were used for IHC, whose size was ≥700/800 mm³, or had regressed completely after the first treatment (2/17 after ATMin+αPD-1 and 0/10 after αPD-1) were not included in the second treatment.

The area under the curve (AUC) for each individual tumour within a treatment group for multiple experiments was analyzed by ordinary Two-way Anova using as two independent variables ‘the experiments’ (factor 1) and ‘the treatments’ (factor 2), and as dependent variable ‘the AUC’ of each mouse; then, the inventors looked at just factor 2. Following euthanasia tumours were excised and either fixed in 10% formalin and embedded in paraffin or directly embedded in OCT matrix (Thermo Scientific) for IHC processing or disaggregated for FACS staining as previously (EF506 viability dye was used for live/dead cell staining; see Table 2 for the antibodies) [14].

### Bioinformatic Analysis

Laser capture microdissected (LCMD) stroma from GEO datasets [GSE45001 (liver), GSE35602 (colon), GSE19632 (esophagus) and GSE40595 (ovary)] were downloaded from https://www.ncbi.nlm.nih.gov/gds/, confirmed to be median normalized, pre-ranked based on fold change (adj.p-vals0.05) between tumour vs normal stroma and GSEA was run against DDR/ATM, TGFβ and myoCAF (myCAF) genesets (https://www.gsea-msigdb.org/gsea/msigdb/) [12, 20, 28]. Normalized Enrichment Score (NES)≥0 indicates positive correlation and B&H adj-p-val (FDRQ) 50.05 was considered significant. The DDR_TGFB_signature geneset was created using the Leading-Edge tool in GSEA from a previously published dataset (TGF_UP_MELLONE_AGING) [20]. Briefly, TGF-β DEGs were ranked based on fold change and GSEA performed on genesets containing reference to DNA Damage Repair in their description and also on H_G2M_checkpoint and H_DNA_repair genesets. Genesets with significant enrichment (FDRQ≤0.25, according to GSEA guidelines) were selected manually excluding the ones not strictly correlated with DNA damage and repair (the full list is provided in Table 4). The list of the leading-edge genes in these genesets was made non-redundant (Table 5) and used to analyze the DDR enrichment in the GEO datasets mentioned above.

TABLE 4
Significant DDR genesets (from Leading-Edge analysis in GSEA)
used to make the DDR_TGFB_signature geneset.

| | | | | | | | RANK | |
|---|---|---|---|---|---|---|---|---|
| | | | | NOM | FDR | FWER | AT | LEADING |
| NAME | SIZE | ES | NES | p-val | q-val | p-val | MAX | EDGE |
| ZHOU_CELL_{—} | 83 | 0.47 | 1.58 | 0.01 | 0.11 | 0.06 | 4406 | tags = 43%, |
| CYCLE_GENES_{—} | | | | | | | | list = 25%, |
| IN_IR_{—} | | | | | | | | signal = 58% |
| RESPONSE_{—} | | | | | | | | |
| 6HR | | | | | | | | |
| HALLMARK_{—} | 200 | 0.35 | 1.34 | 0.02 | 0.13 | 0.36 | 6406 | tags = 52%, |
| G2M_CHECKPOINT | | | | | | | | list = 37%, |
| | | | | | | | | signal = 81% |
| DNA_REPAIR | 122 | 0.37 | 1.34 | 0.03 | 0.12 | 0.37 | 5822 | tags = 44%, |
| | | | | | | | | list = 33%, |
| | | | | | | | | signal = 66% |
| REACTOME_{—} | 104 | 0.39 | 1.37 | 0.03 | 0.16 | 0.30 | 5736 | tags = 43%, |
| DNA_REPAIR | | | | | | | | list = 33%, |
| | | | | | | | | signal = 64% |
| KEGG_{—} | 28 | 0.51 | 1.40 | 0.06 | 0.18 | 0.26 | 4813 | tags = 50%, |
| HOMOLOGOUS_{—} | | | | | | | | list = 28%, |
| RECOMBINATION | | | | | | | | signal = 69% |
| KEGG_BASE_{—} | 33 | 0.50 | 1.42 | 0.07 | 0.23 | 0.23 | 5878 | tags = 61%, |
| EXCISION_{—} | | | | | | | | list = 34%, |
| REPAIR | | | | | | | | signal = 91% |
| KAUFFMANN_{—} | 224 | 0.32 | 1.23 | 0.07 | 0.19 | 0.63 | 5822 | tags = 39%, |
| DNA_REPAIR_{—} | | | | | | | | list = 33%, |
| GENES | | | | | | | | signal = 58% |
| GO_MISMATCH_{—} | 30 | 0.49 | 1.36 | 0.09 | 0.14 | 0.33 | 5736 | tags = 50%, |
| REPAIR | | | | | | | | list = 33%, |
| | | | | | | | | signal = 74% |
| MURAKAMI_{—} | 16 | 0.54 | 1.33 | 0.13 | 0.11 | 0.39 | 5012 | tags = 69%, |
| UV_RESPONSE_{—} | | | | | | | | list = 29%, |
| 24HR | | | | | | | | signal = 96% |
| RESPONSE_{—} | 157 | 0.31 | 1.15 | 0.16 | 0.27 | 0.80 | 5822 | tags = 41%, |
| TO_DNA_{—} | | | | | | | | list = 33%, |
| DAMAGE_STIMULUS | | | | | | | | signal = 61% |
| HALLMARK_{—} | 148 | 0.31 | 1.14 | 0.18 | 0.24 | 0.82 | 5423 | tags = 38%, |
| DNA_REPAIR | | | | | | | | list = 31%, |
| | | | | | | | | signal = 55% |
| KAUFFMANN_{—} | 138 | 0.31 | 1.12 | 0.21 | 0.25 | 0.84 | 5096 | tags = 34%, |
| DNA_REPLICATION_{—} | | | | | | | | list = 29%, |
| GENES | | | | | | | | signal = 48% |
| KEGG_MISMATCH_{—} | 23 | 0.44 | 1.15 | 0.26 | 0.25 | 0.80 | 5012 | tags = 35%, |
| REPAIR | | | | | | | | list = 29%, |
| | | | | | | | | signal = 49% |

### Statistical Analysis

All experiments were performed at least twice and data are expressed as the mean+/−standard error (SE) unless otherwise stated (SD=standard deviation). Data are presented as group with number of biological replicates shown in the figure and the number of technical replicates described in the figure legends (n_{br} or nₜᵣ respectively) and summarized by mean+95% confidence intervals (CIs) in all figures. The appropriate use of parametric vs nonparametric tests was determined using the D'Agostino and Pearson omnibus normality test, and where sample sizes were too small for this test, a normal distribution was assumed. Grubbs test was used to identify outliers. Where data did not follow a normal distribution pattern, Kruskall-Wallis, Mantel-Cox (log-rank) or Spearman tests were used. For normal distributions, Two-way Anova, or homo/paired/heteroscedastic Student's t-test were used as described in the figure legends. Statistical tests were carried out using GraphPad Prism v. 6-8, were two-sided; a p value 50.05 was considered significant (ns=non-significant, *p<0.05, **p<0.01, ***p<0.001, ****p<0.0001).

### Results

### ATM Activation During Myofibroblast Differentiation

To investigate the DDR pathway during myofibroblast differentiation, the inventors treated fibroblasts with TGF-β1, and examined activation of DDR kinases over time alongside myofibroblast markers [SMA, Fibronectin EDA (FnEDA)]. The inventors found activation of ATM (but not ATR or DNAPKcs) during differentiation (FIG. 1a and FIG. 7a-e); while some ATM targets (H2AX, CHK2) were activated following TGF-β1 treatment, others (p53, KAP1) were not (FIG. 1b and FIG. 7e), indicating that only a subset of ATM targets typically phosphorylated during genotoxic stress are activated during myofibroblast differentiation. The inventors also found that TGF-β1 treatment resulted in increased (albeit low) levels of pATM/pH2AX-positive nuclear foci and DNA breaks (FIG. 1c-e). This low level of DNA damage was associated with non-significant trends for reduced DNA replication and increased cell death; there were no changes in cell viability (monitored 3 days post TGF-β1 treatment) or senescence (5 days; FIG. 1f&g). However, as previously shown [35], continuous TGF-β1 administration resulted in increased senescence levels over time (% range SA-β-Gal-positive cells at 12 days=15.8-23.3; FIG. 1g) suggesting overall that accumulation of DNA damage induced by TGF-β1 may ultimately promote cell senescence in a proportion of myofibroblasts.

Next, the inventors examined OAF cultured ex vivo and human tumours in vivo for evidence of ATM activation. OAF from Non-Small Cell Lung Cancer (NSCLC) and Head & Neck (HNSCC) showed ATM activation in vitro, which correlated with SMA expression (Spearman's correlation=0.96, pval=0.0028; FIG. 1h). Gene set enrichment analyzes of micro-dissected tumour stroma datasets showed that myoCAF stroma of esophageal, ovarian, colorectal and liver cancers are enriched for genes associated with ATM/DDR pathway (FIG. 1ii & Table 5). Multiplex immunochemistry (MxIHC) confirmed that the majority of SMA-positive CAF in NSCLC and HNSCC express pATM (FIG. 1j-n & FIG. 7f-i) indicating, overall, that myoCAF in vitro and in vivo display activated ATM signaling.

TABLE 5
GSEA between different tumour stromal datasets
from GEO and DDR & myoCAF genesets.

| | | |
|---|---|---|
| Genesets | NES | FDR q-val |

Colon 35602

| | | |
|---|---|---|
| TRANSFORMING GROWTH FACTOR-BETA | 4.17498 | 0.0005 |
| HUMAN FIBROBLASTS GDS4574 LIGAND: 18 | | |
| TGF-UP_MELLONE_AGING | 3.9959 | 0.0005 |
| HALLMARK_G2M_CHECKPOINT | 3.81127 | 0.0005 |
| ECM_MYCAF | 3.72347 | 0.0005 |
| TGF-BETA HUMAN PROSTATE FIBROBLASTS | 3.30487 | 0.0005 |
| GSE68164 LIGAND: 240 | | |
| VERRECCHIA_EARLY_RESPONSE_TO_TGFB1 | 3.15817 | 0.0005 |
| WOUND_MYCAF | 2.89227 | 0.0005 |
| VERRECCHIA_DELAYED_RESPONSE_TO_TGFB1 | 2.81901 | 0.0005 |
| TGF_MYCAF | 2.7308 | 0.0005 |
| GO_DNA_REPAIR | 2.62909 | 0.0005 |
| DDR_TGFB_SIGNATURE | 2.52914 | 0.0005 |
| KYNG_DNA_DAMAGE_BY_GAMMA_AND_UV_RADIATION | 2.35308 | 6.7E−05 |
| HALLMARK_DNA_REPAIR | 1.98758 | 0.00274 |

Ovary 40595

| | | |
|---|---|---|
| ECM_MYCAF | 2.80631 | 0.0005 |
| TRANSFORMING GROWTH FACTOR-BETA | 2.79609 | 0.0005 |
| HUMAN FIBROBLASTS GDS4574 LIGAND: 18 | | |
| TGF-BETA HUMAN PROSTATE FIBROBLASTS | 2.34258 | 0.0005 |
| GSE68164 LIGAND: 240 | | |
| VERRECCHIA_EARLY_RESPONSE_TO_TGFB1 | 2.3158 | 0.0005 |
| WOUND_MYCAF | 2.3148 | 0.0005 |
| TGF_MYCAF | 2.07801 | 0.0004 |
| HALLMARK_DNA_REPAIR | 2.02853 | 0.0004 |
| HALLMARK_G2M_CHECKPOINT | 1.95644 | 0.0012 |
| VERRECCHIA_DELAYED_RESPONSE_TO_TGFB1 | 1.89789 | 0.0062 |
| TGF-UP_MELLONE_AGING | 1.8937 | 0.0062 |
| KYNG_DNA_DAMAGE_BY_GAMMA_AND_UV_RADIATION | 1.89217 | 0.0062 |
| DDR_TGFB_SIGNATURE | 1.76917 | 0.0284 |
| GO_DNA_REPAIR | 1.65849 | 0.0736 |

Liver 45001

| | | |
|---|---|---|
| KYNG_DNA_DAMAGE_BY_GAMMA_AND_UV_RADIATION | −1.2065 | 0.23982 |
| HALLMARK_G2M_CHECKPOINT | 3.25261 | 0.0005 |
| GO_DNA_REPAIR | 3.19269 | 0.0005 |
| TGF-UP | 2.95325 | 0.0005 |
| TRANSFORMING GROWTH FACTOR-BETA | 2.73581 | 0.0005 |
| HUMAN FIBROBLASTS GDS4574 LIGAND: 18 | | |
| DDR_TGFB_SIGNATURE | 2.72688 | 0.0005 |
| TGF-BETA HUMAN PROSTATE FIBROBLASTS | 2.59044 | 0.0005 |
| GSE68164 LIGAND: 240 | | |
| TGF_MYCAF | 2.48643 | 6.5E−05 |
| ECM_MYCAF | 2.35024 | 0.00027 |
| HALLMARK_DNA_REPAIR | 1.81926 | 0.01591 |
| VERRECCHIA_EARLY_RESPONSE_TO_TGFB1 | 1.34427 | 0.13472 |
| WOUND_MYCAF | 0.95546 | 0.49793 |

EAC 19632

| | | |
|---|---|---|
| DDR_TGFB_SIGNATURE | −0.72 | 0.83397 |
| TRANSFORMING GROWTH FACTOR-BETA | 2.90727 | 0.0005 |
| HUMAN FIBROBLASTS GDS4574 LIGAND: 18 | | |
| TGF-UP | 2.76858 | 0.0005 |
| TGF-BETA HUMAN PROSTATE FIBROBLASTS | 2.64113 | 0.0005 |
| GSE68164 LIGAND: 240 | | |
| ECM_MYCAF | 2.35995 | 0.0005 |
| HALLMARK_G2M_CHECKPOINT | 2.22257 | 0.0002 |
| VERRECCHIA_DELAYED_RESPONSE_TO_TGFB1 | 2.06766 | 0.00158 |
| TGF_MYCAF | 2.06279 | 0.00136 |
| WOUND_MYCAF | 1.90891 | 0.00531 |
| GO_DNA_REPAIR | 1.76884 | 0.01507 |
| KYNG_DNA_DAMAGE_BY_GAMMA_AND_UV_RADIATION | 1.42561 | 0.10515 |
| VERRECCHIA_EARLY_RESPONSE_TO_TGFB1 | 1.23312 | 0.21988 |
| HALLMARK_DNA_REPAIR | 1.17423 | 0.2543 |

### Targeting ATM Inhibits Myofibroblast Differentiation

To determine if ATM plays a functional role in myofibroblast differentiation, the inventors co-treated fibroblasts with TGF-β1 and an ATM inhibitor (KU55933). ATM inhibition suppressed differentiation, inhibiting expression of SMA (FIG. 2a,c & FIG. 8a-h), contraction of collagen gels, SMA-positive stress fiber formation and collagen1 deposition (FIG. 2d,e & FIG. 8i). The inventors validated these findings using a second ATM inhibitor (KU60019) and ATM si/shRNA knockdown, which resulted in similar reduction of SMA (ACTA2) (FIG. 2b,f,g & FIG. 8j-l) and expression of ECM genes (COL1A1 & CTGF genes; FIG. 2g). Overexpression of a dominant negative Mut-ATM (C2991L) [31, 36] also suppressed expression of SMA and ECM genes (FIG. 8m-o). Targeting ATR or DNAPK did not reduce SMA expression (FIG. 2s) consistent with their lack of activation during myofibroblast differentiation (FIG. 7e).

Recent single cell transcriptomic studies have identified an inflammatory CAF (iCAF) subpopulation in several cancer types, and described CAF plasticity, whereby cells in culture can be skewed between myoCAF and iCAF phenotypes by modulating TGF-β1 and IL1 signaling respectively [12, 37]. Therefore, the inventors compared the effect of ATM inhibition on expression of myoCAF and iCAF genes. KU55933 suppressed TGF-β1 induction of myoCAF genes (COL11A1, ASPN, ELN) and significantly upregulated IL-1p induction of most iCAF genes (CCL2, LIF, IL6, CXCL1) (FIG. 8p-s). This hints at a dual role for ATM in determining fibroblast phenotype: as promoter of myofibroblast differentiation and as inhibitor of inflammatory CAF phenotype.

Next, the inventors investigated whether CHK2, a downstream target of ATM, is also involved in myofibroblast differentiation. For this, the inventors used a CHK2-specific inhibitor CCT241533 [38] and found, similar to ATM inhibition, that it suppresses TGF-β1-dependent SMA and FnEDA expression, SMA-positive stress fiber formation and collagen deposition (FIG. 2l,m and FIG. 8t,u). siRNA targeting CHK2 produced a similar effect, decreasing expression of SMA (ACTA2), COL1A1, CTGF (FIG. 2n,o). Overall, these data indicate that ATM and its downstream signaling are central to the development of the myofibroblast phenotype.

ATM Maintains the Established myoCAF Phenotype

Continued ATM activation in differentiated myoCAF in vitro and in vivo (FIG. 1h-n) suggest that it may play a role in maintaining the established myoCAF phenotype. Therefore, the inventors examined the effect of ATM targeting on HNSCC and NSCLC CAF cultured ex vivo. The inventors found that inhibiting ATM using KU55933 or shRNA-knockdown, reverted CAF to a more quiescent phenotype, downregulating expression of myofibroblast markers (SMA, collagen I, fibronectin; FIG. 3a-d), suppressing contractility (FIG. 3e) and inhibiting myoCAF-dependent cancer cell invasion (FIG. 3f,g). Similarly, ATM inhibition or knockdown suppressed the invasion-promoting effects of myofibroblasts generated through TGF-β1 treatment (FIG. 9a-c). Together these data highlight that ATM can be targeted to both inhibit myoCAF differentiation and also ‘normalize’ established myoCAF.

### TGF-β1 Activates ATM Via NOX4

Next, the inventors investigated the molecular mechanisms underlying TGF-β1-dependent activation of ATM. First, the inventors inhibited TGF-β-receptor I phosphorylation of SMAD2/3 and confirmed that this abolished ATM activation in TGF-β1-treated fibroblasts (FIG. 4a). NOX4 is a SMAD downstream target [39], which the inventors have shown previously regulates myoCAF differentiation [28]; it has been reported that the ROS generated by this enzyme can cause DNA damage suggesting a possible nuclear localization for NOX4 [27]. The inventors found that ATM phosphorylation and NOX4 expression increased with similar kinetics during the myofibroblast differentiation process (FIG. 1a & FIG. 7a-c) and both localized to the cell nucleus (FIG. 4b & FIG. 10a-c) but did not specifically co-localize (FIG. 4c). Targeting NOX4 using siRNA or a NOX4 inhibitor (GKT137831) [28] suppressed ROS production (FIG. 10d), and inhibited ATM phosphorylation, as well as its downstream kinase activity (FIG. 4d,e). DNA damage monitored by pH2AX protein expression was also decreased (FIG. 10e,f). Since ATM is classically activated through DNA damage recognition by the MRN complex [29, 30], the inventors examined whether Mre11 was directly involved in TGF-β1-dependent activation of ATM. Targeting Mre11 using siRNA or using a specific Mre11 inhibitor (Mirin) inhibited TGF-β1-driven ATM activation/activity similar to NOX4 inhibition (FIG. 4d,e). Consistent with this, Mre11 inhibition suppressed myofibroblast differentiation (FIG. 4f-j and FIG. 10g,h).

Given that ATM can also be activated as a covalent dimer by oxidative stress [31], the inventors examined whether NOX4-driven ROS promotes ATM dimerization. First, the inventors confirmed ATM dimer (ATM-D) formation by oxidation (H₂O₂; FIG. 10i), before examining the effect of NOX4 inhibition. The inventors found that NOX4 targeting resulted in a marked reduction in basal and TGF-β1-induced levels of the ATM dimer (FIG. 4k & FIG. 10l). Targeting Mre11 did not affect ATM dimerization (FIG. 10j,k) consistent with the MRN complex being involved in TGF-β1 activation of the monomer. Targeting NOX4 using GKT also inhibited ATM activation and dimerization in HNSCC and NSCLC CAF (FIG. 10m-q). To confirm the role of NOX4 in ATM activation, the inventors overexpressed NOX4 in HEK-293 cells [33]. This induced ATM activation, dimerization and activity (the latter monitored by CHK2 phosphorylation) without the requirement for TGF-β1 stimulation (FIG. 4l,m), suggesting that NOX4 activation and oxidation of ATM are not cell type-specific. These data, summarized in the schematic in FIG. 4n, show that activation of ATM during myofibroblast differentiation is modulated by NOX4 both through DNA damage/MRN complex and also direct oxidation of ATM.

### ATM Targeting in Myofibroblasts Reduces Tumour Growth

Next, the inventors tested whether ATM targeting affects tumour growth in vivo using different murine tumour models [14, 28]. First, the inventors co-injected immunocompromised mice with 5PT cancer cells, a cell line that promotes myoCAF differentiation [28], with either control HFFF2 fibroblasts or HFFF2 with ATM shRNA knock-down (FIG. 5a). Stromal ATM targeting attenuated myoCAF accumulation and suppressed tumour growth (FIG. 5b-e). To investigate the role of fibroblast ATM targeting in immunocompetent mouse models, and to overcome the issue of low CAF content in commonly used syngeneic murine tumours, the inventors used a syngeneic isograft lung cancer model as previously [14], co-injecting TC-1 cancer cells with TGF-β1-treated murine lung fibroblasts (MLF), which recapitulates the myoCAF-high stroma of human tumours (FIG. 11a-c). The inventors found that myofibroblast ATM shRNA knock-down similarly suppressed intratumoural myoCAF accumulation and reduced tumour size in this model (FIG. 5f-j).

The inventors then evaluated the effect of AZD0156, a clinically-tested ATM-specific inhibitor (NCT02588105). The inventors confirmed that AZD0156 suppressed myofibroblast differentiation in vitro (FIG. 11d-g) and also reversed myoCAF differentiation; notably, this latter effect was maintained when AZD0156 treatment was discontinued (FIG. 11h). A broad analysis of myoCAF, iCAF and other immune genes showed that most myoCAF genes were significantly down-regulated by ATM inhibition whereas the effect on iCAF genes was gene-specific and generally non-significant (FIG. 11i,j). Next, the inventors tested the effect of the inhibitor on mice injected with TC-1 cells+/−TGF-β1-treated MLF (myoMLF). In mice bearing myoCAF-high TC-1 tumours, AZD0156 suppressed myofibroblast accumulation, slowed tumour growth and improved overall survival (FIG. 5k-o and FIG. 11k,l). However, AZD0156 had minimal effect on the growth of control (myoCAF-low) tumours, showing that the effect of AZD0156 is specific to myoCAF in this model (FIG. 5k,l). Similar results were obtained using an isogenic colorectal cancer mouse model, co-injecting MC38 cancer cells with TGF-β-treated mouse colorectal fibroblasts (myoMCF; FIG. 5p-s). Together these data show that ATM can be targeted to suppress intratumoural myoCAF accumulation resulting in reduced tumour growth.

ATM myoCAF Targeting Increases Intratumoural CD8 T-Cell Infiltration and Potentiates Immunotherapy

The inventors have shown previously that myoCAF confer immunotherapy resistance by excluding CD8 T-cells from tumours [14]. Therefore, the inventors tested whether ATM inhibition could reverse this effect. Analysis of myoCAF-high TC-1 and MC38 tumours by IHC showed that ATM inhibition (fibroblast shRNA knockdown and AZD0156) resulted in a significant relocation of CD8 T-cells from the tumour periphery to the tumour core (FIG. 6a-d,g,h and FIG. 12a,b). CD4 T-cell infiltration was unaffected (FIG. 12c-h). Flow cytometry analysis showed that ATM inhibition did not affect CD8 T-cell phenotype/function in myoCAF-high tumours (FIG. 6e,f).

Next, the inventors investigated whether the effect of ATM inhibition on intratumoural CD8 T-cells could potentiate response to immunotherapy. First, the inventors tested a vaccine model using HPV E6/E7-expressing TC-1 cells, combining AZD0156 with a DNA vaccine directed against E7 (RAH) [34]; the inventors have shown previously that myoCAF-high TC-1 tumours are resistant to the vaccine response [14]. Mice bearing CAF-rich TC-1 tumours were treated with AZD0156 or vaccine monotherapy, vaccine/drug combination or control. While AZD0156 monotherapy was effective in reducing intratumoural myoCAF accumulation (FIG. 12i,j), the drug/vaccine combination significantly increased CD8 T-cell infiltration and reduced tumour volume compared to the single treatments (FIG. 6i-l and FIG. 13a). AZD0156 was also tested in combination with αPD-1 in mice bearing myoCAF-high MC38 tumours, a model where, similarly, myoCAF confer resistance to anti-PD-1 therapy [14]. The inventors found again that the combination of αPD-1 with AZD0156 produced the most significant CD8 T-cell influx and reduction on tumour growth (FIG. 6m-p and FIG. 12k,l & 13b). Survival experiments performed over a longer time period showed that combining ATM inhibition with immunotherapy increased the overall survival compared to single treatments alone in both tumour models (FIG. 6q,r and FIG. 13c,d). When tumours relapsed upon αPD1+/−AZD0156, a second treatment with the combination was administered resulting in significantly fewer myoCAF and increased CD8 T-cells (FIG. 13e-g). Notably, despite the high levels of CD8 T-cells in these tumours, mice showed only a non-significant trend for increased survival (FIG. 13h-o). Overall, these data show that ATM inhibition is effective at promoting infiltration of CD8 T-cells into myoCAF-high tumours and can be used to potentiate the early response to immunotherapy.

### DISCUSSION

MyoCAF are associated with poor prognosis in many cancers, and have been shown to promote tumour immune evasion. However, effective targeting of this cell population has not yet been accomplished [16, 19], in part confounded by the fact that CAF remain a poorly-defined, heterogeneous cell population [7, 17]. Recent single cell transcriptomic analyzes have identified novel CAF phenotypes including iCAF and antigen presenting CAF (apCAF), although it is not yet clear how widely these CAF subgroups are found in different cancers or how they function[40]. Most research has focused on myoCAF. These are present to a greater or lesser extent in most types of solid tumours, usually abutting tumour cells, and depositing a desmoplastic stroma rich in collagens, fibronectin and proteoglycans that has been shown to ‘trap’ T-cells and limit T-cell access to the tumour core [14, 41, 42]. Notably, expression of TGF-β-associated myoCAF ECM genes is one of the strongest predictors of immunotherapy failure, highlighting the ability of myoCAF to create an immunosuppressive tumour microenvironment [8-12]. Here, the inventors show that activation of ATM plays a central role in promoting and maintaining the myoCAF phenotype and that ATM can be effectively targeted to ‘normalize’ myoCAF, overcome myoCAF-mediated immune evasion and potentiate response to immunotherapy.

ATM, along with ATR and DNA-PKcs, is principally known for its role as an apical kinase in the DDR pathway. However, ATM signaling has been shown to also regulate other cell functions including glucose metabolism[43], cell homeostasis[44], and multiple differentiation processes [45]. The inventors found that TGF-β1-induced myofibroblast differentiation results in activation of ATM and CHK2 in presence of significantly increased (albeit low) DNA breaks. The localized pattern of pH2AX and pATM staining in the nuclei of cells treated with TGF-β1 confirmed the presence of DNA damage in the form of DSBs [30], consistent with TGFβ/SMAD signaling promoting ATM activation and downstream signaling as a result of direct DNA damage [25-27, 46]. In keeping with this, the inventors found that inhibiting MRN complex/DNA damage activation of ATM by targeting Mre11 inhibits TGF-β1 induction of the kinase.

In breast CAF, ATM has also been shown to be activated by oxidative stress in the absence of DNA damage, suggesting potential activation of the ATM dimer [47]. Here, the inventors found that ATM activation results from both oxidation and DNA damage, and that both are dependent on ROS generated by NOX4. The inventors established that NOX4 (but not Mre11) activates ATM as a dimer, indicating a ROS-driven oxidation of the kinase independent of monomer activation by the MRN complex. Consistent with NOX4 being reported to promote DDR activation in several cell types [25, 27], here the inventors demonstrate that NOX4 is the source of ROS promoting ATM dimer formation during the myofibroblast/myoCAF differentiation process. This likely explains the lack of KAP1 activation typically observed during oxidative stress activation of ATM [31] and proposes a model whereby there exists a parallel activation of ATM by DNA damage and oxidative stress, both induced by NOX4.

The inventors found that ATM was activated in myoCAF isolated ex vivo from NSCLC and HNSCC, also detected by multiplexed immunochemistry in the SMA-positive stroma of the same tumours. GSEA also showed enrichment of ATM/DDR-related genes in the myofibroblastic stroma of ovarian, esophageal, liver and colorectal tumours, consistent with proteomic analysis of esophageal and breast CAF performed elsewhere [48, 49]. In support of its role in myoCAF differentiation, ATM activation has additionally been reported to contribute to various fibrotic conditions, including systemic sclerosis [50], and renal [51] and hepatocellular fibrosis [52], all myofibroblast-dependent processes, suggesting, overall, that the association of ATM signaling and myofibroblast/myoCAF phenotypes holds true across different tissues and disease pathogenesis. Continued ATM activation in myoCAF isolated ex vivo suggested that it also plays a role in maintaining the myoCAF phenotype. The inventors found that ATM inhibition normalized myoCAF, downregulating SMA and other myoCAF markers while promoting an iCAF phenotype, consistent with recent studies that have highlighted plasticity in CAF populations, showing that CAF subgroups are not fixed in state of terminal differentiation[37, 53].

The inventors also investigated whether DDR upstream or downstream components of the ATM pathway can regulate myofibroblastic CAF phenotype and found, that Mre11 and CHK2 are also involved in myofibroblast differentiation. This contrasts with previous work that identified CHK2 as repressor of breast myofibroblastic CAF phenotype [54], although is consistent with the increased DDR gene expression and ATM activation observed in breast CAF by several other groups [47, 49]

ATM signaling involvement in myoCAF differentiation is in keeping with previous data showing that DNA damaging agents, including irradiation, can induce a contractile, SMA-positive myofibroblastic-like phenotype through an ATM-dependent mechanism [20]. Additionally, the inventors have shown that continuous TGF-β1 treatment of fibroblasts over time promotes senescence [35], which is commonly triggered by DNA damage. It appears therefore that myofibroblast activation and senescence may be two linked stress responses, perhaps with TGF-β1 induction of ATM signaling and the myofibroblast phenotype, followed by later senescence forming part of the same differentiation program.

The recognition that myoCAF provide a major resistance mechanism to immunotherapy has renewed interest in CAF targeting as an immunotherapy adjunct, and different therapeutic approaches have been suggested. TGF-β signaling is the major pathway regulating myoCAF differentiation, and different groups have shown that co-administration of anti-TGF-β with anti-PD-1/PD-L1 antibodies significantly improves response in preclinical tumour models [8, 13]. TGFβ blockage has been shown to reduce the formation of myoCAF and promote the formation of an ‘interferon-licensed’ CAF subpopulation with increased immunomodulatory properties, resulting in greater anti-PD-1 efficacy [53]. Inhibiting TGF3, however, can be problematic; this pleiotropic cytokine has multiple roles in normal physiology as well as tumour suppressive effects, and its targeting has led to on-target cardiac toxicities in preclinical studies, as well as the development of cutaneous tumours in human trials [18, 55]. Inhibiting the mechanisms by which CAF exclude T-cells from tumours is an alternative approach to overcome immunotherapy resistance and perhaps the most attractive strategy to do that is to ‘normalize’ myoCAF, particularly since studies have suggested that certain CAF/fibroblast phenotypes may be tumour-suppressive [56]. Previously, the inventors identified the ROS-producing enzyme NOX4 as key regulator of the myoCAF phenotype [14, 28]. Inhibition of NOX4 using the small molecule inhibitor GKT137831/Setanaxib suppresses myoCAF differentiation and overcomes myoCAF-mediated immunotherapy resistance [14]. Here, the inventors found that ATM is activated by NOX4 and plays a similar role in regulating and maintaining myoCAF differentiation.

Drugs inhibiting ATM and other components of the DDR are generally effective in tumours with specific pre-existing DNA repair defects or are used in combination with platinum compounds or ionizing radiation where they are associated with significant toxicity [57]. As monotherapy, ATM inhibitors are well tolerated, with dose escalation phase 1 clinical studies with the ATM inhibitors AZD0156 or AZD1390 not yet reporting to be associated with adverse effects. However, ATM inhibitor monotherapy is generally therapeutically ineffective in conventional pre-clinical tumour models [58, 59](where the stromal component is typically absent [14]). Nonetheless, recent studies have shown that ATM inhibitors can promote response to checkpoint immunotherapy through cGAS/STING signaling in tumour cells, resulting in an enhanced interferon I response and increased immunogenicity[58]. Notably, The inventors found that ATM inhibition did not affect control (CAF-low) tumours comprising TC-1 and MC38 cells alone, but its effect was limited to myoCAF-high tumours, where it reversed myoCAF differentiation and potentiated immunotherapy response. Whether this latter effect is modulated through the downregulation of ECM proteins or altered expression of inflammatory cytokines remains to be determined [41, 58], but this data shows that the use of ATM inhibitors can be expanded for stromal targeting.

In summary, the inventors identify ATM activation as a novel, targetable pathway regulating myoCAF differentiation. Most types of solid tumours have a myoCAF-high subgroup; given the major role of myoCAF in suppressing response to anti-PD-1/PD-L1 checkpoint inhibitors [8, 13], targeting this pathway as part of combination immunotherapy could have significant therapeutic benefit.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

TABLE 3
SEQUENCES

| | Primer | | SEQ ID |
|---|---|---|---|
| Gene | direction | DNA sequence (5′ to 3′) | NO: |
| human COL1A1 | F | ACGAAGACATCCCACCAATCACCT | 1 |
| | R | AGATCACGTCATCGCACAACACCT | 2 |
| human ATM | F | GCAACTGTACCAAGAAGAGTCCAA | 3 |
| | R | GCAGGAAAAAGCCAGATGCA | 4 |
| human CTGF | F | CCCTCGCGGCTTACCGACTG | 5 |
| | R | GGCGCTCCACTCTGTGGTCT | 6 |
| human NOX4 | F | GCTGACGTTGCATGTTTCAG | 7 |
| | R | CGGGAGGGTGGGTATCTAA | 8 |
| human MRE11A | F | GTCCGTGAGGCTATGACCAG | 9 |
| | R | GTTGGTTGCTGCTGAGATGC | 10 |
| human GAPDH | F | AGCAATGCCTCCTGCACCACCAAC | 11 |
| | R | CCGGAGGGGCCATCCACAGTCT | 12 |
| human ACTA2 | F | GACAATGGCTCTGGGCTCTGTAA | 13 |
| | R | ATGCCATGTTCTATCGGGTACTT | 14 |
| human ATR | F | GCTATGCTACTAGTGGGCCG | 15 |
| | R | GCACGCGGTCACATCCTTAT | 16 |
| human CHEK2 | F | TGACAACTACTGGTTTGGGAG | 17 |
| | R | CCCACTTCCCTGAAAATCCGA | 18 |
| human FN1 | F | TGTGGTTGCCTTGCACGA | 19 |
| | R | GCTTGTGGGTGTGACCTGAGT | 20 |
| human PRKDC | F | ACCTGAAGCGCCGCTATAAT | 21 |
| | R | GGAAGGACCATCTGAATCCCC | 22 |
| human F10 | F | GCTCGGGGAAAGTCTGTTCA | 23 |
| | R | GCAGGTCTCTTCCATGCACT | 24 |
| human ELN | F | GGCCATTCCTGGTGGAGTTC | 25 |
| | R | CGAGACCAGCCCCTGGATAA | 26 |
| human COL3A1 | F | AATCAGGTAGACCCGGACGA | 27 |
| | R | TTCGTCCATCGAAGCCTCTG | 28 |
| human ASPN | F | ACAAGAGAGCCAAGAAGCCA | 29 |
| | R | TGGGACTGAGGTCAAACCTAAA | 30 |
| human COL11A1 | F | AGGAACCTCAGATAGATGAGAAAAA | 31 |
| | R | TGATTTAGTAGCCACTGTCCTCA | 32 |
| human CCL2 | F | AGCTCGCACTCTCGCCTCCAG | 33 |
| | R | GGCATTGATTGCATCTGGCTGAGC | 34 |
| human IL6 | F | CCAGGAGCCCAGCTATGAAC | 35 |
| | R | CCCAGGGAGAAGGCAACTG | 36 |
| human CXCL1 | F | GAAAGCTTGCCTCAATCCTG | 37 |
| | R | CACCAGTGAGCTTCCTCCTC | 38 |
| human LIF | F | TCTTGGCGGCAGGAGTTG | 39 |
| | R | GTTGTTGTGACATGGGTGGC | 40 |
| mouse ATM | F | CGATATGCCAGTCTTTTCAGGG | 41 |
| | R | CTCAAGGCTGCCCTTACTCA | 42 |
| mouse HPRT | F | GTTGGGCTTACCTCACTGCT | 43 |
| | R | TCATCGCTAATCACGACGCT | 44 |
| mouse COL1A1 | F | GTGTTCCCTACTCAGCCGTC | 45 |
| | R | ACTCGAACGGGAATCCATCG | 46 |
| mouse ACTA2 | F | CCTCATGCCATCATGCGTCT | 47 |
| | R | AATCTCACGCTCGGCAGTAG | 48 |
| mouse COL3A1 | F | TCCTGGTGGTCCTGGTACTG | 49 |
| | R | AGGAGAACCACTGTTGCCTG | 50 |
| mouse COL11A1 | F | CAGCGTGGTGAGACTGGATT | 51 |
| | R | GGATGGCCACGTTCACCTAT | 52 |
| mouse COL1A2 | F | GCTGGTGTAATGGGTCCTCC | 53 |
| | R | CGACCGGCATCTCCATTAGG | 54 |
| mouse CTGF | F | AGAACTGTGTACGGAGCGTG | 55 |
| | R | GTGCACCATCTTTGGCAGTG | 56 |
| mouse FN1 | F | GAAGACAGATGAGCTTCCCCA | 57 |
| | R | GGTTGGTGATGAAGGGGGTC | 58 |
| mouse POSTN | F | TCATTTAGAGCAGCCGCCAT | 59 |
| | R | GCAAAGAGCGTGAAGTGACC | 60 |
| mouse VCAN | F | CTGGCTGTGGATGGTGTTGT | 61 |
| | R | CAGAGTGTACCTGCTGGTGG | 62 |
| mouse CTHRC1 | F | TGAAGCAAAAAGCGCTGATCC | 63 |
| | R | ACTCCTGCTGGTCCTTGTAG | 64 |
| mouse ELN | F | AGTCTATTATCCAGGGGCTGGT | 65 |
| | R | CTGCTCCAAACGTTCCCAGA | 66 |
| mouse ASPN | F | GGGATAGAACCAGGGGCATT | 67 |
| | R | GTTGGTGGTAGGCCTTTTGGA | 68 |
| mouse CXCL10 | F | CCACGTGTTGAGATCATTGCC | 69 |
| | R | TCACTCCAGTTAAGGAGCCC | 70 |
| mouse IRF3 | F | AACCCTGCACCCCAAGAAAA | 71 |
| | R | ACCTCGAACTCCCATTGTTCC | 72 |
| mouse ISG15 | F | CTAGAGCTAGAGCCTGCAG | 73 |
| | R | AGTTAGTCACGGACACCAG | 74 |
| mouse IFIT1 | F | CAAGGCAGGTTTCTGAGGAG | 75 |
| | R | GACCTGGTCACCATCAGCAT | 76 |
| mouse CCL2 | F | CACTCACCTGCTGCTACTCA | 77 |
| | R | GAGCTTGGTGACAAAAACTACAGC | 78 |
| mouse CCL5 | F | ATCTTGCAGTCGTGTTTGTCA | 79 |
| | R | ACCCACTTCTTCTCTGGGTTG | 80 |
| mouse IL6 | F | TGATTGTATGAACAACGATGATGC | 81 |
| | R | GGACTCTGGCTTTGTCTTTCTTGT | 82 |
| mouse LIF | F | AGCGCCAATGCTCTCTTCAT | 83 |
| | R | CTTCTCTGTCCCGTTGCCAT | 84 |
| mouse CXCL1 | F | ACTCAAGAATGGTCGCGAGG | 85 |
| | R | ACTTGGGGACACCTTTTAGCA | 86 |
| mouse CXCL12 | F | TGCATCAGTGACGGTAAACCA | 87 |
| | R | GCGATGTGGCTCTCGAAGAA | 88 |
| mouse CXCL2 | F | GCCCAGACAGAAGTCATAGCC | 89 |
| | R | GGCTTCAGGGTCAAGGCAA | 90 |

Q-RT-PCR primers. F: forward and R: reverse.

### REFERENCES

1. Fearon, D. T., Immune-Suppressing Cellular Elements of the Tumor Microenvironment. Annual Review of Cancer Biology, Vol 1, 2017. 1: p. 241-255.
2. Kalluri, R., The biology and function of fibroblasts in cancer. Nat Rev Cancer, 2016. 16(9): p. 582-98.
3. Hinz, B., et al., Recent developments in myofibroblast biology: paradigms for connective tissue remodeling. Am J Pathol, 2012. 180(4): p. 1340-55.
4. Marsh, D., et al., Stromal features are predictive of disease mortality in oral cancer patients. J Pathol, 2011. 223(4): p. 470-81.
5. Underwood, T. J., et al., Cancer-associated fibroblasts predict poor outcome and promote periostin-dependent invasion in oesophageal adenocarcinoma. J Pathol, 2015. 235(3): p. 466-77.
6. Yamashita, M., et al., Role of stromal myofibroblasts in invasive breast cancer: stromal expression of alpha-smooth muscle actin correlates with worse clinical outcome. Breast Cancer, 2012. 19(2): p. 170-6.
7. Hanahan, D. and L. M. Coussens, Accessories to the crime: functions of cells recruited to the tumor microenvironment. Cancer Cell, 2012. 21(3): p. 309-22.
8. Mariathasan, S., et al., TGFbeta attenuates tumour response to PD-L1 blockade by contributing to exclusion of T cells. Nature, 2018. 554(7693): p. 544-548.
9. Hugo, W., et al., Genomic and Transcriptomic Features of Response to Anti-PD-1 Therapy in Metastatic Melanoma. Cell, 2017. 168(3): p. 542.
10. Chakravarthy, A., et al., TGF-beta-associated extracellular matrix genes link cancer-associated fibroblasts to immune evasion and immunotherapy failure. Nat Commun, 2018. 9(1): p. 4692.
11. Dominguez, C. X., et al., Single-Cell RNA Sequencing Reveals Stromal Evolution into LRRC15(+) Myofibroblasts as a Determinant of Patient Response to Cancer Immunotherapy. Cancer Discov, 2020. 10(2): p. 232-253.
12. Kieffer, Y., et al., Single-Cell Analysis Reveals Fibroblast Clusters Linked to Immunotherapy Resistance in Cancer. Cancer Discov, 2020. 10(9): p. 1330-1351.
13. Tauriello, D. V. F., et al., TGFbeta drives immune evasion in genetically reconstituted colon cancer metastasis. Nature, 2018. 554(7693): p. 538-543.
14. Ford, K., et al., NOX4 Inhibition Potentiates Immunotherapy by Overcoming Cancer-Associated Fibroblast-Mediated CD8 T-cell Exclusion from Tumors. Cancer Res, 2020.
15. Togo, S., et al., Carcinoma-associated fibroblasts are a promising therapeutic target. Cancers (Basel), 2013. 5(1): p. 149-69.
16. Narra, K., et al., Phase II trial of single agent Val-boroPro (Talabostat) inhibiting Fibroblast Activation Protein in patients with metastatic colorectal cancer. Cancer Biol Ther, 2007. 6(11): p. 1691-9.
17. Sugimoto, H., et al., Identification of fibroblast heterogeneity in the tumor microenvironment. Cancer Biol Ther, 2006. 5(12): p. 1640-6.
18. Morris, J. C., et al., Phase I study of GC1008 (fresolimumab): a human anti-transforming growth factor-beta (TGFbeta) monoclonal antibody in patients with advanced malignant melanoma or renal cell carcinoma. PLoS One, 2014. 9(3): p. e90353.
19. Catenacci, D. V., et al., Randomized Phase Ib/II Study of Gemcitabine Plus Placebo or Vismodegib, a Hedgehog Pathway Inhibitor, in Patients With Metastatic Pancreatic Cancer. J Clin Oncol, 2015. 33(36): p. 4284-92.
20. Mellone, M., et al., Induction of fibroblast senescence generates a non-fibrogenic myofibroblast phenotype that differentially impacts on cancer prognosis. Aging (Albany NY), 2016. 9(1): p. 114-132.
21. Jackson, S. P. and J. Bartek, The DNA-damage response in human biology and disease. Nature, 2009. 461(7267): p. 1071-8.
22. Blackford, A. N. and S. P. Jackson, ATM, ATR, and DNA-PK: The Trinity at the Heart of the DNA Damage Response. Mol Cell, 2017. 66(6): p. 801-817.
23. Barcellos-Hoff, M. H. and F. A. Cucinotta, New tricks for an old fox: impact of TGFbeta on the DNA damage response and genomic stability. Sci Signal, 2014. 7(341): p. re5.
24. Kirshner, J., et al., Inhibition of transforming growth factor-beta1 signaling attenuates ataxia telangiectasia mutated activity in response to genotoxic stress. Cancer Res, 2006. 66(22): p. 10861-9.
25. Hubackova, S., et al., IFNgamma induces oxidative stress, DNA damage and tumor cell senescence via TGFbeta/SMAD signaling-dependent induction of Nox4 and suppression of ANT2. Oncogene, 2016. 35(10): p. 1236-49.
26. Dickey, J. S., et al., Intercellular communication of cellular stress monitored bygamma-H2AX induction. Carcinogenesis, 2009. 30(10): p. 1686-95.
27. Razdan, N., T. Vasilopoulos, and U. Herbig, Telomere dysfunction promotes transdifferentiation of human fibroblasts into myofibroblasts. Aging Cell, 2018. 17(6): p. e12838.
28. Hanley, C. J., et al., Targeting the Myofibroblastic Cancer-Associated Fibroblast Phenotype Through Inhibition of NOX4. J Natl Cancer Inst, 2018. 110(1).
29. Paull, T. T., Mechanisms of ATM Activation. Annu Rev Biochem, 2015. 84: p. 711-38.
30. Bakkenist, C. J. and M. B. Kastan, DNA damage activates ATM through intermolecular autophosphorylation and dimer dissociation. Nature, 2003. 421(6922): p. 499-506.
31. Guo, Z., et al., ATM activation by oxidative stress. Science, 2010. 330(6003): p. 517-21.
32. Bauer, J. A., et al., Reversal of cisplatin resistance with a BH3 mimetic, (−)-gossypol, in head and neck cancer cells: role of wild-type p53 and Bcl-xL. Mol Cancer Ther, 2005. 4(7): p. 1096-104.
33. Serrander, L., et al., NOX4 activity is determined by mRNA levels and reveals a unique pattern of ROS generation. Biochem J, 2007. 406(1): p. 105-14.
34. Allen, A., et al., Linear doggybone DNA vaccine induces similar immunological responses to conventional plasmid DNA independently of immune recognition by TLR9 in a pre-clinical model. Cancer Immunol Immunother, 2018. 67(4): p. 627-638.
35. Hassona, Y., et al., Progression of genotype-specific oral cancer leads to senescence of cancer-associated fibroblasts and is mediated by oxidative stress and TGF-beta. Carcinogenesis, 2013. 34(6): p. 1286-95.
36. Guo, Z., R. Deshpande, and T. T. Paull, ATM activation in the presence of oxidative stress. Cell Cycle, 2010. 9(24): p. 4805-11.
37. Biffi, G., et al., IL1-Induced JAK/STAT Signaling Is Antagonized by TGFbeta to Shape CAF Heterogeneity in Pancreatic Ductal Adenocarcinoma. Cancer Discov, 2019. 9(2): p. 282-301.
38. Anderson, V. E., et al., CCT241533 is a potent and selective inhibitor of CHK2 that potentiates the cytotoxicity of PARP inhibitors. Cancer Res, 2011. 71(2): p. 463-72.
39. Bai, G., et al., A far-upstream AP-1/Smad binding box regulates human NOX4 promoter activation by transforming growth factor-beta. Gene, 2014. 540(1): p. 62-7.
40. Elyada, E., et al., Cross-Species Single-Cell Analysis of Pancreatic Ductal Adenocarcinoma Reveals Antigen-Presenting Cancer-Associated Fibroblasts. Cancer Discov, 2019. 9(8): p. 1102-1123.
41. Salmon, H., et al., Matrix architecture defines the preferential localization and migration of T cells into the stroma of human lung tumors. J Clin Invest, 2012. 122(3): p. 899-910.
42. Evanko, S. P., et al., Hyaluronan and versican in the control of human T-lymphocyte adhesion and migration. Matrix Biol, 2012. 31(2): p. 90-100.
43. Armata, H. L., et al., Requirement of the ATM/p53 tumor suppressor pathway for glucose homeostasis. Mol Cell Biol, 2010. 30(24): p. 5787-94.
44. Lee, J. H., et al., ATM directs DNA damage responses and proteostasis via genetically separable pathways. Sci Signal, 2018. 11(512).
45. Sherman, M. H., C. H. Bassing, and M. A. Teitell, Regulation of cell differentiation by the DNA damage response. Trends Cell Biol, 2011. 21(5): p. 312-9.
46. Legrand, A. J., et al., Persistent DNA strand breaks induce a CAF-like phenotype in normal fibroblasts. Oncotarget, 2018. 9(17): p. 13666-13681.
47. Tang, S., et al., Oxidized ATM promotes abnormal proliferation of breast CAFs through maintaining intracellular redox homeostasis and activating the PI3K-AKT, MEK-ERK, and Wnt-beta-catenin signaling pathways. Cell Cycle, 2015. 14(12): p. 1908-24.
48. Manousopoulou, A., et al., Quantitative proteomic profiling of primary cancer-associated fibroblasts in oesophageal adenocarcinoma. Br J Cancer, 2018. 118(9): p. 1200-1207.
49. Peng, Q., et al., Biological characteristics and genetic heterogeneity between carcinoma-associated fibroblasts and their paired normal fibroblasts in human breast cancer. PLoS One, 2013. 8(4): p. e60321.
50. Svegliati, S., et al., Oxidative DNA damage induces the ATM-mediated transcriptional suppression of the Wnt inhibitor WIF-1 in systemic sclerosis and fibrosis. Sci Signal, 2014. 7(341): p. ra84.
51. Daugherity, E K., et al., The DNA damage checkpoint protein ATM promotes hepatocellular apoptosis and fibrosis in a mouse model of non-alcoholic fatty liver disease. Cell Cycle, 2012. 11(10): p. 1918-28.
52. Overstreet, J. M., et al., Tumor suppressor ataxia telangiectasia mutated functions downstream of TGF-beta1 in orchestrating profibrotic responses. FASEB J, 2015. 29(4): p. 1258-68.
53. Grauel, A. L., et al., TGFbeta-blockade uncovers stromal plasticity in tumors by revealing the existence of a subset of interferon-licensed fibroblasts. Nat Commun, 2020. 11(1): p. 6315.
54. Al-Rakan, M. A., S. F. Hendrayani, and A. Aboussekhra, CHEK2 represses breast stromal fibroblasts and their paracrine tumor-promoting effects through suppressing SDF-1 and IL-6. BMC Cancer, 2016. 16: p. 575.
55. Anderton, M. J., et al., Induction of heart valve lesions by small-molecule ALK5 inhibitors. Toxicol Pathol, 2011. 39(6): p. 916-24.
56. Helms, E., M. K. Onate, and M. H. Sherman, Fibroblast Heterogeneity in the Pancreatic Tumor Microenvironment. Cancer Discov, 2020. 10(5): p. 648-656.
57. Pilie, P. G., et al., State-of-the-art strategies for targeting the DNA damage response in cancer. Nat Rev Clin Oncol, 2019. 16(2): p. 81-104.
58. Hu, M., et al., ATM inhibition enhances cancer immunotherapy by promoting mtDNA leakage and cGAS/STING activation. J Clin Invest, 2021. 131(3).
59. Durant, S. T., et al., The brain-penetrant clinical ATM inhibitor AZD1390 radiosensitizes and improves survival of preclinical brain tumor models. Sci Adv, 2018. 4(6): p. eaat1719.

1. A method of treating a solid tumour in a subject, the method comprising administering a therapeutically effective amount of an ATM (ataxia telangiectasia mutated) inhibitor and/or a CHK2 inhibitor to the subject, wherein the solid tumour is a myofibroblastic cancer-associated fibroblast (myoCAF)-high or -moderate tumour.

5. An ATM (ataxia telangiectasia mutated) inhibitor and/or a CHK2 inhibitor for use in treating a solid tumour, wherein the solid tumour is a myofibroblastic cancer-associated fibroblast (myoCAF)-high or -moderate tumour.

3. A method of treating a solid tumour in a subject, the method comprising administering a therapeutically effective amount of:
a) an ATM (ataxia telangiectasia mutated) inhibitor and/or a CHK2 inhibitor; and
b) at least one immunotherapeutic agent;
to the subject, wherein the inhibitor and the at least one immunotherapeutic agent are administered simultaneously, separately or consecutively, and wherein the solid tumour is a myofibroblastic cancer-associated fibroblast (myoCAF)-high or -moderate tumour.

4. A method of treating a solid tumour in a subject, the method comprising administering a therapeutically effective amount of an ATM (ataxia telangiectasia mutated) inhibitor and/or a CHK2 inhibitor to the subject, wherein the subject is undergoing treatment with, has been treated with, or has been prescribed treatment with at least one immunotherapeutic agent, and wherein the solid tumour is a myofibroblastic cancer-associated fibroblast (myoCAF)-high or -moderate tumour.

5. A method of treating a solid tumour in a subject, the method comprising administering a therapeutically effective amount of at least one immunotherapeutic agent to the subject, wherein the subject is undergoing treatment with, has been treated with, or has been prescribed treatment with an ATM (ataxia telangiectasia mutated) inhibitor and/or a CHK2 inhibitor, and wherein the solid tumour is a myofibroblastic cancer-associated fibroblast (myoCAF)-high or -moderate tumour.

6. An ATM (ataxia telangiectasia mutated) inhibitor and/or a CHK2 inhibitor for use in treating a solid tumour in combination with at least one immunotherapeutic agent, wherein the solid tumour is a myofibroblastic cancer-associated fibroblast (myoCAF)-high or -moderate tumour.

7. An immunotherapeutic agent for use in treating a solid tumour in combination with an ATM (ataxia telangiectasia mutated) inhibitor and/or a CHK2 inhibitor, wherein the solid tumour is a myofibroblastic cancer-associated fibroblast (myoCAF)-high or -moderate tumour.

8. An ATM (ataxia telangiectasia mutated) inhibitor and/or a CHK2 inhibitor in combination with at least one immunotherapeutic agent for use in treating a solid tumour, wherein the solid tumour is a myofibroblastic cancer-associated fibroblast (myoCAF)-high or -moderate tumour.

9. A pharmaceutical formulation comprising an ATM (ataxia telangiectasia mutated) inhibitor and/or a CHK2 inhibitor, together with at least one immunotherapeutic agent, and at least one pharmaceutically acceptable excipient.

10. A kit comprising two separate formulations to be used together, the formulations being:
a) a first formulation comprising an ATM (ataxia telangiectasia mutated) inhibitor and/or a CHK2 inhibitor and at least one pharmaceutically acceptable excipient; and
b) a second formulation comprising at least one immunotherapeutic agent and at least one pharmaceutically acceptable excipient.

11. The pharmaceutical formulation of claim 9, or the kit of claim 10, for use in treating a solid tumour, wherein the solid tumour is a myofibroblastic cancer-associated fibroblast (myoCAF)-high or -moderate tumour.

12. A method for promoting T-cell infiltration and antitumour immunity of a solid tumour in a subject, comprising subjecting the subject to a therapeutically effective amount of an ATM (ataxia telangiectasia mutated) inhibitor and/or a CHK2 inhibitor in combination with at least one immunotherapeutic agent, wherein the solid tumour is a myofibroblastic cancer-associated fibroblast (myoCAF)-high or -moderate tumour.

13. The method, inhibitor, agent, formulation or kit of any one of claims 1 to 12, wherein the subject has been identified as having a myofibroblastic cancer-associated fibroblast (myoCAF)-high or -moderate solid tumour.

14. The method, inhibitor, agent, formulation or kit of any one of claims 1 to 13, wherein the ATM inhibitor and/or the CHK2 inhibitor is used in a neoadjuvant, adjuvant, first line and/or palliative manner.

15. The method, inhibitor, agent, formulation or kit of any one of claims 1 to 14, wherein the solid tumour is:
(a) a myofibroblastic cancer-associated fibroblast (myoCAF)-high tumour when α-smooth muscle actin (SMA) staining of a tumour tissue sample shows a stromal positivity of more than 50%; or (b) a myofibroblastic cancer-associated fibroblast (myoCAF)-moderate tumour when α-smooth muscle actin (SMA) staining of a tumour tissue sample shows a stromal positivity of 5-50%.

16. The method, inhibitor, agent, formulation or kit of any one of claims 1 to 15, wherein the solid tumour is a cancer selected from the group consisting of: non-small cell lung cancer (NSCLC), head and neck squamous cell cancer (HNSCC), esophageal cancer, ovarian cancer, colorectal cancer, liver cancer, breast cancer, ovarian cancer, cervical cancer, endometrial cancer, melanoma, hepatocellular carcinoma, kidney cancer, prostate cancer, gastric cancer, pancreatic cancer, urinary bladder cancer, and brain cancer; optionally wherein the brain cancer is glioblastoma.

17. The method, inhibitor, agent, formulation or kit of any one of claims 1 to 16, wherein the ATM and/or the CHK2 inhibitor prevents or reverses the differentiation of myoCAFs.

18. The method, inhibitor, agent, formulation or kit of any one of claims 1 to 17, wherein the ATM inhibitor directly inhibits ATM and/or wherein the CHK2 inhibitor directly inhibits CHK2.

19. The method, inhibitor, agent, formulation or kit of any one of claims 1 to 18, wherein the ATM inhibitor inhibits ATM by blocking the kinase function of ATM and/or wherein the CHK2 inhibitor inhibits CHK2 by blocking the kinase function of CHK2.

20. The method, inhibitor, agent, formulation or kit of any one of claims 1 to 19, wherein the ATM and/or the CHK2 inhibitor promotes intra-tumoural T-cell infiltration into the solid tumour.

21. The method, inhibitor, agent, formulation or kit of any one of claims 1 to 20, wherein the ATM and/or the CHK2 inhibitor potentiates the solid tumour's response to anti-PD-1 blockade.

22. The method, inhibitor, agent, formulation or kit of any one of claims 1 to 21, wherein the ATM inhibitor is selected from the group consisting of KU-55933, KU-60019, KU-59403, CP-466722, AZ31/AZ32, AZD0156, and AZD1390.

23. The method, inhibitor, agent, formulation or kit of any one of claims 1 to 21, wherein the CHK2 inhibitor is selected from the group consisting of CCT241533, BML-277, PHI-101, VRX0466617, PV1019, AZD7762, and Y2606368 (Prexasertib).

24. The method, inhibitor, agent, formulation or kit of any one of claims 3 to 23, wherein the immunotherapeutic agent is selected from the group consisting of: immune checkpoint inhibitor, a cancer vaccine and/or an immune cell.

25. The method, inhibitor, agent, formulation or kit of any one of claims 3 to 22, wherein the immunotherapeutic agent is an immune checkpoint inhibitor, optionally wherein the immune checkpoint inhibitor is selected from the group consisting of a: PD-1 inhibitor, PD-L1 inhibitor, CTLA4 inhibitor, TIGIT inhibitor, TIM-3 inhibitor, LAG-3 inhibitor, B7-H3 inhibitor, and B7-H4 inhibitor.
